(19) United States
(12) Patent Application Publication
(10) Pub. No.: US 2024/0398785 A1
(43) Pub. Date: Dec. 5, 2024

(54) SMALL MOLECULE DRUGS FOR THE INDUCTION OF TRAINED IMMUNITY

(71) Applicant: THE UNIVERSITY OF CHICAGO, Chicago, IL (US)

(72) Inventors: Aaron ESSER-KAHN, Chicago, IL (US); Hannah KNIGHT, Chicago, IL (US); Jainu AJIT, Chicago, IL (US)

(73) Assignee: THE UNIVERSITY OF CHICAGO, Chicago, IL (US)

(21) Appl. No.: 18/679,407

(22) Filed: May 30, 2024

Related U.S. Application Data
(60) Provisional application No. 63/469,696, filed on May 30, 2023.

(51) Int. Cl. A61K 31/4725; A61K 9/50; A61K 31/045; A61K 31/05; A61K 31/137; A61K 31/352; A61K 31/405; A61K 31/573; A61K 31/58; A61P 37/04

(52) U.S. Cl. CPC A61K 31/4725; A61K 9/5031; A61K 31/045; A61K 31/05; A61K 31/137; A61K 31/352; A61K 31/405; A61K 31/573; A61K 31/58; A61P 37/04

(57) ABSTRACT

The present disclosure provides methods and compositions for inducing trained immunity, preventing infections, improving immune responses, modulating the activity of at least one protein in the BCL family of proteins, increasing the efficacy of a vaccine, and reducing the risk of a secondary tumor in a subject diagnosed with a primary cancer.

### CROSS-REFERENCE TO RELATED APPLICATIONS

This application claims the benefit of priority of U.S. Provisional Patent Application No. 63/469,696 filed May 30, 2023, which is hereby incorporated by reference in its entirety.

### GOVERNMENT INTEREST

This invention was made with government support under 75N93019C00041 awarded by the National Institutes of Health. The government has certain rights in the invention.

### TECHNICAL FIELD

The presently disclosed subject matter relates small molecule compounds capable of inducing trained immunity and to methods of using these compounds, to prevent disease and/or enhance the effectiveness of vaccines in a subject in need thereof, such as an subject with a weakened immune system (e.g., elderly subject, juvenile subjects, and/or subjects taking a medication or having a disease that suppresses the immune system). In some aspects, the methods prevent secondary cancers or diseases resulting from infections. The presently disclosed subject matter also relates to high-throughput screening methods to identify small molecules capable of inducing trained immunity.

### BACKGROUND

Trained immunity is a form of innate immune memory characterized by epigenetic and metabolic reprogramming. This cell rewiring can result in increased cytokine secretion and other effector responses to pathogenic challenges, making it desirable for non-specific protection against diseases for which there are no vaccines. It also can improve immune responses to established immunotherapeutics and vaccines.

Despite the promise of trained immunity for preventing disease, current inducers of trained immunity are generally limited to complex and heterogeneous biologic agents, e.g., pathogen-derived stimuli such as β-glucan and Bacillus Calmette-Gudrin (BCG) vaccines. Recent work has demonstrated that endogenous signaling molecules, including catecholamines, oxidized low density lipoprotein (oxLDL), and aldosterone can induce training in human monocytes.

As a result of the metabolic and epigenetic changes, a trained cell can better react to a secondary trigger. Although trained immunity has beneficial effects, such as better anti-tumor responses in mice, uncontrolled activation and training by many of the listed molecules can be detrimental. Extensive studies have reported the role of trained immunity in allograft rejection, allergy, neurodegenerative disorders, and atherosclerosis. There remains an ongoing need for additional compounds that are non-immunogenic and can induce trained immunity.

### SUMMARY

In some aspects, the presently disclosed subject matter provides a method of inducing trained immunity in a subject, the method comprising administering a small molecule that consists of or comprises a glucocorticoid, A1155463, nerol, hydroquinone, 5-fluoroindole-2-carboxylic acid, myricetin, fenoterol, a pharmaceutically acceptable salt thereof, and any combination thereof. Any one or more of the preceding components may be excluded in certain aspects. In some aspects, administering the small molecule leads to an increase in concentration of at least one cytokine. In some aspects, the increase in concentration of at least one cytokine is an increase of at least 1.6-fold greater than that which would have occurred if the subject had not been administered the small molecule. In some aspects, the at least one cytokine is tumor necrosis factor-alpha (TNF-α) or interleukin-6 (IL-6). In some aspects, the administering comprises administering the small molecule systemically.

In some aspects, administration of a small molecule as disclosed herein does not induce a primary immune response. In some aspects, the small molecule induces a future pro-inflammatory response in the subject. In some aspects, the future pro-inflammatory response occurs when the cell or subject is exposed to an immunological challenge at a time point at least one week after said administration. In some aspects, the immunological challenge is selected from an infectious agent, a secondary metastasis, or a vaccine. In some aspects, the infectious agent consists of or comprises a virus, a bacterium, a protozoan, a prion, a viroid, and a fungus. In some aspects, the immunological challenge is a secondary metastasis and the subject is a subject having previously been treated for a primary cancer.

Some aspects of the disclosure are directed to a method of preventing an infection in a subject, wherein the method comprises administering to the subject a small molecule that consists of or comprises a glucocorticoid, A1155463, nerol, hydroquinone, 5-fluoroindole-2-carboxylic acid, myricetin, fenoterol, a pharmaceutically acceptable salt thereof, and any combination thereof. Any one or more of the preceding components may be excluded in certain aspects. In some aspects, the administering is performed at least one week prior to contact by, or expected risk of contact by, the subject with an infectious agent. In some aspects, the subject is an elderly subject, a juvenile subject, or an immunocompromised subject. In some aspects, the administering is performed at least three weeks prior to contact by, or expected risk of contact by, the subject with an infectious agent. In some aspects, the small molecule does not induce a primary immune response. In some aspects, administration of the small molecule causes an increase in concentration of at least one cytokine. In some aspects, the increase in concentration of at least one cytokine is an increase of at least 1.6-fold greater than that which would have occurred if the subject had not been contacted with the small molecule. In some aspects, the at least one cytokine is tumor necrosis factor-alpha (TNF-α) or interleukin-6 (IL-6).

Some aspects of the present disclosure are directed to a method of reducing a risk of a secondary tumor in a subject diagnosed with a primary cancer. In some aspects, the method comprises treating the subject with a standard regimen of cancer treatment specific for the primary cancer of said subject, and administering to the subject a small molecule that consists of or comprises A1155462, a glucocorticoid, nerol, hydroquinone, 5-fluoroindole-2-carboxylic acid, myricetin, fenoterol a pharmaceutical acceptable salt thereof, and any combination thereof. Any one or more of the preceding components may be excluded in certain aspects. In some aspects, the method reduces the risk of secondary tumor in the subject compared to the risk of secondary tumor in a subject treated for a similar cancer and not administered the small molecule. In some aspects, the standard regimen of treatment comprises chemotherapy, surgical removal of cancer cells, radiation therapy, immunotherapy, or a combination thereof. In some aspects, administration of the small molecule causes an increase in concentration of at least one cytokine. In some aspects, the increase in concentration of at least one cytokine is an increase of at least 1.6-fold greater than that which would have occurred if the subject had not been contacted with the small molecule. In some aspects, the at least one cytokine is tumor necrosis factor-alpha (TNF-α) or interleukin-6 (IL-6). In some aspects, the small molecule inducer of trained immunity does not induce a primary immune response.

Some aspects of the present disclosure are directed to a method of increasing the efficacy of a vaccine administered to a subject. In some aspects, the method comprises administering to the subject a small molecule in an amount sufficient to induce trained immunity in the subject. In some aspects, administration of the small molecule is performed at least one week prior to administration of the vaccine to the subject. In some aspects, the small molecule consists of or comprises a glucocorticoid, A1155463, nerol, hydroquinone, 5-fluoroindole-2-carboxylic acid, myricetin, and fenoterol, a pharmaceutically acceptable salt thereof, and any combination thereof. Any one or more of the preceding components may be excluded in certain aspects. In some aspects, said administering of the small molecule is performed at least three weeks prior to administration of the vaccine. In some aspects, administration of the small molecule causes an increase in concentration of at least one cytokine. In some aspects, the increase in concentration of at least one cytokine is an increase of at least 1.6-fold greater than that which would have occurred if the subject had not been contacted with the small molecule. In some aspects, the at least one cytokine is tumor necrosis factor-alpha (TNF-α) or interleukin-6 (IL-6). In some aspects, the small molecule inducer of trained immunity does not induce a primary immune response.

Some aspects of the present disclosure are directed to a method of modulating the activity of BCL pathway family member protein in a cell of a subject, comprising administering to the subject a modulating effective amount of a small molecule that consists of or comprises a glucocorticoid, A1155463, nerol, hydroquinone, 5-fluoroindole-2-carboxylic acid, myricetin, and fenoterol, a pharmaceutically acceptable salt thereof, and any combination thereof. Any one or more of the preceding components may be excluded in certain aspects. In some aspects, the small molecule modulates the activity of at least one BCL path protein that consists of or comprises Bcl-2, Bcl-XL, Bcl-w, Mcl-1, Bfl1/A-1, Bcl-B, Bax, Bak, Bok, Bid, Bim, Noxa, and/or Puma. In some aspects, the small molecule induces a trained immunity response. In some aspects, the trained immunity response is characterized by an increase in concentration of at least one cytokine. In some aspects, the increase in concentration of at least one cytokine is an increase of at least 1.6-fold greater than that which would have occurred if the subject had not been administered the small molecule. In some aspects, the at least one cytokine is tumor necrosis factor-alpha (TNF-α) or interleukin-6 (IL-6).

In some aspects, a small molecule as disclosed herein is encapsulated in a nanoparticle. In some aspects, a nanoparticle comprises two or more small molecules as disclosed herein. In some aspects, the nanoparticle comprises a biodegradable polymer. In some aspects, the biodegradable polymer consists of or comprises polyesters, polyamides, polyurethanes, polyureas, poly(amide-enamine)s, polyanhydrides, polyvinyl alcohols, polyacrylates, proteins, polysaccharides, natural rubber, and polyolefins. In some aspects, the polyester is poly(lactic-co-glycolic acid).

In some aspects, the glucocorticoid consists of or comprises flunisolide, hydrocortisone, hydrocortisone acetate, halcinonide, budesonide, fluocinonide, fluocinolone acetonide, clobetasol propionate, prednisolone, triamcinolone, triamcinolone diacetate, nitrofurazone, and benseraside hydrochloride. Any one or more of the preceding glucocorticoids may be excluded in certain aspects.

In some aspects, the subject is a mammal. In further aspects, the mammal is a human. In some aspects, the human is an immunocompromised human. In some aspects, the human is a juvenile or elderly human.

Some aspects of the present disclosure are directed to a composition for inducing trained immunity comprising a nanoparticle and a small molecule that consists of or comprises a glucocorticoid, A1155463, nerol, hydroquinone, 5-fluoroindole-2-carboxylic acid, myricetin, and fenoterol, a pharmaceutically acceptable salt thereof, and any combination thereof. In some aspects, the small molecule is encapsulated in the nanoparticle. The small molecule can be encapsulated within the nanoparticle, be dispersed throughout the nanoparticle, provided on the surface of the nanoparticle, or any combination thereof. In some aspects, the glucocorticoid consists of or comprises flunisolide, hydrocortisone, hydrocortisone acetate, halcinonide, budesonide, fluocinonide, fluocinolone acetonide, clobetasol propionate, prednisolone, triamcinolone, triamcinolone diacetate, nitrofurazone, and benseraside hydrochloride. Any one or more of the preceding glucocorticoids may be excluded in certain aspects. In some aspects, the nanoparticle comprises a biodegradable polymer. In some aspects, the biodegradable polymer consists of or comprises polyesters, polyamides, polyurethanes, polyureas, poly(amide-enamine)s, polyanhydrides, polyvinyl alcohols, polyacrylates, proteins, polysaccharides, natural rubber, and polyolefins. In some aspects, a polyolefin includes an additive to enhance biodegradation. In some aspects, the polyester is poly(lactic-co-glycolic acid).

In some aspects, a method as disclosed herein further comprises administering two or more of the small molecules to the subject. The first administered small molecule and optional second small molecule may be administered to the subject simultaneously. The first administered small molecule and the additional or second small molecule may be administered to the subject sequentially. The additional or second administered small molecule may be administered to the subject subsequent to administering the first small molecule. The additional or second administered small molecule may be administered to the subject at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 40, 44, 48, or 72 hours, or more, after administering the first small molecule to the subject. The additional or second administered small molecule may be administered to the subject at least 12 hours after administering the first small molecule to the subject. The additional or second administered small molecule may be administered to the subject at least 18 hours after administering the first small molecule to the subject. The additional or second administered small molecule may be administered to the subject at least 24 hours after administering the first small molecule to the subject. In some aspects, the additional or second administered small molecule is administered minutes to weeks, or any range derivable therein, after administration of the first small molecule. The additional or second administered small molecule may be administered to the subject prior to administering the first small molecule to the subject.

The concentration of the small molecule may be 0.1 μM-10 μM. The concentration of the small molecule may be, may be at least, or may be at most 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 pM, nM, μM, mM, or any range derivable therein.

In some aspects, the presently disclosed subject matter provides a method of inducing a non-specific, pro-inflammatory phenotype in an innate immune cell or organism, wherein the method comprises contacting the cell or organism with a small molecule inducer of trained immunity in an amount sufficient to induce a future pro-inflammatory response in the cell or organism, wherein the future pro-inflammatory response occurs when the cell or organism is exposed to an immunological challenge at a time point at least one week after said contacting; and further wherein said small molecule inducer of trained immunity is selected from the group comprising a glucocorticoid, A1155463, nerol, hydroquinone, 5-fluoroindole-2-carboxylic acid, myricetin, fenoterol, and pharmaceutically acceptable salts thereof. In some embodiments, the immunological challenge is selected from an infectious agent, a secondary metastasis, or a vaccine.

In some aspects, the presently disclosed subject matter provides a method of preventing an infection in a subject in need thereof, wherein the method comprises administering to the subject a small molecule inducer of trained immunity in an amount sufficient to induce trained immunity in the subject, wherein said administering is performed at least one week prior to contact by, or expected risk of contact by, the subject with an infectious agent, and wherein said small molecule inducer of trained immunity is selected from the group comprising a glucocorticoid, A1155463, nerol, hydroquinone, 5-fluoroindole-2-carboxylic acid, myricetin, fenoterol, and pharmaceutically acceptable salts thereof. In some embodiments, the subject in need thereof is an elderly subject, a juvenile subject, or an immunocompromised subject. In some embodiments, the administering is performed at least three weeks prior to contact by, or expected risk of contact by, the subject with an infectious agent.

In some aspects, the presently disclosed subject matter provides a method of reducing the risk of a secondary tumor in a subject diagnosed with a primary cancer, wherein the method comprises: (a) treating the subject with a standard regimen of cancer treatment specific for the primary cancer of said subject, wherein the standard regimen of treatment comprises chemotherapy, surgical removal of cancer cells, radiation therapy, immunotherapy, or a combination thereof; and (b) administering to the subject, at a time following completion of the treating of step (a), a small molecule inducer of trained immunity selected from the group comprising A1155462, a glucocorticoid, nerol, hydroquinone, 5-fluoroindole-2-carboxylic acid, myricetin, fenoterol and pharmaceutical acceptable salts thereof in an amount sufficient to induced trained immunity in the subject, thereby reducing the risk of secondary tumor in the subject compared to the risk of secondary tumor in a subject treated for a similar cancer and not administered the small molecule inducer of trained immunity.

In some aspects, the presently disclosed subject matter provides a method of increasing the efficacy of a vaccine in a subject in need thereof, wherein the method comprises administering to the subject a small molecule inducer of trained immunity in an amount sufficient to induce trained immunity in the subject, wherein said administrating is performed at least one week prior to administration of the vaccine to the subject, and wherein said small molecule inducer of trained immunity is selected from the group comprising a glucocorticoid, A1155463, nerol, hydroquinone, 5-fluoroindole-2-carboxylic acid, myricetin, and fenoterol, and pharmaceutically acceptable salts thereof. In some embodiments, the subject in need thereof is an elderly subject, a juvenile subject, or an immunocompromised subject. In some embodiments, said administering of the small molecule inducer of trained immunity is performed at least three weeks or at least four weeks prior to administration of the vaccine.

Accordingly, it is an object of the presently disclosed subject matter to provide methods of inducing a long-term, non-specific pro-inflammatory phenotype in a cell or organism, of preventing or reducing the risk of disease, e.g., secondary cancers or microbial infections in a subject in need thereof, and of increasing the efficacy of a vaccine in a subject in need thereof via administration of small molecule inducers of trained immunity.

Methods include but are not limited to method of inducing trained immunity in a subject, methods of preventing an infection in a subject, methods of improving an immune response in a subject, methods for reducing a risk of a secondary tumor in a subject diagnosed with a primary cancer, methods for increasing the efficacy of a vaccine administered to a subject, methods for increasing an immune response, methods for treating a patient with immunotherapy, methods of modulating an immune response, and methods of modulating at least one protein in the BCL pathway.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Any embodiment discussed with respect to one aspect of the disclosure applies to other aspects of the disclosure as well and vice versa. For example, any step in a method described herein can apply to any other method. Moreover, any method described herein may have an exclusion of any step or combination of steps. Aspects of an embodiment set forth in the Examples are also aspects that may be implemented in the context of aspects discussed elsewhere in a different Example or elsewhere in the application, such as in the Summary, Detailed Description, Claims, and Brief Description of the Drawings.

Other objects, features and advantages of the present invention will become apparent from the following detailed description. It should be understood, however, that the detailed description and the specific examples, while indicating specific aspects of the invention, are given by way of illustration only, since various changes and modifications within the spirit and scope of the invention will become apparent to those skilled in the art from this detailed description.

An object of the presently disclosed subject matter having been stated hereinabove, and which is achieved in whole or in part by the presently disclosed subject matter, other objects will become evident as the description proceeds when taken in connection with the accompanying drawings and examples as best described hereinbelow.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific aspects presented herein.

FIGS. 1A-1G: High-throughput screen for small molecule inducers of trained immunity. FIG. 1A shows a schematic diagram of an in vitro high throughput screening method for small molecule inducers of trained immunity with checkpoint measurements. FIG. 1B is a graph showing the tumor necrosis factor-alpha (TNF-α) concentration of all compounds screened represented as fold change over untrained control (n=3). The solid line at fold change=1.6 represents the cutoff for hit identification. FIG. 1C is a graph showing the classification of hit compounds represented as a percent out of 32 hits. FIG. 1D shows the TNF-α concentration fold change over untrained control of 7 hit compounds chosen for further evaluation. FIG. 1E shows the chemical structures of the 7 compounds from FIG. 1D chosen for further evaluation. FIG. 1F is a graph depicting BMDMs treated with IFN-γ 5 days following training produce negligible amounts of TNF-α in high-throughput screen. There was no significant difference between cells treated with only DMSO compared to those treated with any library compounds. FIG. 1G is a graph showing β-glucan as whole glucan particles (WGP) induces trained immunity responses. BMDMs trained with WGP exhibit a 1.7-fold increase in TNF-α production relative to untrained control in a high-throughput screening context. All values are expressed as mean±SEM, and statistics were conducted using 1-way ANOVA with Dunnett's multiple comparisons test (significance compared with untrained group ***P<0.001, ****P<0.0001).

FIGS. 2A-21: In vitro confirmation of screen hits. FIGS. 2A-2G are graphs showing the tumor necrosis factor-alpha (TNF-α) concentration (expressed in picograms per milliliter (pg/mL) from bone marrow-derived macrophages (BMDMs) trained with phosphate buffered saline (PBS) or hit compounds (FIG. 2A Flunisolide; FIG. 2B 5-Fluoroindole-2-carboxylic acid; FIG. 2C Myricetin; FIG. 2D Hydrocortisone; FIG. 2E Nerol; FIG. 2F Hydroquinone; FIG. 2G Fenoterol hydrobromide) at concentrations of 10 micromolar (μM)-100 nanomolar (nM) followed by a challenge with 100 nanograms per milliliter (ng/mL) PAM3-CSK4 (Pam3) (n=15).

FIG. 2H is a graph showing TNF-α concentration from BMDMs trained with PBS or hydrocortisone at a concentration of 10 μM followed by challenge with 100 nanograms per milliliter (ng/mL) lipopolysaccharide (LPS) or Pam3 (n=30). Training effects were observed with both toll-like receptor 4 (TLR4) and toll-like receptor 1/2 (TLR1/2) agonists. FIG. 2I is a graph showing TNF-α or interleukin-6 (IL-6) concentration from BMDMs treated with 10 μM hit compounds one hour (hr) prior to 24 hr stimulation with 100 ng/mL Pam3 (n=6). In an acute (nontraining) PAM3 challenge, glucocorticoids significantly suppressed TNF-α production. All values are expressed as mean±SEM, and statistics were conducted using 1-way ANOVA with Dunnett's multiple comparisons test (significance compared with untrained group). *P<0.05, **P<0.01, ***P<0.001, ****P<0.0001, n.s., not significant.

FIGS. 3A-3I: Chemical inhibition of trained immunity. FIG. 3 shows a series of three graphs of tumor necrosis factor-alpha (TNF-α) concentration (expressed in picograms per milliliter (pg/mL)) following PAM3-CSK4 (Pam3) challenge in cells trained with 10 micromolar (M) flunisolide after pre-treatment with phosphate buffered saline (PBS), 5′-deoxy-5′-(methylthio)adenosine (MTA, a histone methyl transferase inhibitor, FIG. 3A), (-)-epigallocatechin-3-gallate (EGCG, a histone acetyltransferase inhibitor, FIG. 3B) or 2′-dioxy-D-glucose (2DG, a glycolysis inhibitor, FIG. 3C) (n=15). FIG. 3D is a graph of a glycolysis inhibition assay using 1 mM 2DG and measuring TNF-α concentration following Pam3 challenge in cells trained with 10 μM hydrocortisone after 1 h of pre-treatment with PBS or 2DG (n=15). FIGS. 3E-3I are graphs of TNF-α concentration following Pam3 challenge in cells trained with M 5-Fluoroindole-2-Carboxylic Acid (FIG. 3E), Myricetin (FIG. 3F), Nerol (FIG. 3G), Hydroquinone (FIG. 3H), or Fenoterol hydrobromide (FIG. 3I) after pre-treatment with PBS or glycolysis inhibitor, 2DG (n=15). These small molecules were not susceptible to suppression of training via inhibition of glycolysis. All values are expressed as mean±SEM, and statistics were conducted using 2-way ANOVA with Sidak's multiple comparisons test (significance compared with untrained group). *P<0.05, **P<0.01, ***P<0.001, ****P<0.0001, n.s., not significant.

FIGS. 4A-4G: In vivo training with small molecules. FIG. 4A is a schematic drawing showing a method of screening small molecule inducers of trained immunity in vivo. Mice were injected intraperitoneally (i.p.) with small molecules or vehicle control on days 0, 2, and 4. Mice were challenged with 1 μg lipopolysaccharide (LPS) on day 11 and then bled 1 hour (h) later. FIG. 4B is a pair of graphs showing (left) the tumor necrosis factor alpha (TNF-α) or (right) interleukin-6 (IL-6) concentration from mice (measured in picograms per milliliter (pg/mL) trained with vehicle or small molecule inducers of training (n=10). All compounds were given at a dose of 1.5 micromoles (moles) except hydrocortisone and 5-fluoroindole-2-carboxylic acid, which were given at 2 μmoles. FIG. 4C is a graph showing that mice trained with myricetin exhibited significantly increased cell count in the peritoneal cavity 2 h after challenge. (n=8) FIG. 4D is a pair of graphs showing training with either flunisolide or myricetin resulted in significantly increased number of TNF-α⁺ (top) or IL6⁺ (bottom) eosinophils in the peritoneal lavage. FIG. 4E is a pair of graphs showing training with hydroquinone resulted in significantly higher MFI of TNF-α⁺ neutrophils (left) and IL6⁺ eosinophils (right) in the peritoneal lavage. FIG. 4F is a graph showing that mice trained with flunisolide had a significant increase in MFI of CD86⁺ eosinophils and SPMs. FIG. 4G is a graph showing that mice trained with flunisolide had a significant increase in MFI of MHCII⁺ SPMs. B-glucan trained mice had significant increase in MFI of MHCII⁺ eosinophils, neutrophils, and LPMs. All values are expressed as mean±SEM, and statistics were conducted using 1-way ANOVA or 2-way ANOVA with multiple comparisons (significance compared with untrained group). *P<0.05, **P<0.01, ***P<0.001, ****P<0.0001, n.s., not significant. All values are expressed as mean±SEM, and statistics were conducted using 1-way ANOVA with two-stage linear step-up procedure (significance compared with untrained group). *P<0.05, **P<0.01, ***P<0.001, ****P<0.0001, n.s., not significant.

FIGS. 5A-5F: Testing A1155463 as an inducer of trained immunity (TI) in vitro. FIG. 5A is a schematic diagram for in-vitro TI assay in bone marrow-derived macrophages (BMDMs). FIG. 5B is a graph showing interleukin-6 (IL-6) levels (expressed in picograms per milliliter (pg/ml) 24 hours (h) post PAM3-CSK4 (Pam3) challenge in an in-vitro TI assay in BMDMs trained with either phosphate buffered saline (PBS) (black) or A1155463 (pink); n=15. FIG. 5C is a graph showing tumor necrosis factor alpha (TNF-α) levels (pg/ml) 24 h post Pam3 challenge in an in-vitro TI assay in BMDMs trained with either PBS (black) or A1155463 (pink); n=10. FIG. 5D is a graph showing IL-6 levels (pg/ml) 24 h post-stimulation with PBS (black) or A1155463 (pink); n=3. FIG. 5E is a graph showing the fold change in TNF-α following Pam3 challenge in BMDMs trained with A1155463 pre-treated with PBS, MTA (histone methyl transferase inhibitor), EGCG (histone acetyltransferase inhibitor) or 2-DG (glycolysis inhibitor); n=15, significance compared with PBS control of each inhibitor. FIG. 5F is a graph of IL-6 levels (pg/ml) 24 h post Pam3 challenge in an in-vitro TI assay in BMDMs trained with either PBS (black) or A1155463 (pink) pretreated with varying concentrations of 2-DG; n=5. All values are expressed as mean±SEM, and statistics were conducted using unpaired student's T-test or 2-way ANOVA with Sidak's multiple comparisons test (significance compared with PBS group). *P<0.05, **P<0.01, ***P<0.001, ****P<0.0001, n.s., not significant.

FIGS. 6A-6H: in-vivo A1155463 trained immunity (TI) assays. FIG. 6A is a schematic showing the in-vivo training schedule. Mice were dosed intraperitoneally (i.p.) with training material (PBS or A1155463 at 15 milligrams per kilogram body weight (mg/kg)) daily for six days. Mice were challenged with 5 mg lipopolysaccharide (LPS) on day 7. FIG. 6B is a graph showing the interleukin-6 (IL-6) and tumor necrosis factor-alpha (TNF-α) levels (expressed as picograms per milliliter (pg/ml) 1 hour (h) following LPS challenge with either PBS (black) or A1155463 (pink); n=5. FIG. 6C is a graph showing the percentage (%) of small peritoneal macrophages (SPMs) from the peritoneal cavity of PBS (black) or A1155463 trained (pink) mice 2 h post LPS challenge on day 7. FIG. 6D is a graph showing the IL-6 levels (pg/ml) from bone-marrow cells isolated on day 7 challenged ex-vivo with LPS from either PBS (black) or A1155463 (pink) trained mice; n=3. FIG. 6E is a schematic showing the in-vivo training schedule for B16.F10 tumor challenge. FIG. 6F is a graph showing the tumor volumes (in cubic millimeters (mm3)) of mice trained with PBS (black) or A1155463 (pink) mice; n=8. FIG. 6G is a schematic of the in-vivo training schedule for B16.F10 tumor challenge with checkpoint therapy (CPT) administered on day 14 and 17. FIG. 6H is a graph showing tumor volumes (mm³) of mice trained with PBS (black; n=4) or A1155463 (pink; n=5).

FIG. 7: Testing immunogenicity of A115463 on BMDMs: BMDMs (100,000 cells per well) were incubated with PBS (red), different doses of A1155463 (blue 10 uM, green 1 uM or purple 0.1 uM) or different concentrations of DMSO, reflective of the A1155463 doses for 24 h. Supernatant was tested using a Legend plex mouse inflammation kit; n=10; All values are expressed as mean±SEM, and statistics were conducted using two-way ANOVA with Tukey's multiple comparisons test (significance compared as indicated). *P<0.05, **P<0.01, ***P<0.001, ****P<0.0001, n.s., not significant.

FIG. 8: RAW-Blue assay: RAW-Blue cells (100,000 cells per well) were incubated with PBS (black), A1155463 (pink) or 100 ng/mL LPS (green) for 18 h. Supernatant was tested using Quanti-Blue assay and absorbance measured at 620 nm; n=3; All values are expressed as mean±SEM, and statistics were conducted using one-way ANOVA with Dunnett's multiple comparisons test (significance compared with PBS group). *P<0.05, **P<0.01, ***P<0.001, ****P<0.0001, n.s., not significant.

FIGS. 9A-9E: Representative plots for Annexin V gating strategy for apoptotic cells: BMDMs were stimulated with nothing (unstained control, FIG. 9A), PBS (negative control, FIG. 9B), Camptothecin (positive control, FIG. 9C) or A1155463 (FIG. 9D); n=3, (except camptothecin n=1). Results from plots 9B-9D depicted in FIG. 9E. Statistics were conducted using one-way ANOVA with Dunnett's multiple comparisons test (significance compared with PBS group). *P<0.05, **P<0.01, ***P<0.001, ****P<0.0001, n.s., not significant.

FIG. 10: Ruling out innate immune priming: Mice were trained with PBS or vehicle (black) or A1155463 (pink) according to schedule illustrated in FIG. 6A. 24 h after the last dosing, mice were bled and serum analyzed for pro-inflammatory cytokines using cytokine bead array; n=3; statistics were conducted using one-way ANOVA with Sidak's multiple comparisons test (significance compared with PBS group). *P<0.05, **P<0.01, ***P<0.001, ****P<0.0001, n.s., not significant.

FIGS. 11A-11E: ATAC-seq identifies unique chromatin regulation in glucocorticoid-trained BMDMs. FIG. 11A is a schematic of BMDM training protocol for ATAC-seq library preparation. FIG. 11B is a plot demonstrating number of peaks with increased or decreased accessibility compared to untreated controls. (LFSR<0.05) FIG. 11C is a graph showing independent clustering of shared differentially accessible peaks by treatment shows that glucocorticoids share a unique profile compared to β-glucan or other small molecule-trained BMDMs. FIG. 11D is a diagram of a two-list enrichment analysis that identifies differentially accessible pathways shared between BMDMs trained by different stimuli. FIG. 11E is a diagram showing TF footprints and increased binding of KLF and Sp family transcription factors in j-glucan and non-glucocorticoid small molecule-trained BMDMs. Glucocorticoid-trained BMDMs also exhibited decreased Cebp TF binding.

FIGS. 12A-12P: FIGS. 12A-12H are plots of in vitro treatment of BMDMs with hit compounds results in differentially accessible chromatin. Significant differentially accessible peaks (LFSR<0.05) are shown in volcano plots as upregulated (right half of plot) or downregulated (left half of plot) for each compound. FIG. 12I is a plot showing distinct changes in chromatin accessibility between treatments. PCA from mashr betas showing β-glucan, glucocorticoids, and the remaining hit compounds clustering in separate areas of the multidimensional space. FIG. 12J is a diagram depicting an analysis of overlapping genes that identify shared pathways. Signaling by IL-4 and IL-13 is more accessible in BMDMs treated with each compound, whereas glucocorticoids uniquely increase accessibility of GPCR ligand binding and rhodopsin-like receptors. FIG. 12K is a graph showing the number of upregulated or downregulated TFs relative to PBS-treated control. Glucocorticoids result in downregulated TF binding, while other small molecules and β-glucan result in upregulated TF binding (Benjamini-Hochberg adjusted p<0.1). Example traces of KLF6 (FIG. 12L), CEBPG (FIG. 12M), TCFL5 (FIG. 12N), and NFκB (FIG. 12O) showing differential occupancy in footprinting analysis.

FIG. 12P is a diagram depicting differential accessibility of TF binding sites by treatment condition in trained BMDMs.

FIGS. 13A-13G: FIGS. 13A-13B Representative increases in IL-6 production. Small-molecule training results in increased production of IL-6 following challenge with 100 ng/mL Pam3 (FIG. 13A myricetin; FIG. 13B hydrocortisone). FIG. 13C For each compound except nerol, there is a significant positive dose response from 100 nM to 10 μM (p<0.0001). FIGS. 13D-13E Small molecules do not induce significant pro-inflammatory cytokine production. TNF-α (FIG. 13D) and IL-6 (FIG. 13E) were measured from BMDM cell culture supernatant 24 h following treatment with small molecules or β-glucan. FIGS. 13F-13G BMDMs treated with IFN-γ 5 days following training produce negligible amounts of proinflammatory cytokines. There was no significant difference between cells treated with only DMSO compared to those treated hit compounds or β-glucan.

FIGS. 14A-14B: Schematic representations of the proposed mechanism of action of PLGA nanoparticles encapsulating #-glucan. FIG. 14A Trained immunity mediated by free #l-glucan confers protective effects that last only one week. FIG. 14B Sustained release of #-glucan from engineered PLGA nanoparticles enable prolonged trained immunity effects that can last up to four weeks depending on the release profile.

FIGS. 15A-15G: Nanoparticle characterization and in vitro TI assays. FIG. 15A Scanning electron microscopy image of PLGA nanoparticles synthesized by double emulsion technique. (scale bar=100 nm). FIG. 15B 7 d in vitro release kinetics of encapsulated β-glucan from the synthesized PLGA nanoparticles at pH 7.4. FIG. 15C In vitro trained immunity assay comparing cytokine levels in resting cells or following an LPS challenge in BMDMs trained with either PBS, the free equivalent amount of β-glucan or PLGA nanoparticles encapsulating β-glucan, n=3, significance compared with PBS. FIG. 15D Assay determining changes in epigenetic training response. BMDMs were pretreated for 30 min with inhibitors targeting epigenetic modifications, namely MTA (histone methyltransferase inhibitor), EGCG (histone demethylase inhibitor), or pargyline hydrochloride (histone acetyltransferase inhibitor). Cytokine levels were measured after an LPS challenge on day 5 with BMDMs trained with PBS, the free equivalent amount of β-glucan, or PLGA nanoparticles n=3, significance compared with PBS (no inhibitor) group. FIG. 15E Assay determining changes in metabolic training response. BMDMs were pretreated for 30 min with inhibitors targeting metabolic pathways, namely wortmannin (Akt inhibitor), metformin (AMPK activator), rapamycin or 2-deoxy D-glucose (glycolysis inhibitor). Cytokine levels were measured after an LPS challenge on day 5 with BMDMs trained with PBS, the free equivalent amount of β-glucan, or PLGA nanoparticles, n=3, significance compared with PBS (no inhibitor) group. FIG. 15F BMDMs were treated with either free FITC-labeled β-glucan or nanoparticles encapsulating FITC-β-glucan for 24 h. BMDMs were analyzed by flow cytometry to measure the percentage of cells that were FITC+. Statistics was conducted using an unpaired student's T-test. FIG. 15G BMDMs were pretreated for 30 min with inhibitors targeting endocytosis pathways, namely dynasore (caveolae and clathrin inhibitor), filipin (caveolae inhibitor), or chlorpromazine (clathrin inhibitor). IL-6 levels were measured after an LPS challenge on day 5 with BMDMs trained with—PBS, the free equivalent amount of β-glucan, or PLGA nanoparticles, n=3, significance compared with PBS (no inhibitor) group. All values are expressed as mean±SEM. n=3 and statistics were conducted using a one or two-way ANOVA with Dunnett's multiple comparisons test (significance compared with PBS group) or student's T test. *P<0.05, **P<0.01, ***P<0.001, ****P<0.0001, n.s., not significant.

FIGS. 16A-16I: Altering the temporal response of trained immunity in vivo results in changes in trained macrophage populations. FIG. 16A In vivo trained immunity schematic: Training materials were administered on day 0 and day four, and separate sets of mice were challenged 7 or 28 d later. Systemic cytokines were measured 1 h after the LPS challenge. FIG. 16B Systemic IL-6 levels 1 h after LPS challenge on day 7 and day 28 with mice trained with PBS, the free equivalent amount of β-glucan, 1 mg of β-glucan or nanoparticles, n=10, significance compared with PBS (untrained) group. FIG. 16C In vivo biodistribution assay of NIR-labeled PLGA nanoparticles encapsulating β-glucan at 30 min and 13 d measured by IVIS, n=3. FIG. 16D PLGA degradation profile in vivo measured by fluorescence intensity over time. Data were fitted to one-phase decay characteristic of reported PLGA degradation kinetics. FIG. 16E Analysis of trained immunity phenotype in peritoneal macrophages isolated from mice 28 d after training. Training was administered on day 0 and day 4 with PBS, the equivalent amount of 3-glucan, 1 mg of β-glucan, or nanoparticles. After 28 d, peritoneal macrophages were harvested and challenged ex vivo with LPS (10 ng mL-1), and IL-6 levels were quantified, n=5. FIG. 16F Analysis of the effect of adoptive transfer of peritoneal macrophages from trained mice in conferring trained immunity phenotype in naïve mice. Training was administered on day 0 and day 4 with PBS, the equivalent amount of β-glucan, 1 mg of β-glucan, or nanoparticles. After 28 d, peritoneal macrophages were harvested and cultured for 1 d. 1 million peritoneal macrophages per group were adoptively transferred (i.p) to naïve mice. After 24 h, these mice were challenged with LPS, and serum cytokines were analyzed 3 h post-challenge; n=4. FIG. 16G Analysis of percentage of SPMs in the peritoneal cavity after LPS challenge after 7 d of training. Training was administered on day 0 and day 4 with PBS, the equivalent amount of β-glucan, 1 mg of β-glucan, or nanoparticles. After one week, mice were challenged with LPS, and peritoneal cells were harvested 2 h later. The percentage of small peritoneal macrophages (SPMs) (CD11b+F4/80−) was analyzed by flow cytometry, n=5. FIG. 16H Analysis of trained immunity phenotype in peritoneal macrophages after 7 d. Training was administered on day 0 and day 4 with PBS, the equivalent amount of β-glucan, 1 mg of β-glucan, 1 mg only on day 0, or 0.5 mg nanoparticles on both day 0 and day 4. Peritoneal macrophages were harvested on day 7 and challenged ex-vivo with LPS. Cell supernatants were analyzed for inflammatory cytokines-IL-6, n=5, significance compared with PBS (untrained) group. FIG. 16I Analysis of trained immunity phenotype in splenic macrophages after 7 d. Training was administered on day 0 and day 4 with PBS, the equivalent amount of β-glucan, 1 mg of β-glucan, 1 mg only on day 0, or 0.5 mg nanoparticles on both day 0 and day 4. Splenic macrophages were harvested and challenged ex-vivo with LPS. Cell supernatants were analyzed for inflammatory cytokines—IL-6. n=5, significance compared with PBS (untrained) group. All values are expressed as mean±SEM. n=3-5, Statistics was conducted using one or two-way ANOVA with Dunnett's multiple comparison testing to compare groups to PBS group or Tukey's multiple comparison testing to compare all groups as indicated. *P<0.05, **P<0.01, ***P<0.001, ****P<0.0001, n.s., not significant.

FIGS. 17A-17E Tumor challenge. FIG. 17A Experimental scheme for B16.F10 tumor challenge. FIG. 17B Mice were trained on day 0 and day 4 with the indicated training materials-PBS, the free equivalent amount of β-glucan, 1 mg β-glucan, or PLGA nanoparticles encapsulating β-glucan. Mice were then challenged subcutaneously with B16.F10 tumor cells. Tumor volume was recorded after the tumors were palpable-day 29 onwards. FIG. 17C Tumors were excised at the end of the experiment on day 46, and the percentage of myeloid cells (Cd45+Cd11b+) was analyzed using flow cytometry. FIG. 17D Tumors were excised at the end of the experiment on day 46, and the percentage of neutrophils (Cd45+Cd11b+Ly6g+) was analyzed using flow cytometry, significance compared to the PBS group. FIG. 17E Tumors were excised at the end of the experiment on day 46, and the percentage of macrophages (Cd45+Cd11b+Ly6g-F4/80+) was analyzed using flow cytometry, significance compared to the PBS group. Statistics was conducted using one-way ANOVA with Dunnett's multiple comparison testing to compare groups as indicated. All values are expressed as mean±SEM. n=5,*P<0.05, **P<0.01, ***P<0.001, ****P<0.0001, n.s., not significant.

FIGS. 18A-18C: Modulating PLGA properties for temporal control of trained immunity. FIG. 18A 7 d in vitro release profile of low molecular weight ester-terminated PLGA nanoparticles, high molecular weight acid-terminated nanoparticles, or high molecular weight ester-terminated nanoparticles at pH 7.4, n=3. FIG. 18B In vivo experimental protocol. FIG. 18C Mice were trained with either PBS low molecular weight acid-terminated PLGA nanoparticles, low molecular weight ester-terminated PLGA nanoparticles, high molecular weight acid-terminated nanoparticles, or high molecular weight ester-terminated nanoparticles on day 0. Different sets of mice (n=5) were challenged with LPS on the indicated days (7, 21, or 25), and systemic cytokines were quantified. Significance compared with PBS group. All values are expressed as mean±SEM. n=3-5 and statistics were conducted using a two-way ANOVA with Dunnett's multiple comparisons test,*P<0.05 and **P<0.01.

### DETAILED DESCRIPTION

According to aspects of the presently disclosed subject matter, alternative small molecule drugs have been identified that induce trained immunity. These small molecule inducers of trained immunity can increase understanding of trained immunity and broaden the scope of its applications, e.g., as they include molecules that do not induce acute cytokine responses and are not derived from pathogenic sources. More particularly, according to the presently disclosed subject matter and as described hereinbelow, more than two dozen small molecules were identified in several chemical classes that induce trained immunity in the absence of initial immune activation. As further described hereinbelow, several of these small molecules were used to characterize and establish training activity in vivo.

The presently disclosed subject matter will now be described more fully hereinafter with reference to the accompanying Examples, in which representative embodiments are shown. The presently disclosed subject matter can, however, be embodied in different forms and should not be construed as limited to the embodiments set forth herein. Rather, these embodiments are provided so that this disclosure will be thorough and complete, and will fully convey the scope of the embodiments to those skilled in the art.

Unless otherwise defined, all technical and scientific terms used herein have the same meaning as commonly understood by one of ordinary skill in the art to which this presently described subject matter belongs. Although any methods, devices, and materials similar or equivalent to those described herein can be used in the practice or testing of the presently disclosed subject matter, representative methods, devices, and materials are now described. All publications, patent applications, patents, and other references mentioned herein are incorporated by reference in their entirety.

Throughout the specification and claims, a given chemical formula or name shall encompass all optical and stereoisomers, as well as racemic mixtures where such isomers and mixtures exist, unless indicated otherwise.

### I. Definitions

While the following terms are believed to be well understood by one of ordinary skill in the art, the following definitions are set forth to facilitate explanation of the presently disclosed subject matter.

Following long-standing patent law convention, the terms “a”, “an”, and “the” refer to “one or more” when used in this application, including the claims. Thus, for example, reference to “a nanoparticle” includes a plurality of such nanoparticles, and so forth.

Unless otherwise indicated, all numbers expressing quantities of size, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about”, when referring to a value or to an amount of size (i.e., diameter), weight, concentration or percentage is meant to encompass variations of in one example ±20% or ±10%, in another example ±5%, in another example ±1%, and in still another example ±0.1% from the specified amount, as such variations are appropriate to perform the disclosed methods.

Numerical ranges recited herein by endpoints include all numbers and fractions subsumed within that range (e.g. 1 to 5 includes, but is not limited to, 1, 1.5, 2, 2.75, 3, 3.90, 4, and 5).

As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D.

The term “comprising”, which is synonymous with “including,” “containing,” or “characterized by” is inclusive or open-ended and does not exclude additional, unrecited elements or method steps. “Comprising” is a term of art used in claim language which means that the named elements are present, but other elements can be added and still form a construct or method within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms. The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention.

The term “small molecule” as used herein refers to a non-polymeric or oligomeric compound having a molecular weight of less than about 1000 Daltons. In some aspects, the small molecule has a molecular weight of less than about 900 Daltons, less than about 800 Daltons, or less than about 700 Daltons. Small molecules can be natural products or synthetic compounds.

The terms “nanoscale particle” and “nanoparticle” refer to a structure having at least one region with a dimension (e.g., length, width, diameter, etc.) of less than about 2000 nm. In some aspects, the dimension is about 1200 nm or less (e.g., less than about 1200 nm, less than about 1000 nm, less than about 900 nm, less than about 800 nm, less than about 750 nm, less than about 700 nm, less than about 650 nm or less than about 600 nm).

In some aspects, the nanoparticle is approximately spherical. When the nanoparticle is approximately spherical, the characteristic dimension can correspond to the diameter of the sphere. In addition to spherical shapes, the nanomaterial can be disc-shaped, plate-shaped (e.g., hexagonally plate-like), oblong, polyhedral, rod-shaped, cubic, or irregularly-shaped.

The terms “polymer” and “polymeric” refer to chemical structures that have repeating units (i.e., multiple copies of a given chemical substructure). Polymers can be formed from polymerizable monomers. A polymerizable monomer is a molecule that comprises one or more moieties that can react to form bonds (e.g., covalent or coordination bonds) with moieties on other molecules of polymerizable monomer. In some aspects, each polymerizable monomer molecule can bond to two or more other molecules/moieties. In some cases, a polymerizable monomer will bond to only one other molecule, forming a terminus of the polymeric material.

Polymers can be organic, or inorganic, or a combination thereof. As used herein, the term “inorganic” refers to a compound or composition that contains at least some atoms other than carbon, hydrogen, nitrogen, oxygen, sulfur, phosphorous, or one of the halides. Thus, for example, an inorganic compound or composition can contain one or more silicon atoms and/or one or more metal atoms.

As used herein “organic polymers” are those that do not include silica or metal atoms in their repeating units. Exemplary organic polymers include polyvinylpyrrolidone (PVO), polyesters, polyamides, polyethers, polydienes, and the like. Some organic polymers contain biodegradable linkages, such as esters, glycosidic bonds, or amides, such that they can degrade overtime under biological conditions.

“Biodegradable” means materials that are broken down or decomposed by natural biological processes. Biodegradable materials can be broken down for example, by cellular machinery, proteins, enzymes, hydrolyzing chemicals or reducing agents present in biological fluids or soil, intracellular constituents, and the like, into components that can be either reused or disposed of without significant toxic effect on the environment. Thus, the term “biodegradable” as used herein refers to both enzymatic and non-enzymatic breakdown or degradation of polymeric structures. In some aspects, the degradation time is a function of polymer composition and morphology. Suitable degradation times are from hours or days to weeks to years.

Exemplary biodegradable polymers include, but are not limited to, proteins (e.g., whey protein, casein), polysaccharides (e.g., starch), polylactic acid (PLA), poly(glycolic acid) (PGA), poly(lactic-co-glycolic acid) (PLGA), poly(hydroxybutyrate-co-hydroxyvalerate) (PHBV), poly(caprolactone) (PCL), poly(hydroxybutyrate) (PHB), polyhydroxyalkanoate (PHA), nylon-2-nylon-6 (i.e., a copolymer of glycine and aminocaproic acid), and the like.

The phrases “pharmaceutically acceptable” or “pharmacologically acceptable” refer to molecular entities and compositions that do not produce an adverse, allergic, or other untoward reaction when administered to an animal or human. As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the U.S. Federal government or listed in the U.S. Pharmacopeia for use in an animal. In some aspects, a pharmaceutically acceptable carrier is pharmaceutically acceptable for use in a human.

As used herein, an “effective amount” or “therapeutically effective amount” refers to an amount of a compound or composition sufficient to produce a selected effect, such as but not limited to inducing trained immunity. In the context of administering compounds in the form of a combination, such as multiple compounds, the amount of each compound, when administered in combination with one or more other compounds, can be different from when that compound is administered alone. Thus, an effective amount of a combination of compounds refers collectively to the combination as a whole, although the actual amounts of each compound may vary. The term “more effective” means that the selected effect occurs to a greater extent by one treatment relative to the second treatment to which it is being compared.

The term “prevent”, as used herein, means to stop something from happening, or taking advance measures against something possible or probable from happening. In the context of medicine, “prevention” generally refers to action taken to decrease the chance of getting a disease or condition. It is noted that “prevention” need not be absolute, and thus can occur as a matter of degree.

A “preventive” or “prophylactic” treatment is a treatment administered to a subject who does not exhibit signs, or exhibits only early signs, of a condition, disease, or disorder. A prophylactic or preventative treatment is administered for the purpose of decreasing the risk of developing pathology associated with developing the condition, disease, or disorder.

“Individual, “subject,” and “patient” are used interchangeably and can refer to a human or non-human. As used herein, a “subject in need thereof” is a patient, animal, mammal, or human, who will benefit from the method of this presently disclosed subject matter. The term “immunocompromised” refers to a weakened immune system.

Immunocompromised subjects include, but are not limited to, those with certain diseases that result in immune deficiency (e.g., advanced HIV infection or severe combined immunodeficiency), those with a hematologic malignancy (e.g., leukemia), those who are being treated with an immunosuppressive therapy (e.g., to prepare for organ transplant), or who are actively being treated for a solid or hematologic malignancy and/or who are taking certain medications that result in a suppressed immune system (e.g., antimetabolites, alkylating agents, etc.).

The term “juvenile” as used herein varies by species. In a human, for example, “juvenile” can refer to a subject who is younger than about 21 (e.g., human up to the age of about 18, up to the age of about 14, or up to the age of about 12).

Similarly, the term “elderly” as used herein can vary by species. In a human, for example, “elderly” can refer to a subject over the age of about 65. In some aspects, an elderly patient is one who is at least or at most 60, 61, 62, 63, 64, 65, 66, 67, 68. 69, 70, 71, 72, 73, 74, 75, 76, 77, 78, 79, 80, 81, 82, 83, 84, 85, 86, 87, 88, 89, 90 years old or more (or any range derivable therein).

The term “cancer” as used herein refers to diseases caused by uncontrolled cell division and/or the ability of cells to metastasize, or to establish new growth in additional sites. The terms “malignant”, “malignancy”, “neoplasm”, “tumor,” “cancer” and variations thereof refer to cancerous cells or groups of cancerous cells.

Particular types of cancer include, but are not limited to, skin cancers (e.g., melanoma), connective tissue cancers (e.g., sarcomas), adipose cancers, breast cancers, head and neck cancers, lung cancers (e.g., mesothelioma), stomach cancers, pancreatic cancers, ovarian cancers, cervical cancers, uterine cancers, anogenital cancers (e.g., testicular cancer), kidney cancers, bladder cancers, colon cancers, prostate cancers, central nervous system (CNS) cancers, retinal cancer, blood, neuroblastomas, multiple myeloma, and lymphoid cancers (e.g., Hodgkin's and non-Hodgkin's lymphomas).

The terms “primary cancer” or “primary tumor” refer to cancerous cells at an initial cancer site, while “secondary cancer” or “secondary tumor” refer to cancerous cells that are the result of metastasis of a primary cancer, i.e., are at a secondary site in the body, but which includes cancerous cells similar to cells from the primary cancer).

As used herein, the term “glucocorticoid” (“GC”) includes any compound known in the art that binds to and activates a glucocorticoid receptor. A GC is thus a glucocorticoid receptor agonist. Other terms for GC include corticoid, corticosteroid, steroid, and glucocorticosteroid. “Glucocorticosteroid” refers to a steroid hormone or steroidal molecule that binds to the glucocorticoid receptor. In humans and many other mammals, the primary GC is cortisol; however, in rodents, for example, corticosterone plays that role. Other GCs include, for example, but are not limited to, dexamethasone, prednisone, prednisolone, triamcinolone, hydrocortisone, beclamethasone, and other natural and synthetic compounds. Glucocorticosteroids are typically characterized by having 21 carbon atoms, an α,β-unsaturated ketone in ring A, and an α-ketol group attached to ring D. They differ in the extent of oxygenation or hydroxylation at C-11, C-17 and C-19 (see Rawn, “Biosynthesis and Transport of Membrane Lipids and Formation of Cholesterol Derivatives,” in Biochemistry, Daisy et al. (eds.), 1989, pg. 567).

The term “innate immune cell” as used herein refers to white blood cells that mediate innate immunity. Exemplary innate immune cells include, for example, basophils, dendritic cells, eosinophils, Langerhans cells, mast cells, monocytes, macrophages, neutrophils, and Natural Killer (NK) cells.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

### II. Nanoparticles

Certain aspects disclosed herein concern compositions comprising a nanoparticle, which may encapsulate a therapeutic agent, which can be any of the small molecules disclosed herein. In several aspects, as disclosed elsewhere herein, a nanoparticle composition (e.g., a mixed micelle composition, a liposomal composition, solid lipid particles, oil-in-water emulsions, water-in-oil-in-water emulsions, water-in-oil emulsions, oil-in-water-in-oil emulsions, etc.) is provided to aid in the delivery of a small molecule as disclosed herein. As disclosed elsewhere herein, in several aspects, the nanoparticles comprise a glucocorticoid, A1155463, nerol, hydroquinone, 5-fluoroindole-2-carboxylic acid, myricetin, fenoterol, a pharmaceutically acceptable salt thereof, and any combination thereof. Any one or more of the preceding small molecules may be excluded in certain aspects. In several aspects, a composition comprising the nanoparticles disclosed herein comprises a therapeutically effective amount of one or more therapeutic agents.

In several aspects, when formulated, the dry weight % of one or more therapeutic agents present in the nanoparticle compositions is equal to or at least about: 0.1%, 0.5%, 1%, 2.5%, 5%, 7.5%, 10%, 12.5%, 15%, 20%, 22.5%, 25%, 27.5%, 30%, 32.5%, 35%, 37.5%, 40%, 42.5%, 45%, 47.5%, 50%, 52.5%, 55%, 57.5%, 60%, or any range derivable therein. In several aspects, the therapeutic agents are provided in an aqueous composition. In several aspects, the wet weight % of one or more therapeutic agents present in the composition (with water included) is equal to or at least about: 0.5%, 1%, 2.5%, 5%, 7.5%, 10%, 12.5%, 15%, 20%, 22.5%, 25%, 27.5%, 30%, or any range derivable therein. In several aspects, the one or more therapeutic agents may be provided in the wet composition at a concentration of greater than or equal to about: 0.001 mg/mL, 0.005 mg/mL, 0.01 mg/mL, 0.05 mg/mL, 0.1 mg/mL, 0.5 mg/mL, 1 mg/mL, 5 mg/mL, 20 mg/mL, 30 mg/mL, 50 mg/mL, 100 mg/mL, or any range derivable therein.

In several aspects, the therapeutic agents, collectively or individually, are present in the aqueous nanoparticle composition at a concentration of less than or equal to about: 150 mg/mL, 100 mg/mL, 75 mg/mL, 50 mg/mL, 25 mg/mL, 20 mg/mL, 10 mg/mL, 5 mg/mL, 2.5 mg/mL, 2 mg/mL, 1.5 mg/mL, 1 mg/mL, 0.5 mg/mL, 0.1 mg/mL, 0.05 mg/mL, 0.01 mg/mL, or ranges including and/or spanning the aforementioned values. In several aspects, the one or more therapeutic agents, collectively or individually, are present in the aqueous composition at a concentration of greater than or equal to about: 150 mg/mL, 100 mg/mL, 75 mg/mL, 50 mg/mL, 25 mg/mL, 20 mg/mL, 10 mg/mL, 5 mg/mL, 2.5 mg/mL, 2 mg/mL, 1.5 mg/mL, 1 mg/mL, 0.5 mg/mL, 0.1 mg/mL, 0.05 mg/mL, 0.01 mg/mL, or ranges including and/or spanning the aforementioned values. In several aspects, the one or more therapeutic agents, collectively or individually, are present in the composition at a dry wt. % of equal to or at least about: 0.1%, 0.5%, 1%, 2.5%, 5%, 7.5%, 10%, 12.5%, 15%, 20%, 22.5%, 25%, 27.5%, 30%, 32.5%, 35%, 37.5%, 40%, 42.5%, 45%, 47.5%, 50%, 52.5%, 55%, 57.5%, 60%, or ranges including and/or spanning the aforementioned values. In several aspects, the one or more therapeutic agents, collectively or individually, are present in the composition at a wet wt. % of equal to or at least about: 0.1%, 0.5%, 1%, 2.5%, 5%, 7.5%, 10%, 12.5%, 15%, 20%, 22.5%, 25%, 27.5%, 30%, 32.5%, 35%, 37.5%, 40%, 42.5%, 45%, 47.5%, 50%, 52.5%, 55%, 57.5%, 60%, or ranges including and/or spanning the aforementioned values. In several aspects, as disclosed elsewhere herein, the composition is aqueous, while in others it has been dried into a powder (that is free of or substantially free of water). In several aspects, where the composition has been dried, it comprises a water content of less than or equal to 20%, 15%, 10%, 7.5%, 5%, 2.5%, 1%, or ranges including and/or spanning the aforementioned values.

In several aspects, as disclosed elsewhere herein, the composition is aqueous (e.g., contains water) while in other aspects, the composition is dry (lacks water or substantially lacks water). In several aspects, the composition has been dried (e.g., has been subjected to a process to remove most or substantially all water). In several aspects, the composition comprises nanoparticles in water (e.g., as a solution, suspension, or emulsion). In other aspects, the composition is provided as a powder (e.g., that may be constituted or reconstituted in water). In several aspects, as disclosed elsewhere herein, the water content (in wt. %) of the composition is less than or equal to about: 30%, 20%, 10%, 5%, 2.5%, 1%, 0.5%, 0.1%, 0%, or ranges including and/or spanning the aforementioned values. In several aspects, as disclosed elsewhere herein, the water content (in wt. %) of the composition is greater than or equal to about: 50%, 60%, 70%, 80%, 85%, 90%, 92.5%, 95%, 97.5%, or ranges including and/or spanning the aforementioned values. In several aspects, the water is nanopure, deionized, USP grade, WFI, and/or combinations of the foregoing. In some aspects, the composition is a dried composition comprising a nanoparticle having weight ratios of a first therapeutic agent: a nanoscale coordination polymer NCP: optionally a lipid source, and optionally a surfactant of 1 to 50:1 to 50:1 to 50:0 to 17.5.

In several aspects, as disclosed elsewhere herein, the nanoparticle composition provides an oil-in-water emulsion (e.g., a nanoemulsion), water-in-oil emulsion, a water-in-oil-in-water emulsion, an oil-in-water-in-oil emulsion, a liposome (and variants including multi-lamellar, double liposome preparations, etc.), micelle, and/or solid lipid particles. Any one of these structures may be provided as a nanoparticle or microparticle.

As disclosed elsewhere herein, in some aspects, the nanoparticle composition comprises a lipid source. In several aspects, the lipid source comprises a charged lipid, which can impart a charge to the nanoparticle. In several aspects, the lipid source comprises a neutral lipid. In several aspects, the lipid source comprises one or more phospholipids. In several aspects, the one or more phospholipids comprises one or more of phosphatidic acid, phosphatidylethanolamine, phosphatidylcholine, phosphatidylserine, phosphatidylinositol, phosphatidylinositol phosphate, phosphatidylinositol bisphosphate, phosphatidylinositol trisphosphate, lipoid H 100-3, phospholipon 90H, phospholipon 80H, lipoid 100-3, lipoid P75-3, 1,2-dioleoyl-sn-glycero-3-phosphate (DOPA), 1,2-dioleoyl-sn-glycero-3-phosphocholine (DOPC), 1,2-diastearoyl-sn-glycero-3-phosphoethanolamine-N-[amino(polyethylene glycol)2000] (DSPE-PEG2000) or any combination of the foregoing. In several aspects, the lipid source is a phosphatidylcholine. In several aspects, the one or more lipid source lipid(s) (collectively or individually) are present in the composition at a dry wt. % of equal to or less than about: 0%, 5%, 10%, 15%, 20%, 30%, 40%, 50%, 60%, or ranges including and/or spanning the aforementioned values. In several aspects, the one or more lipid source lipid(s) (collectively or individually) are present in the composition at a wet wt. % of equal to or less than about: 0%, 0.1%, 0.5%, 1.0%, 2.5%, 4%, 5%, 6%, 7.5%, 10%, 12.5%, 15%, 17.5%, 20%, 25%, 30%, 35%, 40%, 45%, 50%, or ranges including and/or spanning the aforementioned values. In several aspects, the one or more lipid source lipid(s) (collectively or individually) are present in the composition at a wet w/v of equal to or less than about: 0 mg/mL, 0.1 mg/mL, 0.5 mg/mL, 1.0 mg/mL, 2.5 mg/mL, 4 mg/mL, 5 mg/mL, 6 mg/mL, 7.5 mg/mL, 10 mg/mL, 12.5 mg/mL, 15 mg/mL, 17.5 mg/mL, 20 mg/mL, 25 mg/mL, 30 mg/mL, 35 mg/mL, 40 mg/mL, 45 mg/mL, 50 mg/mL, or ranges including and/or spanning the aforementioned values. In several aspects, as disclosed elsewhere herein, the composition is aqueous, while in others it has been dried into a powder. For instance, as disclosed elsewhere herein, in several aspects, the composition is aqueous (wet), while in others it has been dried into a powder (dry). In several aspects, the one or more lipid(s) of the lipid source are synthetic, derived from sunflower, soy, egg, or mixtures thereof. In several aspects, the one or more lipids of the lipid source can be hydrogenated or non-hydrogenated. In several aspects, the lipid source exceeds requirements of the United States Pharmacopeia (is USP grade) and/or is National Formulary (NF) grade.

In several aspects, the one or more lipids of the lipid source has a purity of greater than or equal to about: 92.5%, 95%, 96%, 96.3%, 98%, 99%, 100%, or ranges including and/or spanning the aforementioned values. In several aspects, the one or more lipids of the lipid source has a total % impurity content by weight of less than or equal to about: 8.5%, 5%, 4%, 3.7%, 2%, 1%, 0%, or ranges including and/or spanning the aforementioned values.

As disclosed elsewhere herein, in some aspects, the nanoparticle composition comprises a surfactant. In certain aspects, the nanoparticle composition does not comprise a surfactant. In several aspects, the surfactant is a pharmaceutically acceptable surfactant. In several aspects, the surfactant is a food surfactant. In several aspects, the surfactant comprises one or more of a polyoxyethylene sorbitan esters (e.g., polysorbates/tweens, including polysorbate 80, polysorbate 20, etc.), cremophor (e.g., a non-ionic solubilizer and emulsifier that is made by reacting ethylene oxide with castor oil), propylene oxide-modified polymethylsiloxane, dodecyl betaine, lauramidopropyl betaine, cocoamido-2-hydroxypropyl sulfobetaine, sodium stearate (or other stearate salts), polyoxyethylene alcohol, lecithins, mono- and diglycerides of fatty acids (MDG), acetic acid esters of MDG, lactic acid esters of MDG, citric acid esters of MDG, mono- and diacetyl tartaric acid esters of MDG, sucrose esters of fatty acids, polyglycerol esters of fatty acids (e.g., polyglycerol esters), polyglycerol polyricinoleate, propane-1,2-diol esters of fatty acids, propylene glycol esters, sodium stearoyl-2-lactylate, calcium stearoyl-2-lactylate, sorbitan fatty acid esters, quillaja extract surfactant, yucca extract surfactant, saponins, silicone emulsifiers, sorbitan trioleate, soya lecithin, dioctyl sodium sulfosuccinate, dioctyl sodium sulfonate, polyoxyethylene, hydrogenated castor oil, sucrose fatty acid ester, or combinations of any of the foregoing. Natural or synthetic surfactants can be used, including polyethylene glycol and dextrans, such as cyclodextran. In several aspects, the one or more surfactants are present in the nanoparticle composition (collectively or individually) at a dry wt. % of equal to or less than about: 0%, 5%, 10%, 15%, 20%, 30%, 40%, 50%, 60%, or ranges including and/or spanning the aforementioned values. Surfactants can include cationic, anionic, non-ionic, and zwitterionic surfactants. In several aspects, the one or more surfactants (collectively or individually) are present in the composition at a wet wt. % of equal to or less than about: 0%, 0.1%, 0.5%, 1.0%, 2.5%, 4%, 5%, 6%, 7.5%, 10%, 12.5%, 15%, 17.5%, or ranges including and/or spanning the aforementioned values. In several aspects, the one or more surfactants (collectively or individually) are present in the composition at a wet w/v of equal to or less than about: 0 mg/mL, 0.1 mg/mL, 0.5 mg/mL, 1.0 mg/mL, 2.5 mg/mL, 4 mg/mL, 5 mg/mL, 6 mg/mL, 7.5 mg/mL, 10 mg/mL, 12.5 mg/mL, 15 mg/mL, 17.5 mg/mL, or ranges including and/or spanning the aforementioned values. In several aspects, the surfactant exceeds requirements of the United States Pharmacopeia (is USP grade) and/or is National Formulary (NF) grade.

In several aspects, one or more co-emulsifiers are used. In certain aspects, the nanoparticle composition does not comprise a co-emulsifer. In several aspects, the co-emulsifier is a pharmaceutically acceptable co-emulsifier. In several aspects, the co-emulsifier consists of or comprises oleic acid, miglyol 812N (all versions), cetearyl olivate, isoprpyle myristate, celluloses, polysaccharides (e.g., methylcellulose, propylmethylcellulose, hydroxypropyl methylcellulose, xanthan gum, etc.), capric acid, caprylic acid, triglycerides (e.g., triglycerides of oleic acid, capric acid, caprylic acid (Captex 8000, Captex GTO, Captex 1000)), glycerol monooleate, glyceryl stearate, glycerol monostearate (Geleol™ Mono and Diglyceride NF), omega-3 fatty acids (α-linolenic acid (ALA), eicosapentaenoic acid (EPA), docosahexaenoic acid (DHA), Tonalin, Pronova Pure® 46:38, free fatty acid Tonalin FFA 80), conjugated linoleic acid (CLA), alpha glycerylphosphorylcholine (alpha GPC), palmitoylethanolamide (PEA), cetyl alcohol, or emulsifying wax and/or combinations of any of the foregoing. In several aspects, the one or more co-emulsifiers are present in the nanoparticle composition (collectively or individually) at a dry wt. % of equal to or less than about: 0%, 5%, 10%, 15%, 20%, 30%, 40%, 50%, 60%, or ranges including and/or spanning the aforementioned values. In several aspects, the one or more co-emulsifiers (collectively or individually) are present in the composition at a wet wt. % of equal to or less than about: 0%, 0.1%, 0.5%, 1.0%, 2.5%, 4%, 5%, 6%, 7.5%, 10%, 12.5%, 15%, 17.5%, or ranges including and/or spanning the aforementioned values. In several aspects, the one or more co-emulsifiers (collectively or individually) are present in the composition at a wet w/v of equal to or less than about: 0 mg/mL, 0.1 mg/mL, 0.5 mg/mL, 1.0 mg/mL, 2.5 mg/mL, 4 mg/mL, 5 mg/mL, 6 mg/mL, 7.5 mg/mL, 10 mg/mL, 12.5 mg/mL, 15 mg/mL, 17.5 mg/mL, or ranges including and/or spanning the aforementioned values. In several aspects, the co-emulsifiers exceeds requirements of the United States Pharmacopeia (is USP grade) and/or is National Formulary (NF) grade. In some aspects, the co-emulsifier component comprises a medium chain triglyceride (MCT). In some aspects, the medium chain triglyceride comprises a fatty acid selected from one or more of caprioc acid, octanoic acid, capric acid, caprylic acid, and/or lauric acid (e.g., is formed from). In some aspects, the medium chain triglyceride comprises a fatty acid 6-12 carbons in length (e.g., 6, 7, 8, 9, 10, 11, or 12). In some aspects, the co-emulsifier component comprises a long chain triglyceride (LCT). In some aspects, the long chain triglyceride comprises a fatty acid greater than 12 carbons in length (e.g., greater than or equal to 13, 14, 15, 16, 17, 18, 19, or 20 carbons in length, or ranges including and/or spanning the aforementioned values). In some aspects, the co-emulsifier component is a single lipid. In some aspects, the co-emulsifier component is highly pure. In some aspects, the co-emulsifier component has a purity by weight % of equal to or greater than about: 90%, 95%, 97%, 98%, 99%, 100%, or ranges including and/or spanning the aforementioned values. In some aspects, the co-emulsifier component is present in the nanoparticle composition at dry weight % of equal to or greater than about: 10%, 20%, 30%, 35%, 40%, 45%, 50%, or ranges including and/or spanning the aforementioned values.

In some aspects, the nanoparticle composition comprises one or more sterols. In certain aspects, the nanoparticle composition does not comprise a sterol. In some aspects, the one or more sterols comprises one or more cholesterols, ergosterols, hopanoids, hydroxysteroids, phytosterols (e.g., vegapure), ecdysteroids, and/or steroids. In some aspects the sterol comprises a cholesterol. In some aspects, the sterol component is a single sterol. In some aspects, the sterol component is cholesterol. In some aspects, the cholesterol (or other sterol) is highly pure. In some aspects, the one or more sterol(s) (e.g., cholesterol, and/or other sterols), collectively or individually, are present in the aqueous composition at a concentration of less than or equal to about: 50 mg/mL, 40 mg/mL, 20 mg/mL, 10 mg/mL, 5 mg/mL, or ranges including and/or spanning the aforementioned values. In some aspects, the one or more sterol(s) are present in the composition at a dry wt. % of equal to or less than about: 0.25%, 0.5%, 1%, 5%, 7.5%, 10%, 15%, 20%, 25%, or ranges including and/or spanning the aforementioned values. In some aspects, the one or more sterol(s) (collectively or individually) are present in the composition at a wet wt. % of equal to or less than about: 0.1%, 0.25%, 0.5%, 1%, 2%, 3%, 4%, 5%, 7.5%, 10%, or ranges including and/or spanning the aforementioned values. In some aspects, the cholesterol used in the composition comprises cholesterol from one or more of sheep's wool, synthetic cholesterol, or semisynthetic cholesterol from plant origin. In some aspects, the sterol has a purity of greater than or equal to about: 92.5%, 95%, 96%, 98%, 99%, 99.9%, 100.0%, or ranges including and/or spanning the aforementioned values. In some aspects, the sterol has a total % impurity content by weight of less than or equal to about: 8.5%, 5%, 4%, 3.7%, 2%, 1%, 0%, or ranges including and/or spanning the aforementioned values. In some aspects, the sterol is not cholesterol.

In several aspects, the nanoparticle composition comprises a preservative. In certain aspects, the nanoparticle composition does not comprise a preservative. In several aspects, the preservative includes one or more benzoates (such as sodium benzoate or potassium benzoate), nitrites (such as sodium nitrite), sulfites (such as sulfur dioxide, sodium or potassium sulphite, bisulphite or metabisulphite), sorbates (such as sodium sorbate, potassium sorbate), ethylenediaminetetraacetic acid (EDTA) (and/or the disodium salt thereof), polyphosphates, organic acids (e.g., citric, succinic, malic, tartaric, benzoic, lactic and propionic acids), and/or antioxidants (e.g., vitamins such as vitamin E and/or vitamin C, butylated hydroxytoluene). In several aspects, sorbates and benzoates may be used in acidic pH formulations. In several aspects, the one or more preservatives (collectively or individually) are present in the composition at a dry wt. % of equal to or at less than about: 0.01%, 0.1%, 0.25%, 0.5%, 1%, 5%, 7.5%, 10%, 15%, 20%, 25%, or ranges including and/or spanning the aforementioned values. In several aspects, the one or more preservatives (collectively or individually) are present in the composition at a wet wt. % of equal to or less than about: 0.001%, 0.01%, 0.025%, 0.05%, 0.1%, 0.5%, 0.75%, 1.0%, 1.5%, 2.0%, 2.5%, 5%, or ranges including and/or spanning the aforementioned values. In several aspects, the one or more preservatives (collectively or individually) are present in the composition at a wet w/v of equal to or less than about: 0 mg/mL, 0.001 mg/mL, 0.1 mg/mL, 0.5 mg/mL, 1.0 mg/mL, 2.5 mg/mL, 4 mg/mL, 5 mg/mL, or ranges including and/or spanning the aforementioned values. In several aspects, as disclosed elsewhere herein, the composition is aqueous, while in others it has been dried into a powder. For instance, as disclosed elsewhere herein, in several aspects, the composition is aqueous (wet), while in others it has been dried into a powder (dry). In several aspects, the preservatives inhibit or prevent growth of mold, bacteria, and fungus.

In some aspects, the nanoparticle composition comprises a metal. The metal may be zinc. In certain aspects, the nanoparticle composition does not comprise a metal. In several aspects, the one or more metals (collectively or individually) are present in the composition at a dry wt. % of equal to or at less than about: 0.01%, 0.1%, 0.25%, 0.5%, 1%, 5%, 7.5%, 10%, 15%, 20%, 25%, or ranges including and/or spanning the aforementioned values. In several aspects, the one or more metals (collectively or individually) are present in the composition at a wet wt. % of equal to or less than about: 0.001%, 0.01%, 0.025%, 0.05%, 0.1%, 0.5%, 0.75%, 1.0%, 1.5%, 2.0%, 2.5%, 5%, or ranges including and/or spanning the aforementioned values. In several aspects, the one or more metals (collectively or individually) are present in the composition at a wet w/v of equal to or less than about: 0 mg/mL, 0.001 mg/mL, 0.1 mg/mL, 0.5 mg/mL, 1.0 mg/mL, 2.5 mg/mL, 4 mg/mL, 5 mg/mL, or ranges including and/or spanning the aforementioned values. The metals may be combined with metalloligands to generate a nanoscale coordination polymer. Nanoscale coordination polymers may comprise metal-connecting points and organic bridging ligands. The nanoscale coordination polymers (NCP) may self-assemble into nanoparticles.

In several aspects, as disclosed elsewhere herein, the nanoparticle composition provides particles in the nano-measurement range. In several aspects, the nanoparticle is spherical or substantially spherical. In several aspects, a solid lipid nanoparticle possesses a solid lipid core matrix that can solubilize lipophilic molecules. In several aspects, the lipid core is stabilized by surfactants and/or emulsifiers as disclosed elsewhere herein, while in other aspects, surfactants are absent. In several aspects, the size of the particle is measured as a mean diameter. In several aspects, the size of the particle is measured by dynamic light scattering. In several aspects, the size of the particle is measured using a zeta-sizer. In several aspects, the size of the particle can be measured using Scanning Electron Microscopy (SEM). In several aspects, the size of the particle is measured using a cyrogenic SEM (cryo-SEM). Where the size of a nanoparticle is disclosed elsewhere herein, any one or more of these instruments or methods may be used to measure such sizes.

In several aspects, the nanoparticle composition comprises nanoparticles having an average size of less than or equal to about: 10 nm, 25 nm, 40 nm, 50 nm, 100 nm, 250 nm, 500 nm, 1000 nm, or ranges including and/or spanning the aforementioned values. In several aspects, the composition comprises nanoparticles having an average size of between about 50 nm and 150 nm or between about 50 and about 250 nm. In several aspects, the size distribution of the nanoparticles for at least 50%, 75%, 80%, 90% (or ranges including and/or spanning the aforementioned percentages) of the particles present is equal to or less than about: 20 nm, 40 nm, 60 nm, 80 nm, 100 nm, 110 nm, 120 nm, 130 nm, 140 nm, 160 nm, 180 nm, 200 nm, 300 nm, 400 nm, 500 nm, or ranges including and/or spanning the aforementioned nm values. In several aspects, the composition comprises nanoparticles having an average size of less than or equal to about: 10 nm, 50 nm, 100 nm, 250 nm, 500 nm, 1000 nm, or ranges including and/or spanning the aforementioned values. In several aspects, the size distribution of the nanoparticles for at least 90% of the particles present is equal to or less than about: 20 nm, 40 nm, 60 nm, 80 nm, 100 nm, 110 nm, 120 nm, 130 nm, 140 nm, 160 nm, 180 nm, 200 nm, 300 nm, 400 nm, 500 nm, or ranges including and/or spanning the aforementioned nm values. In several aspects, the size distribution of the nanoparticles for at least 90% of the particles present is equal to or less than about: 100 nm, 110 nm, 120 nm, 130 nm, 140 nm, 160 nm, 180 nm, 200 nm, or ranges including and/or spanning the aforementioned nm values. In several aspects, the D90 of the particles present is equal to or less than about: 80 nm, 100 nm, 110 nm, 120 nm, 130 nm, 140 nm, 160 nm, 180 nm, 200 nm, 300 nm, 400 nm, 500 nm, or ranges including and/or spanning the aforementioned values. In several aspects, the size of the nanoparticle is the diameter of the nanoparticle as measured using any of the techniques as disclosed elsewhere herein. For instance, in some aspects, the size of the nanoparticle is the measured using dynamic light scattering. In several aspects, the size of the nanoparticle is the measured using a zeta sizer. In several aspects, consistency in size over time, or within a sample, allows predictable stability for the active agent encapsulated therein.

In several aspects, over 50%, 75%, 95% (or ranges spanning and or including the aforementioned values) of the nanoparticles prepared by the methods disclosed herein have a particle size of between about 20 to about 500 nm (as measured by zeta sizing (e.g., refractive index). In several aspects, over 50%, 75%, 95% (or ranges spanning and or including the aforementioned values) of the nanoparticles prepared by the methods disclosed herein have a particle size of between about 50 nm to about 200 nm (as measured by zeta sizing (e.g., refractive index). In several aspects, over 50%, 75%, 95% (or ranges spanning and or including the aforementioned values) of the nanoparticles prepared by the methods disclosed herein have a particle size of between about 90 nm to about 150 nm (as measured by zeta sizing (e.g., refractive index). In several aspects, this consistency in size allows predictable delivery to subjects. In several aspects, the D90 particle size measurement varies between 150 and 500 nm.

In several aspects, the average size of the nanoparticles of a composition as disclosed herein is substantially constant and/or does not change significantly over time (e.g., it is a stable nanoparticle). In several aspects, after formulation and storage for a period of at least about 1 month (30 days), about 3 months (90 days), or about 6 months (180 days) (e.g., at ambient conditions, at 25° C. with 60% relative humidity, or under the other testing conditions disclosed elsewhere herein), the average size of nanoparticles comprising the composition changes less than or equal to about: 1%, 5%, 10%, 20%, or ranges including and/or spanning the aforementioned values.

In several aspects, the polydispersity index (PDI) of the nanoparticles of a composition as disclosed herein is less than or equal to about: 0.05, 0.10, 0.15, 0.20, 0.25, 0.30, 0.40, 0.50, 0.60, 0.70, 0.80, or ranges including and/or spanning the aforementioned values. In several aspects, the size distribution of the nanoparticles is highly monodisperse with a polydispersity index of less than or equal to about: 0.05, 0.10, 0.15, 0.20, 0.25, or ranges including and/or spanning the aforementioned values.

In several aspects, the zeta potential of the nanoparticles of a composition as disclosed herein is less than or equal to about: 1 mV, 3 mV, 4 mV, 5 mV, 6 mV, 7 mV, 8 mV, 10 mV, 20 mV, or ranges including and/or spanning the aforementioned values. In several aspects, the zeta potential of the nanoparticles is greater than or equal to about: −3 mV, −1 mV, 0 mV, 1 mV, 3 mV, 4 mV, 5 mV, 6 mV, 7 mV, 8 mV, 4 mV, 10 mV, 20 mV, or ranges including and/or spanning the aforementioned values. In several aspects, the zeta potential and/or diameter of the particles (e.g., measured using dynamic light scattering) is acquired using a zetasizer (e.g., a Malvern ZS90 or similar instrument).

In several aspects, as disclosed elsewhere herein, the nanoparticle composition is an oil-in-water emulsion, water-in-oil emulsion, water-in-oil-in-water emulsion, oil-in-water-in-oil emulsion, liposome, solid lipid particles formulation, etc. For brevity, these may just be referred to as the composition. In several aspects, the nanoparticle composition can be processed to comprises one or more of solid lipid nanoparticles, liposomes (and variants including multi-lamellar, double liposome preparations, etc.), niosomes, ethosomes, electrostatic particulates, microemulsions, nanoemulsions, microsuspensions, nanosuspensions, or combinations thereof. In several aspects, polymeric nanoparticles may be formed. In several aspects, cyclodextrin is added.

In several aspects, a solid lipid nanoparticle compositions comprises a lipid core matrix. In several aspects, the lipid core matrix is solid. In several aspects, the solid lipid comprises one or more ingredients as disclosed elsewhere herein. In several aspects, the core of the solid lipid comprises one or more lipids, surfactants, active ingredients, etc. In several aspects, the surfactant acts as an emulsifier. In several aspects, emulsifiers can be used to stabilize the lipid dispersion (with respect to charge and molecular weight). In several aspects, the core ingredients (e.g., the components of the core) are present in the composition (collectively or individually) at a dry wt. % of equal to or less than about: 0.5%, 1.0%, 2.5%, 5%, 10%, 15%, 20%, 30%, 40%, 50%, 60%, 80% or ranges including and/or spanning the aforementioned values. In several aspects, the core ingredients and/or the emulsifiers (collectively or individually) are present in the composition at a wet wt. % of equal to or less than about: 0.5%, 1.0% 2.5%, 5%, 7.5%, 10%, 12.5%, 15%, 20%, 30%, 40%, 60% or ranges including and/or spanning the aforementioned values.

In several aspects, the nanoparticle composition (e.g., when in water or dried) comprises multilamellar nanoparticle vesicles, unilamellar nanoparticle vesicles, multivesicular nanoparticles, emulsion particles, irregular particles with lamellar structures and bridges, partial emulsion particles, combined lamellar and emulsion particles, and/or combinations thereof. In certain aspects, the nanoparticle compositions do not comprise multilamellar nanoparticle vesicles, unilamellar nanoparticle vesicles, multivesicular nanoparticles, emulsion particles, irregular particles with lamellar structures and bridges, partial emulsion particles, combined lamellar and emulsion particles, and/or combinations thereof. In several aspects, the composition is characterized by having multiple types of particles (e.g., lamellar, emulsion, irregular, etc.). In other aspects, a majority of the particles present are emulsion particles. In several aspects, a majority of the particles present are lamellar (multilamellar and/or unilamellar). In other aspects, a majority of the particles present are irregular particles. In still other aspects, a minority of the particles present are emulsion particles. In several aspects, a minority of the particles present are lamellar (multilamellar and/or unilamellar). In other aspects, a minority of the particles present are irregular particles.

In several aspects, multilamellar nanoparticles comprise equal to or at least about 5%, 8%, 9%, 10%, 15%, 25%, 50%, 75%, 85%, 95%, or 100% (or ranges spanning and/or including the aforementioned values) of the particles present in the composition (e.g., the aqueous composition). For example, in some aspects, between about 5% and about 10% of the particles present are multilamellar. In several aspects, unilamellar nanoparticles comprise equal to or at least about 5%, 8%, 9%, 10%, 15%, 20%, 25%, 50%, 75%, 85%, 95%, or 100% (or ranges spanning and/or including the aforementioned values) of the particles present in the composition (e.g., the aqueous composition). For example, in some aspects, between about 10% and about 15% of the particles present are unilamellar.

In several aspects, emulsion particles comprise equal to or at least about 5%, 8%, 9%, 10%, 15%, 25%, 50%, 60%, 65%, 70%, 75%, 85%, 95%, or 100% (or ranges spanning and/or including the aforementioned values) of the particles present in the composition (e.g., the aqueous composition). For example, in some aspects, between about 60% to about 75% of the particles present are emulsion particles.

In several aspects, micelle particles comprise equal to or at least about 5%, 8%, 9%, 10%, 15%, 25%, 50%, 60%, 65%, 70%, 75%, 85%, 95%, or 100% (or ranges spanning and/or including the aforementioned values) of the particles present in the composition (e.g., the aqueous composition).

In several aspects, liposomes comprise equal to or at least about 5%, 8%, 9%, 10%, 15%, 25%, 50%, 60%, 65%, 70%, 75%, 85%, 95%, or 100% (or ranges spanning and/or including the aforementioned values) of the particles present in the composition (e.g., the aqueous composition).

In several aspects, irregular particles (including particles with lamellar structures and/or bridges) comprise equal to or at least about 1%, 2%, 3%, 4%, 5%, 6%, 7%, 8%, 9%, 10%, 15%, 25%, 50%, 75%, 85%, 95%, or 100% (or ranges spanning and/or including the aforementioned values) of the particles present in the composition (e.g., the aqueous composition). For example, in some aspects, between about 1% to about 5% of the particles present are irregular particles.

In several aspects, combined lamellar and emulsion particles comprise equal to or at least about 5%, 6%, 7%, 8%, 9%, 10%, 15%, 25%, 50%, 75%, 85%, 95%, or 100% (or ranges spanning and/or including the aforementioned values) of the particles present in the composition (e.g., the aqueous composition).

In several aspects, mixed-micelle particles comprise equal to or at least about 5%, 6%, 7%, 8%, 9%, 10%, 15%, 25%, 50%, 75%, 85%, 95%, or 100% (or ranges spanning and/or including the aforementioned values) of the particles present in the composition (e.g., the aqueous composition).

The nanoparticle compositions can comprise, but are not limited to, combinations of multilamellar nanoparticles, unilamellar nanoparticles, emulsion nanoparticles, micelle nanoparticles, irregular particles, and/or liposomes.

The percentages and/or concentrations of particles present in the composition may be purposefully modified. In some aspects, the percentage and/or concentration of the particles present in the composition are tailored to the active compound and/or the liquid comprising the particles. Such tailoring may lead to more homogenization and/or dispersion in the liquid. The tailoring may stabilize dispersion in the liquid.

In several aspects, the formulations and/or compositions disclosed herein are stable during sterilization. In several aspects, the sterilization may include one or more of ozonation, UV treatment, and/or heat treatment. In several aspects, the particle size and/or PDI after sterilization (e.g., exposure to techniques that allow sterilization of the composition) varies by less than or equal to about: 1%, 5%, 10%, 20%, 30%, or ranges including and/or spanning the aforementioned values. In several aspects, the therapeutic agent concentration after sterilization (e.g., exposure to techniques that allow sterilization of the composition) varies (e.g., drops) by less than or equal to about: 1%, 5%, 10%, 15%, or ranges including and/or spanning the aforementioned values.

In several aspects, the nanoparticle compositions (including after stabilization) disclosed herein have a shelf life of equal to or greater than 1 month, 3 months, 6 months, 12 months, 14 months, 16 months, 18 months, 19 months, or ranges including and/or spanning the aforementioned values. The shelf-life can be determined as the period of time in which there is 95% confidence that at least 50% of the response (active agent(s) concentration or particle size) is within the specification limit. This refers to a 95% confidence interval and when linear regression predicts that at least 50% of the response is within the set specification limit.

### III. Cancer Therapy

Some methods of the disclosure relate to reducing the risk of a secondary tumor in a subject diagnosed with a primary cancer. The term “primary cancer,” as used herein, refers to the original, or first, tumor or site (organ or tissue) where cancer began in the body. The term “cancer,” as used herein, may be used to describe a solid tumor, metastatic cancer, or non-metastatic cancer. The cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus.

The disclosed methods may employed to reduce the risk of a secondary tumor in a subject diagnosed with a primary cancer. The methods disclosed herein can be administered in conjunction with or subsequent to a primary cancer therapy to treat a primary cancer. The cancer therapy may comprise a local cancer therapy. The cancer therapy may exclude a systemic cancer therapy. The cancer therapy may exclude a local therapy. The cancer therapy may comprise a local cancer therapy without the administration of a system cancer therapy. The cancer therapy may comprise administering a small molecule inducer of trained immunity of the present disclosure. The cancer therapy may comprise a radiotherapy. The cancer therapy may comprise a chemotherapy. The cancer therapy may comprise an immunotherapy, which may be a checkpoint inhibitor therapy. Any of these cancer therapies may also be excluded. Combinations of these therapies may also be administered.

The cancer may be a Stage I cancer. The cancer may be a Stage II cancer. The cancer may be a Stage III cancer. The cancer may be a Stage IV cancer. The primary cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

Methods may involve the determination, administration, or selection of an appropriate cancer “management regimen” and predicting the outcome of the same. As used herein the phrase “management regimen” refers to a management plan that specifies the type of examination, screening, diagnosis, surveillance, care, and treatment (such as dosage, schedule and/or duration of a treatment) provided to a subject in need thereof (e.g., a subject diagnosed with cancer).

The disclosed methods may employed to reduce the risk of a secondary tumor in a subject diagnosed with a primary cancer that is treated with radiotherapy. Radiotherapy, such as ionizing radiation, may be administered to a subject to treat a primary cancer. As used herein, “ionizing radiation” means radiation comprising particles or photons that have sufficient energy or can produce sufficient energy via nuclear interactions to produce ionization (gain or loss of electrons). A preferred non-limiting example of ionizing radiation is an x-radiation. Means for delivering x-radiation to a target tissue or cell are well known in the art.

The radiotherapy can comprise external radiotherapy, internal radiotherapy, radioimmunotherapy, or intraoperative radiation therapy (IORT). The external radiotherapy may comprise three-dimensional conformal radiation therapy (3D-CRT), intensity modulated radiation therapy (IMRT), proton beam therapy, image-guided radiation therapy (IGRT), or stereotactic radiation therapy. The internal radiotherapy may comprise interstitial brachytherapy, intracavitary brachytherapy, or intraluminal radiation therapy. The radiotherapy may be administered to a primary tumor.

The amount of ionizing radiation is greater than 20 Gy and may be administered in one dose. The amount of ionizing radiation may be 18 Gy and is administered in three doses. The amount of ionizing radiation may be at least, at most, or exactly 0.5, 1, 2, 4, 6, 8, 10, 15, 16, 17, 18, 19, 20, 21, 22, 23, 24, 25, 26, 27, 18, 19, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 Gy (or any derivable range therein). The ionizing radiation may be administered in at least, at most, or exactly 1, 2, 3, 4, 5, 6, 7, 8, 9, or 10 does (or any derivable range therein). When more than one dose is administered, the does may be about 1, 4, 8, 12, or 24 hours or 1, 2, 3, 4, 5, 6, 7, or 8 days or 1, 2, 3, 4, 5, 6, 7, 8, 9, 10, 12, 14, or 16 weeks apart, or any derivable range therein.

The amount of radiotherapy administered to a subject may be presented as a total dose of radiotherapy, which is then administered in fractionated doses. For example, the total dose may be 50 Gy administered in 10 fractionated doses of 5 Gy each. The total dose may be 50-90 Gy, administered in 20-60 fractionated doses of 2-3 Gy each. The total dose of radiation may be at least, at most, or about 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, or 150 Gy (or any derivable range therein). The total dose may be administered in fractionated doses of at least, at most, or exactly 1, 2, 3, 4, 5, 6, 7, 8, 9, 10, 12, 14, 15, 20, 25, 30, 35, 40, 45, or 50 Gy (or any derivable range therein). At least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fractionated doses may be administered (or any derivable range therein). At least, at most, or exactly 1, 2, 3, 4, 5, 6, 7, 8, 9, 10, 11, or 12 (or any derivable range therein) fractionated doses may be administered per day. At least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 (or any derivable range therein) fractionated doses may be administered per week.

The disclosed methods may employed to reduce the risk of a secondary tumor in a subject diagnosed with a primary cancer that is treated with a cancer immunotherapy. Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumor-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines.

The disclosed methods may employed to reduce the risk of a secondary tumor in a subject diagnosed with a primary cancer that is treated with an immune checkpoint inhibitor. As disclosed herein, “checkpoint inhibitor therapy” (also “immune checkpoint blockade therapy”, “immune checkpoint therapy”, “ICT,” “checkpoint blockade immunotherapy,” or “CBI”), refers to cancer therapy comprising providing one or more immune checkpoint inhibitors to a subject suffering from or suspected of having cancer.

The immunotherapy may comprise an activator of a co-stimulatory molecule. The activator may comprise an agonist of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Activators include agonistic antibodies, polypeptides, compounds, and nucleic acids.

The disclosed methods may employed to reduce the risk of a secondary tumor in a subject diagnosed with a primary cancer that is treated with dendritic cells. Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7, TLR8 or CD40 have been used as antibody targets.

The disclosed methods may employed to reduce the risk of a secondary tumor in a subject diagnosed with a primary cancer that is treated with chimeric T cell receptors. Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell. The receptors are called chimeric because they are fused of parts from different sources. CAR-T cell therapy refers to a treatment that uses such transformed cells for cancer therapy.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signaling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted. Example CAR-T therapies include Tisagenlecleucel (Kymriah) and Axicabtagene ciloleucel (Yescarta).

The disclosed methods may employed to reduce the risk of a secondary tumor in a subject diagnosed with a primary cancer that is treated with cytokines. Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins. Interferons are produced by the immune system. They are usually involved in anti-viral response, but also have use for cancer. They fall in three groups: type I (IFNα and IFNβ), type II (IFNγ) and type III (IFNλ). Interleukins have an array of immune system effects. IL-2 is an example interleukin cytokine therapy.

The disclosed methods may employed to reduce the risk of a secondary tumor in a subject diagnosed with a primary cancer that is treated with adoptive T cell therapy. Adoptive T cell therapy is a form of passive immunization by the transfusion of T-cells (adoptive cell transfer). They are found in blood and tissue and usually activate when they find foreign pathogens. Specifically they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumor death.

Multiple ways of producing and obtaining tumor targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

It is contemplated that a cancer treatment may exclude any of the cancer treatments described herein. Methods and compositions of the disclosure include patients that have been previously treated for a therapy described herein, are currently being treated for a therapy described herein, or have not been treated for a therapy described herein. The patient may be one that has been determined to be resistant to a therapy described herein. The patient may be one that has been determined to be sensitive to a therapy described herein.

The disclosed methods may employed to reduce the risk of a secondary tumor in a subject diagnosed with a primary cancer that is treated with a chemotherapeutic agent, i.e., a chemotherapy. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin), substituted ureas (e.g., hydroxyurea), methylhydiazine derivatives (e.g., procarbazine), and adreocortical suppressants (e.g., taxol and mitotane). Cisplatin may be used as a particularly suitable chemotherapeutic agent.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection. Cisplatin can be used alone or in combination with other agents, with efficacious doses used in clinical applications including about 15 mg/m² to about 20 mg/m² for 5 days every three weeks for a total of three courses being contemplated.

Other suitable chemotherapeutic agents include antimicrotubule agents, e.g., Paclitaxel (“Taxol”) and doxorubicin hydrochloride (“doxorubicin”). The combination of an Egr-1 promoter/TNFα construct delivered via an adenoviral vector and doxorubicin was determined to be effective in overcoming resistance to chemotherapy and/or TNF-α, which suggests that combination treatment with the construct and doxorubicin overcomes resistance to both doxorubicin and TNF-α.

Nitrogen mustards are another suitable chemotherapeutic agent useful in the methods of the disclosure. A nitrogen mustard may include, but is not limited to, mechlorethamine (HN2), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred. The drug also sometimes is administered intramuscularly, by infiltration or into body cavities.

Additional suitable chemotherapeutic agents include pyrimidine analogs, such as cytarabine (cytosine arabinoside), 5-fluorouracil (fluouracil; 5-FU) and floxuridine (fluorode-oxyuridine; FudR). 5-FU may be administered to a subject in a dosage of anywhere between about 7.5 to about 1000 mg/m2. Further, 5-FU dosing schedules may be for a variety of time periods, for example up to six weeks, or as determined by one of ordinary skill in the art to which this disclosure pertains. The amount of the chemotherapeutic agent delivered to the patient to treat a primary cancer may be variable. The chemotherapeutic agent may be administered in an amount effective to cause arrest or regression of the cancer in a host, when the chemotherapy is administered with the construct. The chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

The disclosed methods may employed to reduce the risk of a secondary tumor in a subject diagnosed with a primary cancer that is treated with a hormone therapy. In particular aspects, a prostate cancer therapy comprises hormone therapy. Various hormone therapies are known in the art and contemplated herein. Examples of hormone therapies include, but are not limited to, luteinizing hormone-releasing hormone (LHRH) analogs, LHRH antagonists, androgen receptor antagonists, and androgen synthesis inhibitors.

### IV. BCL Proteins

The Bcl-2 proteins are a family of structurally related proteins that serve as central regulators of intrinsic programmed cell death. Bcl-2 family proteins are classified as either antiapoptotic or proapoptotic proteins. Antiapoptotic members (Bcl-xL, Bcl-2, Bcl-w and Mcl-1) contain four Bcl-2 homology (BH1-4) domains. They localize to the cytosol and to the mitochondrial and endoplasmic reticulum membrane. Proapoptotic members are further classified as multidomain proteins that contain three BH domains: BH1-3 (Bak and Bax) and the “BH3-only” proteins (Bid, Bad, Bim, Puma, Noxa BIK, BMF, HKR/DP5) of unknown structure (except for Bid, which adopts the overall structure of the Bcl-2 family proteins). The members of the Bcl-2 family share one or more of the four characteristic domains of homology entitled the Bcl-2 homology (BH) domains (named BH1, BH2, BH3 and BH4) (see figure). The BH domains are known to be crucial for function, as deletion of these domains via molecular cloning affects survival/apoptosis rates. The anti-apoptotic Bcl-2 proteins, such as Bcl-2 and Bcl-xL, conserve all four BH domains. The BH domains also serve to subdivide the pro-apoptotic Bcl-2 proteins into those with several BH domains (e.g. Bax and Bak) or those proteins that have only the BH3 domain (e.g. Bim Bid and BAD).

Because they are thought to induce cell death by inhibiting the antiapoptotic family members, BH3-only proteins are divided into two groups: the “inactivators” or “indirect activators” (e.g., Bad, Noxa) and the “direct activators” (e.g., tBid and, possibly, also Bim and Puma), which bind transiently and activate the multidomain proteins Bax and Bak. The balance between antiapoptotic and proapoptotic Bcl-2 family members has long been thought to determine the functional integrity of the mitochondrial outer membrane and commitment to cell death (see later). When the abundance of Bax, Bim, Bad, and Bid exceeds that of antiapoptotic Bcl-2 family members, it promotes the release of cytochrome c and other apoptogenic factors from the mitochondria, which initiate apoptosis. Cell death stimuli can shift this balance in favor of apoptosis by regulation of Bcl-2 protein abundance, post-translational modification, and activity.

Antiapoptotic Bcl-2-family proteins are known for their ability to prolong survival of growth factor-dependent cells when deprived of their obligate growth factors. Bcl-2 and Bcl-XL suppress autophagy by binding the protein Beclin (ATG7), an essential component of the mammalian autophagy system that marks autophagic vesicles for fusion with lysosomes for digestion and recycling of components. The antiautophagic function of Bcl-2 has been dissociated from mitochondrial location, and instead is manifested from the endoplasmic reticulum (ER). In this regard, Bcl-2-family proteins have regulatory effects on several proteins and processes in the ER, including those influencing the unfolded protein response (UPR). The UPR is an evolutionarily conserved adaptive mechanism that detects accumulation of unfolded proteins in the lumen of the ER, causing induction of chaperones and transporters that strive to restore homeostasis, at least in part, by retrograde transport of unfolded proteins into the cytosol for ubiquitination and proteasome-mediated destruction.

### V. General Considerations

Trained immunity is an innate immune memory that induces enhanced inflammatory and antimicrobial properties in innate immune cells. Trained immunity can be initiated or triggered by particular molecules,¹ and is associated with metabolic and epigenetic rewiring of innate immune cells that improves response to subsequent inflammatory challenges.² In some aspects, trained immunity is characterized by an increase in the concentration of at least one cytokine. In some aspects, the increase in concentration of at least one cytokine is an increase of at least 1.6-fold greater than that which would have occurred if the cell or subject had not been contacted with the small molecule inducer of trained immunity. In some aspects, the at least one cytokine is tumor necrosis factor-alpha (TNF-α) or interleukin-6 (IL-6). In some aspects, the degree to which trained immunity is induced by a small molecule inducer of trained immunity is determined by a change in TNF-α and/or IL-6 levels. TNF-α and/or IL-6 levels can be measured after administration of a small molecule inducer of trained immunity. In some aspects, TNF-α and/or IL-6 levels are measured on the day after administration of a small molecule inducer of trained immunity. In some aspects, TNF-α and/or IL-6 levels are measured on the first, second, third, fourth, fifth, sixth, seventh, eighth, ninth, tenth, eleventh, twelfth, thirteenth, fourteenth, or fifteenth day after administration of a small molecule inducer of trained immunity. In some aspects, TNF-α and/or IL-6 levels are measured on the seventh day after administration of a small molecule inducer of trained immunity. In some aspects, trained immunity is associated with epigenetic reprogramming which leads to a stronger response to an immunological challenge. In some aspects, epigenetic reprogramming is characterized by a change in chromatin accessibility, the degree of DNA methylation, and/or histone modification. In some aspects, trained immunity is associated with an amplified response to an ensuing immunological challenge. In some aspects, the immunological challenge is an infectious agent that consists of or comprises a virus, a bacterium, a protozoan, a prion, a viroid, and a fungus. In some aspects, the immunological challenge is a secondary metastasis and the subject is a subject previously treated for a primary cancer. In some aspects, trained immunity is associated with a change in a metabolic process. In some aspects, the degree to which trained immunity is induced by an administered compound is measured by an assay for transposase-accessible chromatin with high-throughput sequencing (ATAC-Seq).

Initially, pathogen-derived stimuli like BCG and β-glucan were used as model inducers of trained immunity.³⁻⁵ Both of these model training inducers act through pattern recognition receptors, NOD2 and Dectin-1, respectively.^{6,7} Previous work also linked the role of certain endogenous metabolic or hormone molecules to trained immunity in the pathophysiology of atherosclerosis and diabetes.⁸⁻¹¹ Each of these inducers of trained immunity has been shown to reprogram metabolism and chromatin accessibility in conjunction with a primary immune response (as in BCG) or hyperinflammatory disease state.

According to one aspect of the presently disclosed subject matter, a small molecule, A1155463 (i.e., 2-(8-(benzo[d]thiazol-2-ylcarbamoyl)-3,4-dihydroisoquinolin-2(1H)-yl)-5-(3-(4-(3-(dimethyl-amino) prop-1-yn-1-yl)-2-fluorophenoxy)propyl)thiozole-4-carboxylic acid), that is neither pathogen-derived nor disease-associated, was identified as an inducer of trained immunity. According to a further aspect of the presently disclosed subject matter, additional small molecule inducers of trained immunity were identified using a high-throughput screening approach. Such a screen can provide for the identification of common phenotypes associated with training and the identification of safe, effective training compounds with therapeutic potential.

High-throughput screening has been used to identify small molecule drugs that influence cellular metabolism and epigenetic regulation.¹²⁻¹⁵ This supported the hypothesis that a screen could successfully identify trained immunity inducers, which often modulate both metabolism and epigenetic pathways.^{1,2,16} Most of these previous screens, however, use assays that only require the cells to survive for hours to days, and they typically use immortalized cell lines.¹⁵⁻¹⁷ The presently disclosed assay lasts 7 days to provide sufficient time between induction of training and stimulation of the cells, and it requires primary mouse bone marrow-derived macrophages (BMDMs), adding additional challenges to the screen.” Furthermore, high-throughput screening usually targets single receptors rather than measuring a functional response, as is required to measure trained immunity.

To compensate for these challenges, several quality control tests were incorporated during the screening assay to ensure the accuracy and consistency of the results across replicates.

Accordingly, described in the examples below is a high-throughput screen for identifying new small molecule inducers of trained immunity. Over 2 dozen small molecules (about 1.5% of the small molecules tested, which included steroids, pesticides, adrenergic agonists, antineoplastics, pro-apoptotic compounds and non-steroidal anti-inflammatory drugs (NSAIDs)) were identified as capable of inducing trained immunity in vitro. See Table 1 in the Examples, below. Thus, treatment of macrophage cells with the identified molecules allowed them to generate an elevated pro-inflammatory response to a subsequent challenge. More particularly, small molecules were identified as inducers of trained immunity if they provided for at least a 1.6-fold increase in TNF-α concentration in BMDMs relative to untrained controls after challenge with a toll-like receptor (TLR) agonist, i.e., Pam3-CSK4, several days after exposure of the BMDMs to the small molecule. The in vitro training capacity of 7 hit compounds was confirmed in vivo. In addition, ATAC-seq on BMDMs trained with these hit compounds can be performed to identify transcription factors and chromatin accessibility induced by the identified small molecule inducers of immunity, e.g., as compared to training with β-glucan.

Several of the molecules identified herein are distinct from other known inducers of trained immunity in that they do not induce acute cytokine responses and are not derived from pathogenic sources. For instance, the particular small molecule inducers of trained immunity identified herein include A1155463 and the compounds identified via high-throughput screening described in Table 1 of the Examples. In some aspects, the compound consists of or comprises A1155463, flunisolide, hydrocortisone, halcinonide, budesonide, fluocinonide, fluocinolone acetonide, clobetasol, prednisolone, triamcinolone, nitrofurazone, benzeraside, nerol, hydroquinone, 5-fluoroindole-2-carboxylic acid, myricetin, fenoterol, and pharmaceutically acceptable salts thereof. In some aspects, the compound consists of or comprises flunisolide, hydrocortisone, halcinonide, budesonide, fluocinonide, fluocinolone acetonide, clobetasol propionate, prednisolone, triamcinolone, triamcinolone diacetate, nitrofurazone, hydrocortisone acetate, benseraside hydrochloride, nerol, hydroquinone, 5-fluoroindole-2-carboxylic acid, myricetin, and fenoterol hydrobromide. In some aspects, the compound consists of or comprises flunisolide, hydrocortisone, nerol, hydroquinone, 5-fluoroindole-2-carboxylic acid, myricetin, and fenoterol hydrobromide. Any one or more of the preceding compounds may be excluded in certain aspects. In some aspects, the compound is A1155463. In contrast to previous trained immunity inducers, in general, the presently identified inducers of trained immunity are not naturally occurring in the body, thus, they are not involved in pathogenesis of disease or homeostasis. They are easy to make and purify. They do not induce a primary immune response (e.g., initial administration of the compound does not activate the innate immune system or induce production of effector T or B cells).

The small molecule inducers of trained immunity as identified herein can be used as prophylactics for infectious diseases, to reduce the risk of secondary tumors in cancer patients previously treated for a primary cancer, and to enhance the effectiveness of vaccines. For example, in some aspects, the small molecule inducer of trained immunity can be administered prophylactically on a regular (e.g., monthly) basis in a population at increased risk of infectious disease or a secondary cancer. Although some of the presently disclosed small molecule inducers can also have anti-inflammatory properties, as disclosed herein, they can provide a hyperimmune response upon challenge (via an infectious agent or vaccine) at a time period of about one week or more (or one month or more) after they are administered to a subject. In some aspects, the small molecule inducer of trained immunity suitable for use according to the presently disclosed subject matter is was selected from the group comprising a glucocorticoid (e.g., flunisolide, hydrocortisone, halcinonide, budesonide, flunisolide, fluocinonide, fluocinolone acetonide, prednisolone, clobetasol, triamcinolone, nitrofurazone, or benserazide), A1155463, nerol, hydroquinone, 5-fluoroindole-2-carboxylic acid, myricetin, fenoterol or a pharmaceutically acceptable salt thereof (e.g., fenoterol hydrobromide, clobetasol propionate, triamcinolone diacetate, hydrocortisone acetate, or benserazide hydrochloride). Any one or more of the preceding compounds may be excluded in certain aspects.

### VI. Formulations

In some aspects, compositions comprising a small molecule inducer of trained immunity of the presently disclosed subject matter can comprise a pharmaceutically acceptable salt. Such salts include, but are not limited to, pharmaceutically acceptable acid addition salts, pharmaceutically acceptable base addition salts, pharmaceutically acceptable metal salts, ammonium and alkylated ammonium salts, and combinations thereof.

Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, paminobenzoic, glutamic, benzenesulfonic, p-toluenesulfonic acids, sulphates, nitrates, phosphates, perchlorates, borates, acetates, benzoates, hydroxynaphthoates, glycerophosphates, ketoglutarates and the like.

Base addition salts include but are not limited to, ethylenediamine, N-methyl-glucamine, lysine, arginine, ornithine, choline, N, N′-dibenzylethylenediamine, chloroprocaine, diethanolamine, procaine, N-benzylphenethylamine, diethylamine, piperazine, tris (hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e. g., lysine and arginine dicyclohexylamine and the like.

Examples of metal salts include lithium, sodium, potassium, magnesium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like.

The compositions of the presently disclosed subject matter comprise in some aspects, a composition that includes a pharmaceutically acceptable carrier. Any suitable pharmaceutical formulation can be used to prepare the compositions for administration to a subject. In some aspects, the composition and/or carriers can be pharmaceutically acceptable in humans. In some aspects, the carrier is non-toxic, biocompatible and is selected so as not to detrimentally affect the biological activity of the agent. The agents in some aspects of the disclosure may be formulated into preparations for local delivery (i.e. to a specific location of the body, such as or other tissue) or systemic delivery, in solid, semi-solid, gel, liquid or gaseous forms such as tablets, capsules, powders, granules, ointments, solutions, depositories, inhalants and injections allowing for oral, parenteral or surgical administration.

Suitable carriers for parenteral delivery via injectable, infusion or irrigation and topical delivery include distilled water, physiological phosphate-buffered saline, normal or lactated Ringer's solutions, dextrose solution, Hank's solution, or propanediol. In addition, sterile, fixed oils may be employed as a solvent or suspending medium. For this purpose any biocompatible oil may be employed including synthetic mono- or diglycerides. In addition, fatty acids such as oleic acid find use in the preparation of injectables. The carrier and agent may be compounded as a liquid, suspension, polymerizable or non-polymerizable gel, paste or salve.

The carrier may also comprise a delivery vehicle to sustain (i.e., extend, delay or regulate) the delivery of the agent(s) or to enhance the delivery, uptake, stability or pharmacokinetics of the therapeutic agent(s). Such a delivery vehicle may include, by way of non-limiting examples, microparticles, microspheres, nanospheres or nanoparticles composed of proteins, liposomes, carbohydrates, synthetic organic compounds, inorganic compounds, polymeric or copolymeric hydrogels and polymeric micelles.

In certain aspects, the actual dosage amount of a composition administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

Suitable formulations can include aqueous and non-aqueous sterile injection solutions that can contain anti-oxidants, buffers, bacteriostatics, bactericidal antibiotics, and solutes that render the formulation isotonic with the bodily fluids of the subject; and aqueous and non-aqueous sterile suspensions that can include suspending agents and thickening agents. The formulations can be presented in unit-dose or multi-dose containers, for example sealed ampoules and vials, and can be stored in a frozen or freeze-dried (lyophilized) condition requiring only the addition of sterile liquid carrier, for example water for injections, immediately prior to use. Some exemplary ingredients are sodium dodecyl sulfate (SDS), in one example in the range of 0.1 to 10 mg/ml, in another example about 2.0 mg/ml; and/or mannitol or another sugar, for example in the range of 10 to 100 mg/ml, in another example about 30 mg/ml; and/or phosphate-buffered saline (PBS). In some aspects, solutions of pharmaceutical compositions can be prepared in water suitably mixed with a surfactant, such as hydroxypropylcellulose. Dispersions also can be prepared in glycerol, liquid polyethylene glycols, mixtures thereof and in oils, for example. Under ordinary conditions of storage and use, these preparations contain a preservative to prevent the growth of microorganisms.

In certain aspects, the pharmaceutical compositions are administered in the form of injectable compositions either as liquid solutions or suspensions; solid forms suitable or solution in, or suspension in, liquid prior to injection may also be prepared. These preparations also may be emulsified. A typical composition for such purpose comprises a pharmaceutically acceptable carrier. For instance, the composition may contain 10 mg or less, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like.

Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, antifungal agents, anti-oxidants, chelating agents and inert gases. The pH and exact concentration of the various components the pharmaceutical composition are adjusted according to well-known parameters.

It should be understood that in addition to the ingredients particularly mentioned above, the formulations of this presently disclosed subject matter can include other agents conventional in the art having regard to the type of formulation in question. For example, sterile pyrogen-free aqueous and non-aqueous solutions can be used.

Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, antifungal agents, anti-oxidants, chelating agents and inert gases. The pH and exact concentration of the various components the pharmaceutical composition are adjusted according to well-known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders.

In further aspects, the pharmaceutical compositions may include classic pharmaceutical preparations. Administration of pharmaceutical compositions according to certain aspects may be via any common route so long as the target tissue is available via that route. This may include oral, nasal, buccal, rectal, vaginal or topical. Alternatively, administration may be by orthotopic, intradermal, subcutaneous, intramuscular, intraperitoneal or intravenous injection. Such compositions would normally be administered as pharmaceutically acceptable compositions that include physiologically acceptable carriers, buffers or other excipients. For treatment of conditions of the lungs, aerosol delivery can be used. Volume of the aerosol may be between about 0.01 ml and 0.5 ml, for example.

In some aspects, the small molecule inducer of trained immunity can be encapsulated in a nanoparticle delivery agent. For instance, the inducer molecule can be encapsulated in a nanoparticle comprising a biodegradable polymer, such as poly(glycolide-co-lactide) (PGLA). Use of a biodegradable polymer nanoparticle can provide for sustained, prolonged release of the encapsulated small molecule and/or, via retention of the nanoparticle in a particular site, targeted release at a particular site in the body, e.g., at or near the site of previously treated primary cancer.

The active compounds can be formulated for parenteral administration, e.g., formulated for injection via the intravenous, intramuscular, subcutaneous, or intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including, for example, aqueous propylene glycol; and sterile powders for the extemporaneous preparation of sterile injectable solutions or dispersions. In all cases the form must be sterile and should be fluid to the extent that it may be easily injected. It also should be stable under the conditions of manufacture and storage and must be preserved against the contaminating action of microorganisms, such as bacteria and fungi.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, example methods of preparation are vacuum-drying and freeze-drying techniques, which yield a powder of the active ingredient, plus any additional desired ingredient from a previously sterile-filtered solution thereof.

### VII. Subjects

The methods and compositions disclosed herein can be used on a sample either in vitro (for example, on isolated cells, cell cultures, or tissues) or in vivo in a subject (i.e. living organism, such as a patient). In some aspects, the subject is a human subject, although it is to be understood that the principles of the presently disclosed subject matter indicate that the presently disclosed subject matter is effective with respect to all vertebrate species, including mammals, which are intended to be included in the terms “subject” and “patient”. The subject may be a human subject. The subject may be a cow, pig, horse, cat, dog, mammal, non-human primate, rat, mouse, or rabbit. Moreover, a mammal is understood to include any mammalian species for which employing the compositions and methods disclosed herein is desirable, particularly agricultural and domestic mammalian species.

As such, the methods of the presently disclosed subject matter are particularly useful in warm-blooded vertebrates. Thus, the presently disclosed subject matter concerns mammals and birds. More particularly provided are methods and compositions for mammals such as humans, as well as those mammals of importance due to being endangered (such as Siberian tigers), of economic importance (animals raised on farms for consumption by humans), and/or of social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also provided is the treatment of birds, including the treatment of those kinds of birds that are endangered, kept in zoos or as pets (e.g., parrots), as well as fowl, and more particularly domesticated fowl, for example, poultry, such as turkeys, chickens, ducks, geese, guinea fowl, and the like, as they are also of economic importance to humans. Thus, also provided is the treatment of livestock including, but not limited to domesticated swine (pigs and hogs), ruminants, horses, poultry, and the like.

In some aspects, the subject is a subject with a weakened immune system, such as an elderly or juvenile subject or an immunocompromised subject. In some aspects, the subject is a subject at higher risk for infection with a pathogen, such as a subject traveling to a location experiencing an outbreak of a particular infection, a medical professional or other subject who works in a medical setting and has increased risk of exposure to infectious agents or infected individuals, or a subject living in a communal living situation (e.g., a college dormitory, apartment complex, a prison, an assisted living community, etc.).

### VIII. Administration

Suitable methods for administration of a composition of the presently disclosed subject matter include, but are not limited to intravenous injection, intramuscular injection, oral administration, subcutaneous administration, intraperitoneal injection, intracranial injection, rectal administration, ocular administration, and intranasal administration. In some aspects, the administration is systemic. In some aspects, the administration is at a site where contact with an infectious agent might occur, e.g., intranasal administration. In some aspects, a composition can be deposited at a site in need of treatment in any other manner, for example by spraying a composition within the pulmonary pathways. The particular mode of administering a composition of the presently disclosed subject matter depends on various factors, including the distribution and abundance of cells to be treated.

The therapy provided herein may comprise administration of a combination of therapeutic agents, such as a first a small molecule inducer of trained immunity and a second a small molecule inducer of trained immunity. The therapies may be administered in any suitable manner known in the art. For example, the first and second small molecule inducers of trained immunity may be administered sequentially (at different times) or concurrently (at the same time). In some aspects, the first and second small molecule inducers of trained immunity are administered in a separate composition. In some aspects, the first and second small molecule inducers of trained immunity are in the same composition.

In some aspects, the first small molecule inducer of trained immunity and the second small molecule inducer of trained immunity are administered substantially simultaneously. In some aspects, the first small molecule inducer of trained immunity and the second small molecule inducer of trained immunity are administered sequentially. In some aspects, the first small molecule inducer of trained immunity, the second small molecule inducer of trained immunity, and a third small molecule inducer of trained immunity are administered sequentially. In some aspects, the first small molecule inducer of trained immunity is administered before administering the second small molecule inducer of trained immunity. In some aspects, the first small molecule inducer of trained immunity is administered after administering the second small molecule inducer of trained immunity. In some aspects, the second small molecule inducer of trained immunity is administered after administering the first small molecule inducer of trained immunity. In some aspects, a first small molecule inducer of trained immunity is administered to a subject comprising a glucocorticoid, A1155463, nerol, hydroquinone, 5-fluoroindole-2-carboxylic acid, myricetin, fenoterol, or a pharmaceutically acceptable salt thereof; following this, a small molecule inducer of trained immunity is administered to a subject comprising a glucocorticoid, A1155463, nerol, hydroquinone, 5-fluoroindole-2-carboxylic acid, myricetin, fenoterol, or a pharmaceutically acceptable salt thereof. In certain aspects, the second small molecule inducer of trained immunity is administered at least 6, 7, 8, 9, 10, 11, 12, 13, 14, 15, 16, 17, 18, 19, 20, 21, 22, 23, 24, 28, 32, 36, 40, 44, 48, 72, or 96 hours (or any range or value derivable therein) after administering the first small molecule inducer of trained immunity. In some aspects, the second small molecule inducer of trained immunity is administered at least 1, 2, 3, 4, or 5 days following administration of the first small molecule inducer of trained immunity. In some aspects, the second small molecule inducer of trained immunity is administered the day after administration of the first small molecule inducer of trained immunity.

The therapeutic small molecule inducers of trained immunity of the disclosure may be administered by the same route of administration or by different routes of administration. In some aspects, the small molecule inducer of trained immunity is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

In certain instances, it will be desirable to have multiple administrations of the small molecule inducer of trained immunity, e.g., 2, 3, 4, 5, 6 or more administrations. The administrations can be at 1, 2, 3, 4, 5, 6, 7, 8, to 5, 6, 7, 8, 9, 10, 11, or 12 week intervals, including all ranges there between.

Administration of the compositions will typically be via any common route. This includes, but is not limited to oral, or intravenous administration. Alternatively, administration may be by orthotopic, intradermal, subcutaneous, intramuscular, intraperitoneal, or intranasal administration. Such compositions would normally be administered as pharmaceutically acceptable compositions that include physiologically acceptable carriers, buffers or other excipients.

Upon formulation, solutions will be administered in a manner compatible with the dosage formulation and in such amount as is therapeutically or prophylactically effective. The formulations are easily administered in a variety of dosage forms, such as the type of injectable solutions described above.

For delivery of compositions to pulmonary pathways, compositions of the presently disclosed subject matter can be formulated as an aerosol or coarse spray. Methods for preparation and administration of aerosol or spray formulations can be found, for example, in U.S. Pat. Nos. 5,858,784; 6,013,638; 6,022,737; and 6,136,295.

### IX. Doses

An effective dose of a composition of the presently disclosed subject matter is administered to a subject. An “effective amount” is an amount of the composition sufficient to produce detectable treatment. Actual dosage levels of constituents of the compositions of the presently disclosed subject matter can be varied so as to administer an amount of the composition that is effective to achieve the desired effect for a particular subject and/or target. The selected dosage level can depend upon the activity of the composition and the route of administration.

The treatments may include various “unit doses.” Unit dose is defined as containing a predetermined-quantity of the small molecule inducer of trained immunity. The quantity to be administered, and the particular route and formulation, is within the skill of determination of those in the clinical arts. A unit dose need not be administered as a single injection but may comprise continuous infusion over a set period of time. In some aspects, a unit dose comprises a single administrable dose.

The quantity to be administered, both according to number of treatments and unit dose, depends on the treatment effect desired. An effective dose is understood to refer to an amount necessary to achieve a particular effect. In the practice in certain aspects, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain aspects, the effective dose of the small molecule inducer of trained immunity is one which can provide a small molecule inducer of trained immunity blood level of about 1 μM to 150 μM. In another aspect, the effective dose of the small molecule inducer of trained immunity provides a blood level of about 4 μM to 100 μM.; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other aspects, the dose can provide the following blood level of the small molecule inducer of trained immunity that results from the small molecule inducer of trained immunity being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain aspects, the small molecule inducer of trained immunity that is administered to a subject is metabolized in the body to a metabolized small molecule inducer of trained immunity, in which case the blood levels may refer to the amount of the metabolized molecule. Alternatively, to the extent the small molecule inducer of trained immunity is not metabolized by a subject, the blood levels discussed herein may refer to the unmetabolized small molecule inducer of trained immunity.

Precise amounts of the amount of small molecule inducer of trained immunity that is administered also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

After review of the disclosure herein of the presently disclosed subject matter, one of ordinary skill in the art can tailor the dosages to an individual subject, taking into account the particular formulation, method of administration to be used with the composition, and nature of the target to be treated. Such adjustments or variations, as well as evaluation of when and how to make such adjustments or variations, are well known to those of ordinary skill in the art.

### EXAMPLES

The following Examples have been included to provide guidance to one of ordinary skill in the art for practicing representative aspects of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### General Materials and Methods

Animals: 6-week-old Female C57Bl/6J mice were obtained from Jackson Laboratories and acclimatized for 1 week before experimentation.

Chemicals: The screen was completed using Microsource Spectrum Library purchased through the Cellular Screening Center. A1155463, nerol, hydroquinone, 5-fluoroindole-2-carboxylic acid, myricetin, fenoterol, flunisolide, hydrocortisone, hydrocortisone acetate, halcinonide, budesonide, fluocinonide, fluocinolone acetonide, clobetasol propionate, prednisolone, triamcinolone, triamcinolone diacetate, nitrofurazone, and benserazide hydrochloride can be purchased from Sigma-Aldrich. A1155463 (CAS Number: 1235034-55-5, Sigma-Aldrich Product Number: SML3162). Nerol (CAS Number: 106-25-2, Sigma-Aldrich Product Number: 50949). Hydroquinone (CAS Number: 123-31-9, Sigma-Aldrich Product Number: 8.22333). 5-Fluoroindole-2-carboxylic acid (CAS Number: 399-76-8, Sigma-Aldrich Product Number: 265128). Myricetin (CAS Number: 529-44-2, Sigma-Aldrich Product Number: 89013). Fenoterol can be purchased as the hydrobromide salt (CAS Number: 1944-12-3, Sigma-Aldrich Product Number: F1016). Flunisolide (CAS Number: 3385-03-3, Sigma-Aldrich Product Number: F5021). Hydrocortisone (CAS Number: 50-23-7, Sigma-Aldrich Product Number: H0888). Hydrocortisone acetate (CAS Number: 50-03-3, Sigma-Aldrich Product Number: H1400000). Halcinonide (CAS Number: 3093-35-4, Sigma-Aldrich Product Number: 1302509). Budesonide (CAS Number: 51333-22-3, Sigma-Aldrich Product Number: PHR1178). Fluocinonide (CAS Number: 356-12-7, Sigma-Aldrich Product Number: 1276001). Fluocinolone acetonide (CAS Number: 67-73-2, Sigma-Aldrich Product Number: 1275009). Clobetasol propionate (CAS Number: 25122-46-7, Sigma-Aldrich Product Number: PHR1921). Prednisolone (CAS Number: 50-24-8, Sigma-Aldrich Product Number: PHR1043). Triamcinolone (CAS Number: 124-94-7, Sigma-Aldrich Product Number: 1676000). Triamcinolone diacetate (CAS Number 67-78-7, Sigma-Aldrich Product Number: 1678005). Nitrofurazone (CAS Number: 59-87-0, Sigma-Aldrich Product Number: 1465004). Benseraside hydrochloride (CAS Number: 14919-77-8, Sigma-Aldrich Product Number: B7283).

Bone marrow-derived macrophages: Bone marrow-derived macrophages (BMDMs) were harvested from the femurs of 6-week-old C57Bl/6J mice as reported previously. 21 They were plated at a density of 50×106 cells/10 cm dish in primary culture medium: RPMI 1640 (Life Technologies), 10% heat inactivated fetal bovine serum (HIFBS), 2×10-3 M Lglutamine (Life Technologies), antibiotic antimycotic (1×) (Life Technologies), and 10% MCSF (Mycoplasma free L929 supernatant) for 5 days at 37° C. and 5% CO₂. On day 6 of culture, cells were detached with 5 mM EDTA in PBS, counted, and plated for further assays.

High-throughput trained immunity screening: Mature BMDMs were plated at 25,000 cells per well in 45 μL media in 384-well flat-bottomed plates using the Thermo Multidrop Combi liquid handler. Cells were rested overnight at 37° C. and 5% CO₂. The following day, training compounds from the Microsource Spectrum Library were added at a volume of 45 nL using the PerkinElmer Janus G3 with Pintools for a final concentration of 1 μM. On day 2, supernatant was removed using the Janus, cells were washed with warm PBS using the Multidrop, and fresh media was added to the cells using the Multidrop. On day 5, cell density was quantified using the IncuCyte imager and software, then cells received media containing 25 ng/mL IFN-7 (BD Biosciences) for 24 hours. On day 6, cells were stimulated with 25 ng/mL Pam3-CSK4 (Invivogen). Supernatant was collected on day 6 and on day 7 following 24 hours of TLR stimulation, and it was stored at −80° C.

To measure TNF-α concentration in the supernatant, a high-throughput Quanti-Blue assay was used. HEK-Blue TNF-α cells (Invivogen) were plated at a density of 25,000 cells per well in 45 μL media (DMEM, 10% HIFBS). 5 μL of supernatant is added to each well using the Janus liquid handler, then the plates were incubated for 24 hours at 37° C. and 5% CO₂. The following day, 20 μL of concentrated Quanti-Blue reagent was added to each well using the Combi liquid handler, and the plate was shaken for one minute. Then, the plate incubated at room temperature for 15 minutes before absorbance was measured at 628 nm using the BioTek Synergy NEO 2. All data is represented as a fold change relative to the average of the Pam3 only untrained control (n=20) on each plate.

In vitro trained immunity assay: Mature BMDMs were plated at 105 cells per well in 200 L media in 96-well flat-bottomed plates and rested overnight at 37° C. and 5% CO₂. The following day, training molecules were added at specified concentrations and incubated for 24 hours. After training, cells were washed with warm PBS and rested for 3 days in fresh media. On day 5, cells received fresh media containing 25 ng/mL IFN-7 (BD Biosciences) for 24 hours. On day 6, cells were stimulated with 100 ng/mL Pam3CSK4 (Invivogen). Cell supernatant was collected 24 hours after stimulation. Supernatants were measured for IL-6 and TNF-α concentrations using ELISA MAX Deluxe kits (BioLegend) according to manufacturer's instructions.

For inhibition studies, BMDMs received inhibitors at specified concentrations 1 h prior to stimulation with training molecules, and inhibitors were present for the entire 24 h training period. To test epigenetic pathway involvement, 5′-deoxy-5′-(methylthio)adenosine (MTA) and (-)-epigallocatechin-3-gallate (EGCG) were used at final concentrations of 500 μM and 15 μM, respectively. To test metabolic pathway involvement, cells were pretreated with 1 mM 2′ deoxy-D-glucose.

In vivo trained immunity assay: C57Bl/6J mice were trained via intraperitoneal injection (IP). For each compound, mice were treated with 1.5-2 μmol of the indicated small molecule in 50 L (10% DMSO and 90% Corn Oil) every other day for a total of 3 training injections. Mice were rested for 7 days following the final injection, and then challenged IP with 5 μg LPS (serotype 055:B5; Invivogen) in 100 μL PBS. Mice were bled via the submandibular vein 1 h following LPS injections. Serum was prepared by allowing the blood to clot for 30 minutes, then centrifuged for 15 minutes at 2000×g. Serum cytokines were quantified via ELISA (Biolegend) for IL-6 and TNF-α according to manufacturer instructions.

ATAC-seq: Mature BMDMs were plated at 105 cells per well in 200 μL media in 96-well flat-bottomed plates (Corning) and rested overnight at 37° C. and 5% CO₂ and then trained as described above. On day 5, instead of pre-treatment with IFN-γ, cells instead received fresh media. On day 6, cells were detached from the plate in lysis buffer (10 mM Tris-HCl, pH 7.4, 10 mM NaCl, 3 mM MgCl2, 0.1% IGEPAL CA-630 (abeam)) and transferred to 96-well v-bottomed plates (Corning), then centrifuged at 500 G for 5 minutes. The supernatant was removed with a multichannel pipette and the nuclei pellets were resuspended in 50 μL of transposition buffer (25 L 2×D Buffer (Illumina), 2.5 μL Tn5 Transposes (Illumina), 22.5 μL Nuclease Free H2O). The plate was incubated at 37° C. on a shaker set to 800 rpm. Then, transposed DNA was purified using a Qiagen MinElute kit according to manufacturer instructions and frozen at −80° C.

The following day, samples were thawed and amplified using PCR. The reaction was monitored with qPCR to prevent CG bias and oversaturation. After PCR, the samples were again purified using the Qiagen MinElute kit and analyzed via Agilent High-Sensitivity DNA Bioanalyzer. Samples were then stored at −80° C. When the samples were ready to be sequenced, each sample was diluted to a final concentration of 20 nM and pooled for sequencing.

ATAC-seq Analysis: Statistical analysis was performed using GraphPad Prism (v8). Data were analyzed using one- or two-way analysis of variance with multiple comparisons. Direct comparisons were made using the 2-tailed student's t-test. Data are reported as mean+/−standard deviation. Number of replicates is reported in the figure descriptions. For in vitro experiments, n represents the number of technical replicates, while for in vivo or ex vivo experiments, n represents the number of animals.

### Example 1

### High-Throughput Screen Identifies Novel Inducers of Trained Immunity

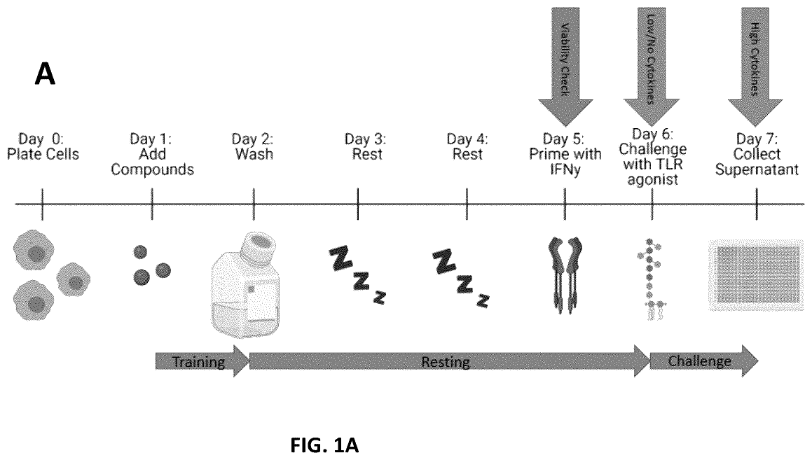

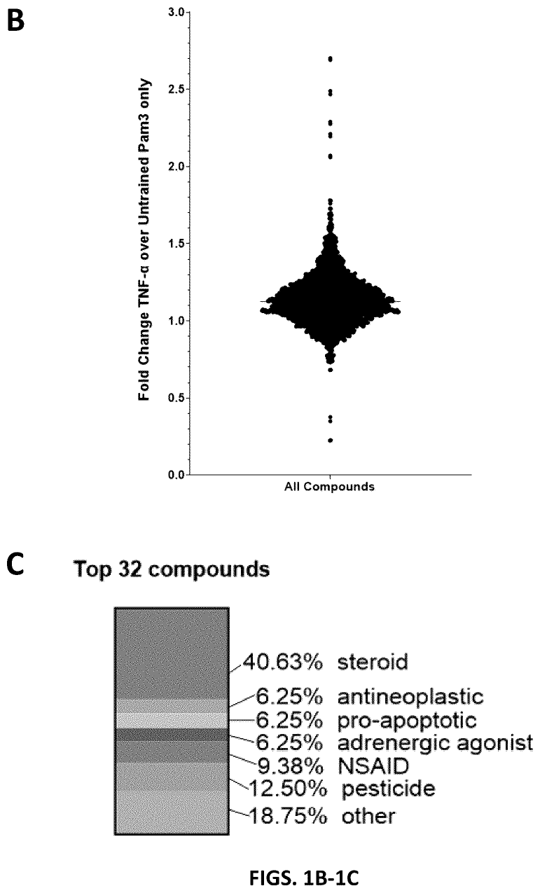

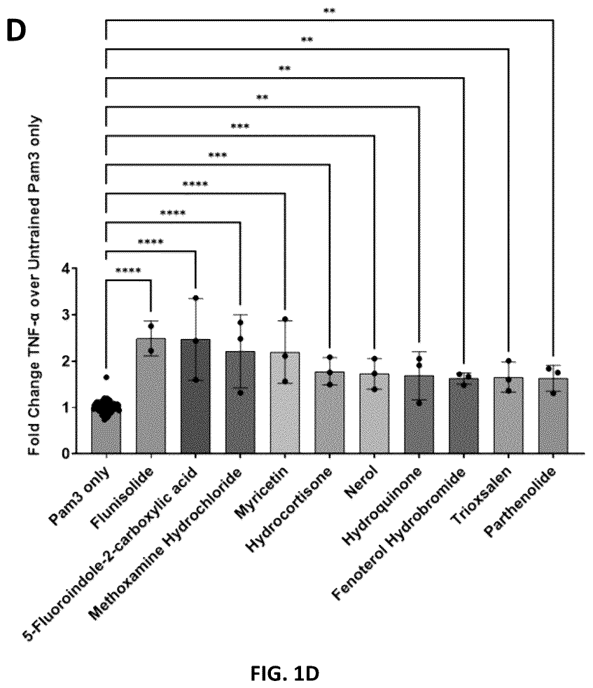

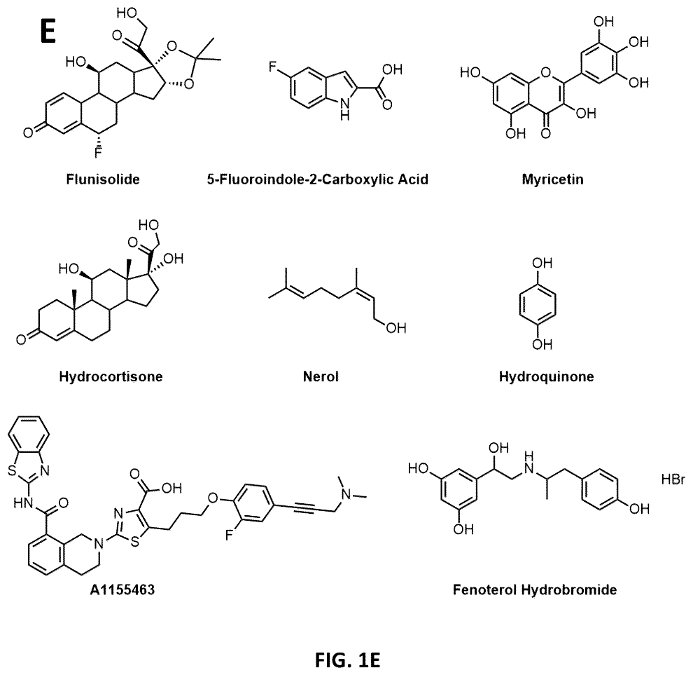

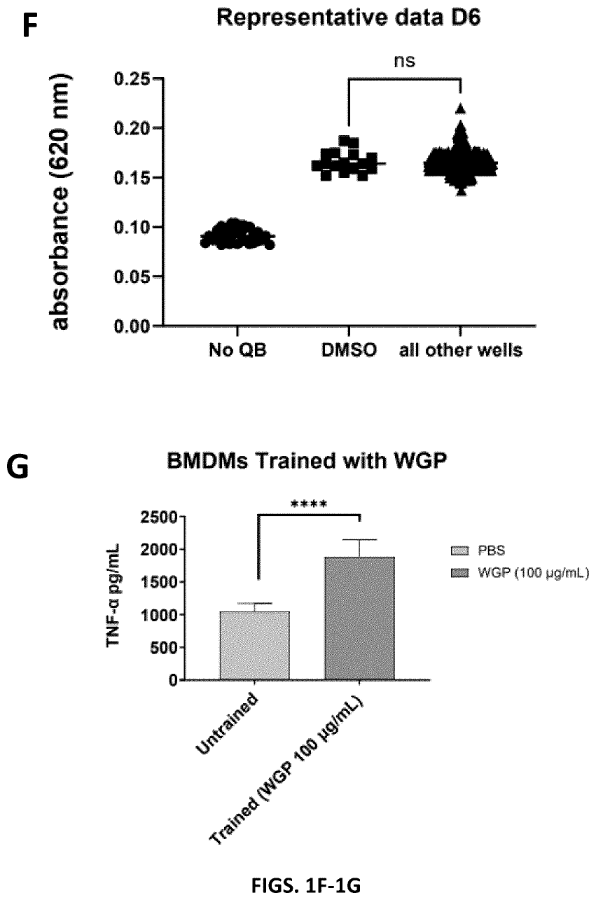

To achieve an effective high-throughput screen for in vitro induction of training, several technical challenges were addressed. Previous experience with high-throughput screening to identify immunomodulators provided insight into the required points of optimization.¹⁹ First, the screen involved the use of primary cells. Thus, the method was developed to enhance the consistency of the assay and the viability of the cells. An additional plate was seeded alongside the screen plates for a manual training assay and ELISA measurement of cytokine secretion to confirm that the BMDMs were behaving as expected. The full screening protocol is represented in FIG. 1A. On Day 5, each well was imaged using an IncuCyte to quantify cell viability relative to the untrained controls.²⁰ Furthermore, the cells were checked to see if they maintained a resting phenotype before TLR stimulation by collecting and freezing the supernatant on Day 6 to measure TNF-α concentration after the assay was complete. Trained immunity effects are distinct from innate immune priming, so we checked that the cells maintained a resting phenotype before TLR stimulation by collecting and freezing the supernatant from each of the three screening replicates on Day 6 to measure TNF-α concentration after the assay was complete (FIG. 1F). With these technical and biological controls providing reproducible confirmation of training with β-glucan, we applied this screening methodology to identify compounds that could induce a phenotype matching that produced by β-glucan, analogous to training, in vitro.

With these technical checkpoints, it is believed that a screen using this methodology successfully identifies compounds that induce trained immunity in vitro.

For each replicate of the screen, a modified version of the standard 7-day training assay was used.¹⁸ TNF-α concentration was measured using a high throughput Quanti-Blue assay in HEK-Blue TNF-α cells. Concentration is proportional to absorbance measured at 628 nm within the range of the assay. 2,000 small molecules from the MicroSource Spectrum Library were screened. A hit was defined as exhibiting a 1.6-fold or greater increase in TNF-α production over stimulated, untrained controls as measured by the high-throughput Quanti-Blue-based assay. During screening optimization, β-glucan exhibited a 1.8-fold increase in TNF-α production over untrained controls, which was consistent with our previous work.²¹ A slightly lower cutoff was selected for hit validation to address the possibility that the library concentration of 1 μM was not optimal for every compound tested. This cutoff is also 2.5 standard deviations from the mean of all library compounds. 32 compounds were identified over the 1.6-fold increase threshold, representing 1.5% of the compounds tested. See Table 1, below, where “Fold Change” refers to TNF-α production increase over untrained control. See also FIG. 1B. These compounds clustered into several classifications including glucocorticoids, which made up 18 of the top 24 compounds, as well as adrenergic agonists, pesticides, and antineoplastic drugs. See FIG. 1C. The 7 top performing compounds were selected (including representative compounds from each classification) for further investigation in vitro and in vivo. See FIGS. 1D and 1E.

TABLE 1
Small Molecule Inducers from HTS.

| Compound | Fold Change | Compound Class |
|---|---|---|
| Halcinonide | 2.702 | steroid |
| Budesonide | 2.690 | steroid |
| Flunisolide | 2.489 | steroid |
| 5-Fluoroindole-2-carboxylic | 2.468 | NMDA receptor antagonist |
| acid | | |
| Fluocinonide | 2.289 | steroid |
| Fluocinolone acetonide | 2.276 | steroid |
| Methoxamine hydrochloride | 2.211 | alpha-1 adrenergic agonist |
| Myricetin | 2.195 | flavonoid/antioxidant/ |
| | | topoisomerase inhibitor |
| Clobetasol propionate | 2.070 | steroid |
| Prednisolone | 2.059 | steroid |
| Mechlorethamine | 1.781 | antineoplastic |
| Hydrocortisone | 1.778 | steroid |
| Triamcinolone | 1.760 | steroid |
| Triamcinolone diacetate | 1.727 | steroid |
| Nerol | 1.726 | monoterpenoid alcohol |
| Zomepirac Sodium | 1.699 | NSAID |
| Nitrofurazone | 1.691 | steroid |
| Hydrocortisone acetate | 1.684 | steroid |
| Hydroquinone | 1.683 | topical skin bleaching agent |
| Oxyphencyclimine | 1.673 | anticholinergic |
| hydrochloride | | |
| Isokobusone | 1.662 | anti inflammatory |
| Trioxsalen | 1.657 | psoralen, photosensitizer |
| Toxaphene | 1.634 | insecticide |
| Parthenolide | 1.632 | anti inflammatory |
| Fenoterol hydrobromide | 1.622 | beta-2 adrenergic agonist |
| Simvastatin | 1.611 | statin |
| Dichlorophene | 1.611 | laxative vermicide |
| Benserazide hydrochloride | 1.610 | steroid |
| Tetrachloroisophthalonitrile | 1.609 | fungicide |
| Nateglinide | 1.605 | meglitinide/T2D drug |
| N-Acetylaspartic acid | 1.603 | modifies lipid synth. and |
| | | acetylation |
| Floxuridine | 1.603 | antineoplastic |

### Example 2

### Non-Immunogenic Small Molecules Suppress Acute Inflammation but Induce a Training Phenotype

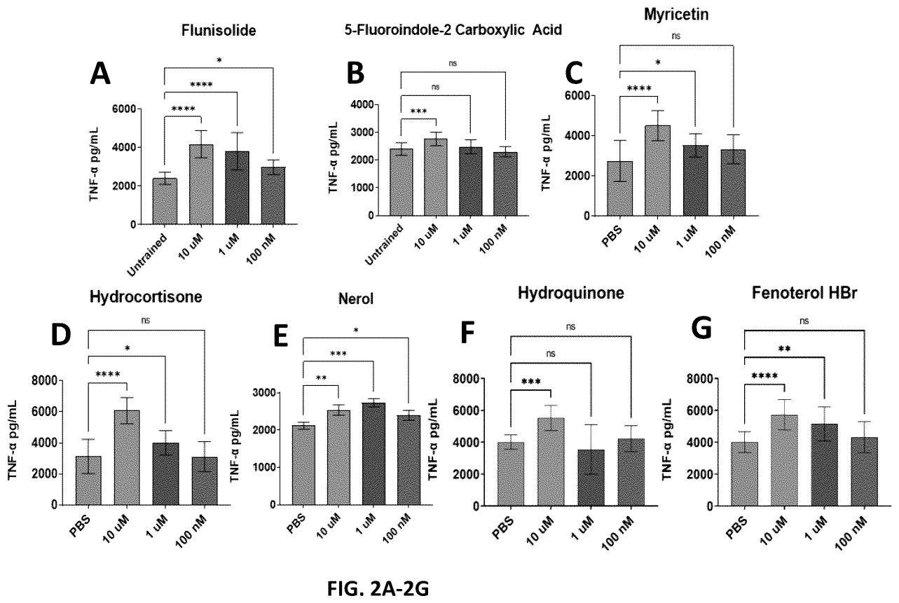

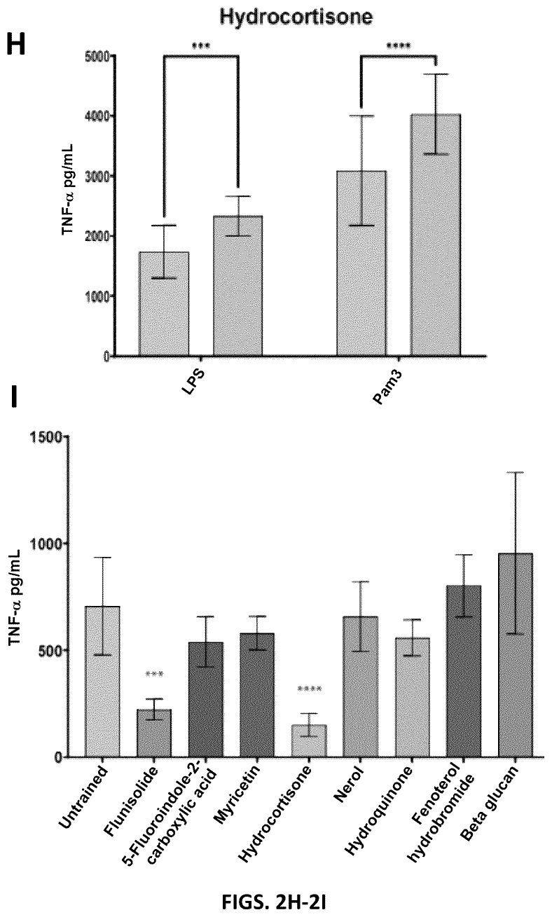

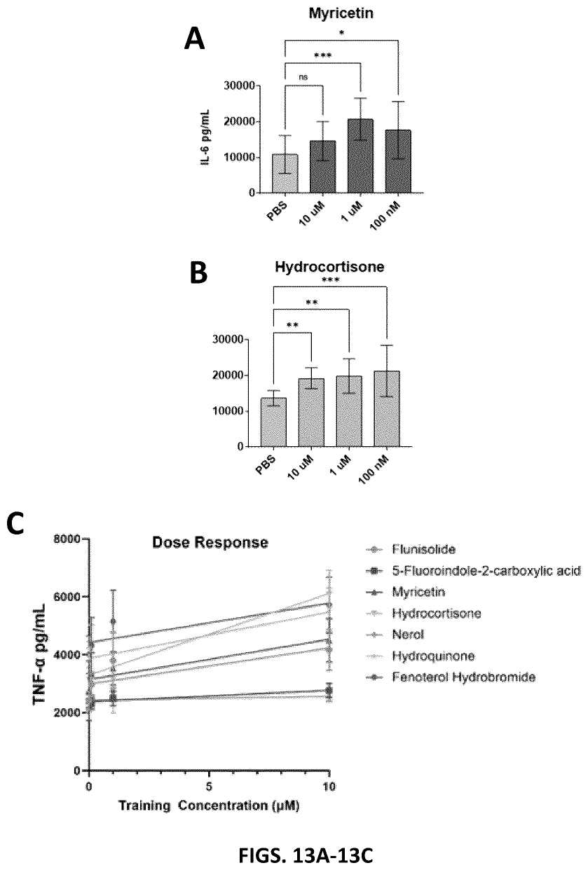

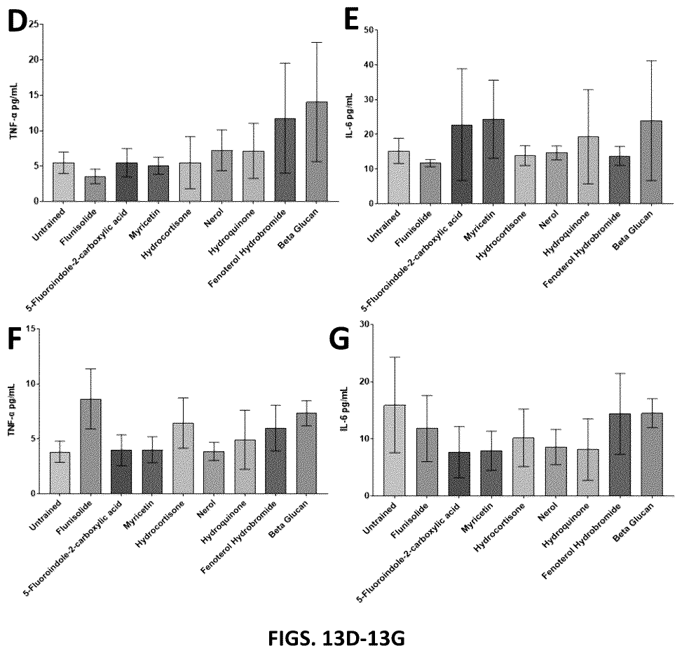

Having identified a set of compounds for further study, the compounds' activities were validated and stimulatory characteristics determined through further in vitro study. To select an optimal concentration and agonist pairing, a series of ELISAs were performed to validate the training effects. BMDMs were trained with hit compounds at multiple concentrations (100 nM-10 μM) to identify the most effective concentrations. See FIG. 2A. 10 μM was found to be the most effective across all hit compounds and was therefore selected for all subsequent in vitro experiments. In each experiment, the elevation of both TNF-α and IL-6 was observed. Most compounds elicited a significant, positive dose-dependent training response, and 10 μM was the most effective at inducing upregulation of TNF-α across most hit compounds (FIG. 13C). A hallmark of trained immunity is an increased cytokine response to heterogeneous challenges, providing broad protection against multiple pathogens.²² To demonstrate that effective training responses are compatible with multiple TLR agonists, BMDMs were stimulated with either Pam3CSK4 (a TLR1/2 agonist) or LPS (a TLR4 agonist). See FIG. 2B. Unlike β-glucan and BCG, these hit compounds do not induce cytokine production on their own. In fact, some compounds suppress acute inflammation when given 1 hour before stimulation with LPS. See FIG. 2C. It was also confirmed that the response is not due to innate immune priming by testing the supernatant taken prior to TLR stimulation. The supernatant collected on Day 6 displayed negligible pro-inflammatory cytokine production. In contrast to known inducers of training, these compounds were unique in their inflammatory signatures, so further studies were performed to discern any pathway involvement of known metabolic and epigenetic characteristics.

### Example 3

### In Vitro Chemical Inhibition of Glycolysis and Epigenetic Modification

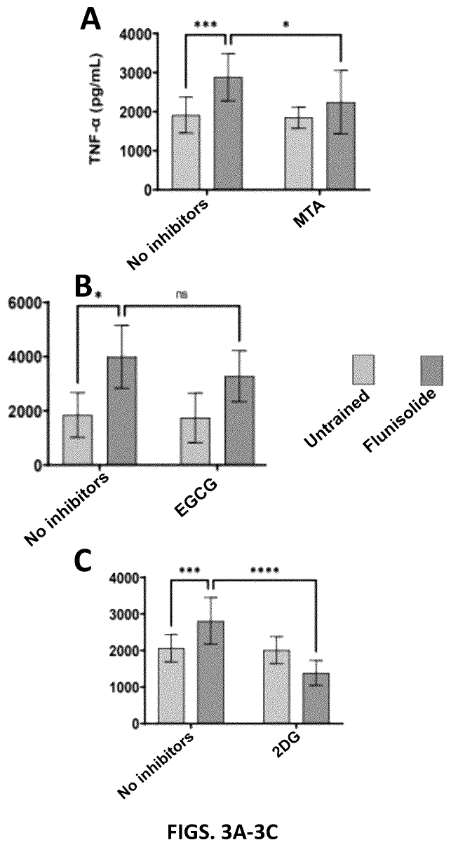

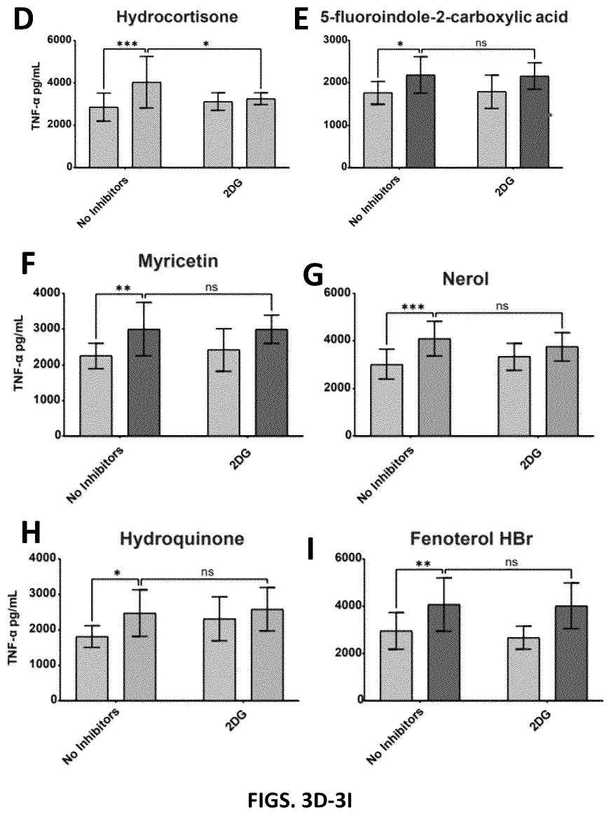

Upregulation of glycolysis is commonly implicated as one of the underlying mechanisms in the induction of trained immunity. Representative top performing compounds were tested using a standard set of glycolytic and epigenetic inhibitors of training given 1 hour before small molecule administration.^{18,23} In this study, a reduction in cytokine elevation over untrained controls indicates glycolytic or epigenetic involvement. In one of the presently identified inducers of trained immunity, flunisolide, significant inhibition of training was observed in response to a methyltransferase inhibitor (MTA) and a glycolysis inhibitory (2-deoxy-D-glucose). See FIG. 3. Treatment with a histone acetyltransferase inhibitor (EGCG) appeared to slightly reduce training though statistical significance was not achieved. See FIGS. 3A to 3C. The untrained control did not respond to treatment with inhibitors, as expected. Glucocorticoids flunisolide and hydrocortisone, we observed significant inhibition of training in response to glycolysis inhibition (2-deoxy-D-glucose) (FIGS. 3D and 3E). However, all other compounds besides glucocorticoids did not result in inhibition of glycolytic training pathway in the assay (FIGS. 3F to 3I). The untrained control did not respond to treatment with inhibitors, as expected. These results suggest that glucocorticoids induce trained immunity through glycolytic and epigenetic pathways and that novel pathways may be involved in training with other compounds.

### Example 4

### Non-Inflammatory Small Molecules Induce Trained Immunity Responses In Vivo

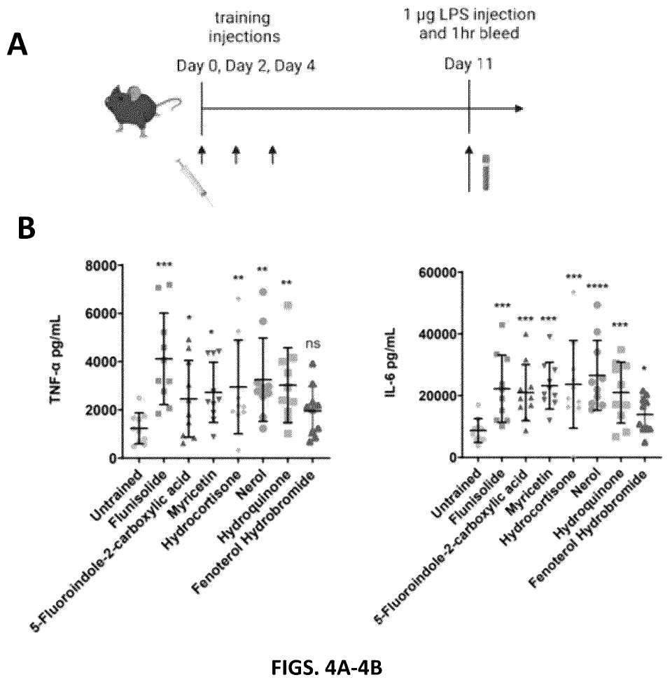

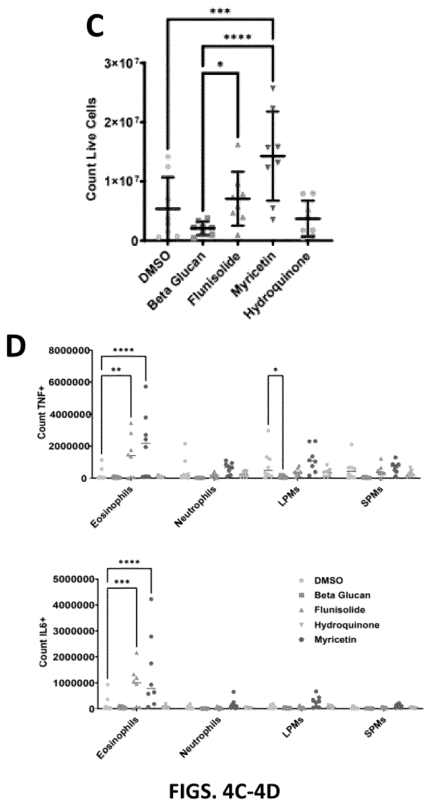

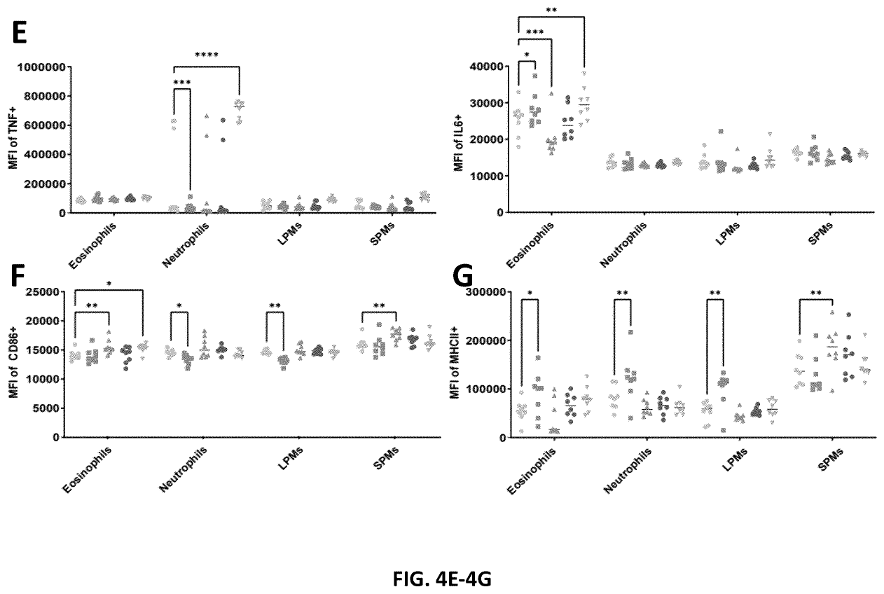

To test the ability of the small molecules in inducing trained immunity in vivo, 3 training injections were given intraperitoneally (IP) every other day and then the mice were allowed to rest for one week. After the rest period, the mice were challenged with 1 μg LPS IP. See FIG. 4A. Serum was collected after 1 hour and tested for pro-inflammatory cytokine responses. Compared to the untrained control, each of the 7 compounds selected for validation in this study exhibited elevated TNF-α and IL-6 serum cytokines. See FIG. 4B.

The cells and cell types that were contributing to the increase in serum cytokines from training with the small molecules were investigated. Three of the most promising compounds, flunisolide, myricetin, and hydroquinone, were selected to investigate the cellular response. To determine which cells were involved, mice were given training injections and challenged as previously described. Then, 2 h after challenge, the mice were sacrificed, and peritoneal cells were collected by lavage. The cells were fixed and stained for intracellular TNF-α and IL-6 and surface costimulatory markers, CD40, CD86, and MHCII. Lineage markers were included for innate immune cells, including neutrophils, eosinophils, monocytes, macrophages, and dendritic cells. The stained cells were then analyzed via spectral flow cytometry. Initial observations indicated that mice trained with flunisolide or myricetin exhibited increased cell count in the peritoneal lavage when compared to untrained or β-glucan trained controls (FIG. 4C) The increases in cell numbers were distinctive for each training compound. Flunisolide- and myricetin-trained mice exhibited significant increases in the number of TNF-α+ and IL-6+ eosinophils (FIG. 4D). Mice trained with hydroquinone presented a different phenotype. While there was no increase in the number of TNF-α+ and IL-6+ cells, there were significant increases in the cytokine MFIs among TNF-α+ neutrophils and IL-6+ eosinophils in the hydroquinone-trained mice (FIG. 4E). These results suggest that flunisolide and myricetin promote a stronger production of TNF and IL-6 via the increased number of eosinophils generating these cytokines. Alternatively, hydroquinone induces greater production of TNF-α and IL-6 in neutrophils, but not significantly greater number. This contrasts with β-glucan which does not generate a greater number of cells in the peritoneal space, but still resulted in higher MFI of IL-6 in eosinophils.

The activation of these cells and changes to co-stimulatory molecules were then investigated. When considering stimulatory surface molecules such as CD86 and MHCII, only flunisolide-trained mice were significantly different from controls. Flunisolide-trained mice exhibited an increased amount of CD86 on eosinophils and small peritoneal macrophages (SPMs), as measured by MFI of CD86+ cells (FIG. 4F). Additionally, the MFI of MHCII+ SPMs was also increased in mice trained with flunisolide (FIG. 4G). 0-glucan-trained mice exhibited increased MFI of MHCII+ eosinophils, neutrophils, and large peritoneal macrophages (LPMs). Taken together, these data indicate that while there are similar increases in serum cytokines in small molecule trained mice, there are differences in the cellular profile of the innate cells that respond to LPS challenge. Thus, there may be differences in the cellular mechanisms by which each small molecule induces trained immunity in vivo.

### Example 5

### ATAC-Seq Reveals Differential Chromatin Accessibility Between Small Molecule Trained BMDMs

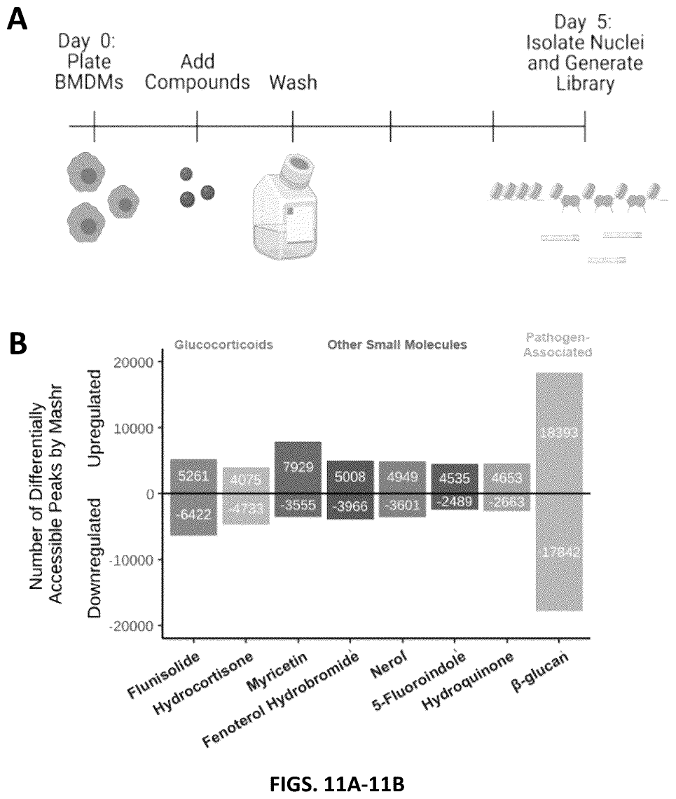

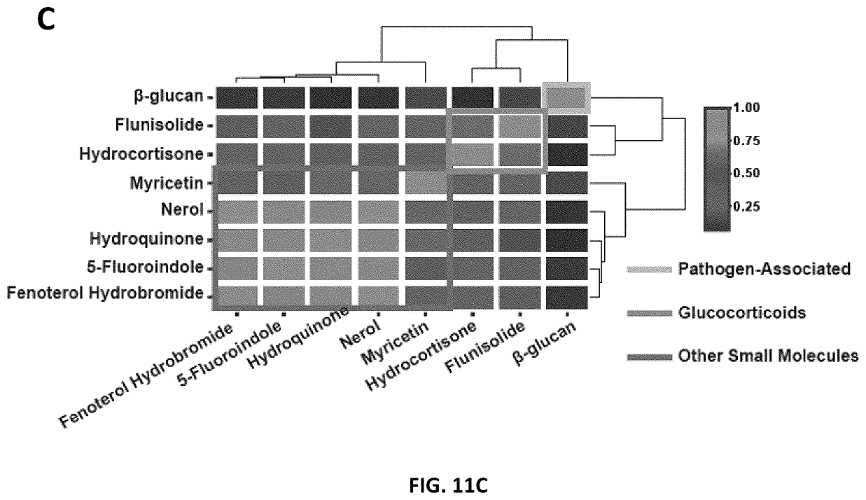

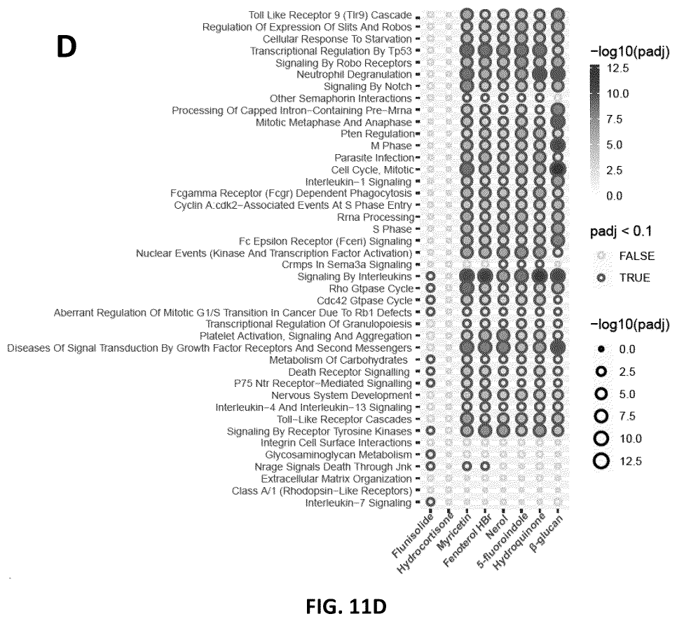

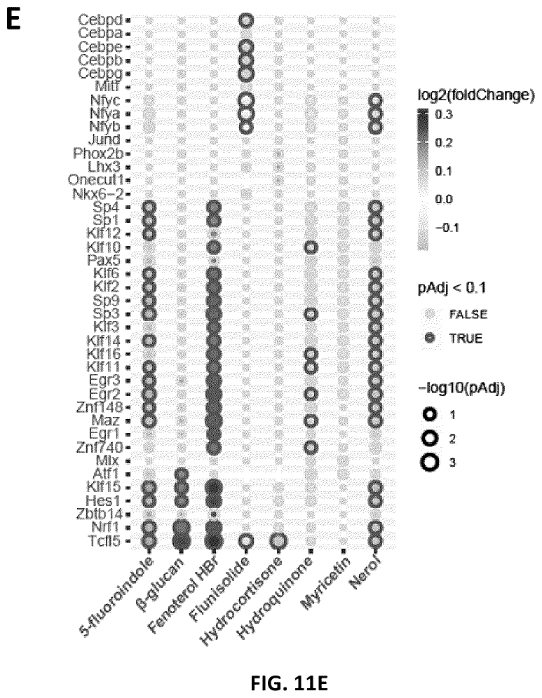

Training with distinct small molecules could induce alterations in chromatin accessibility, and these changes could potentially vary depending on the specific stimulus from each training compound. To rigorously evaluate this hypothesis, nuclei from BMDMs were isolated four days after their exposure to the top seven hit compounds and conducted ATAC-seq experiments (FIG. 11A). Subsequently, regions of open chromatin peaks were identified that exhibited significant alterations in accessibility levels in response to the diverse treatments. To enhance statistical power and precision in estimating effect sizes for these “Differentially Accessible (DA) peaks,” a multivariate adaptive shrinkage method known as ‘mash’, was employed which capitalizes on the correlation structure of effect sizes across the various treatments.

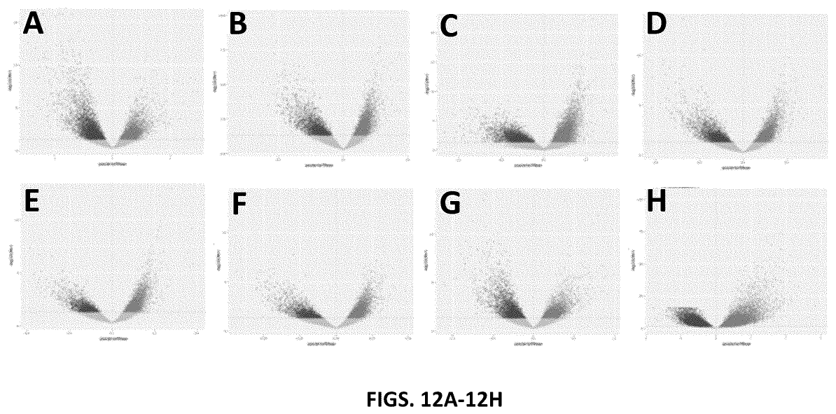

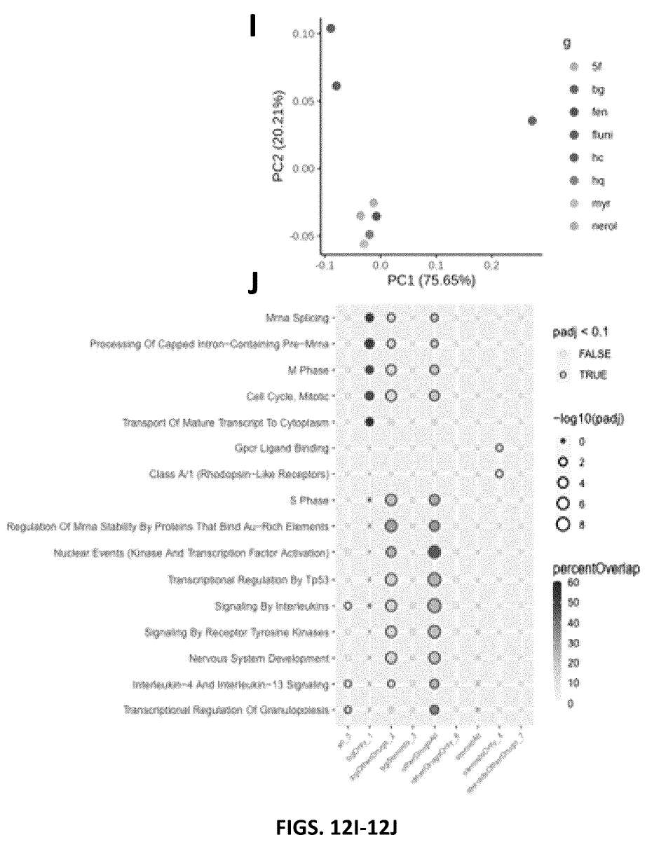

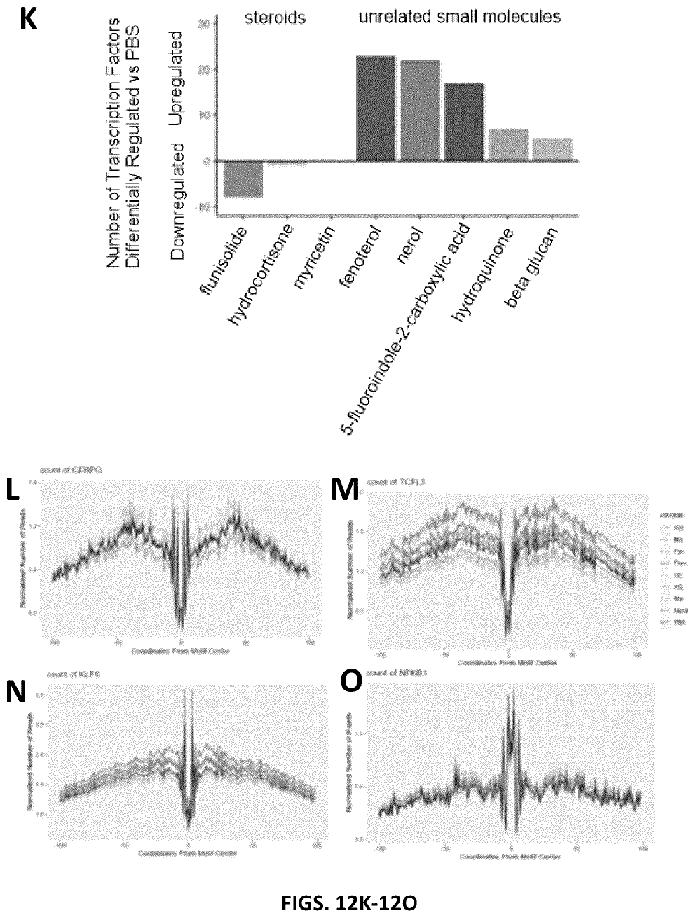

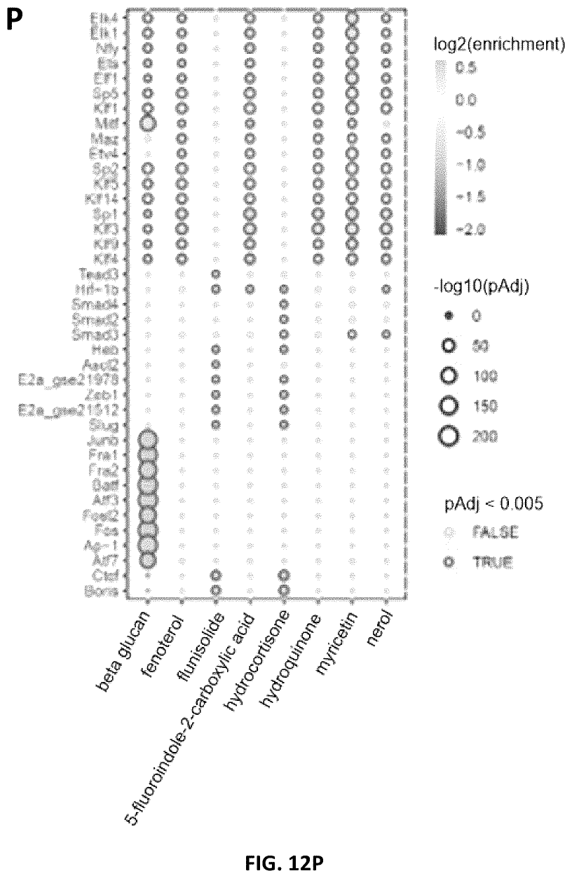

The greatest number of DA peaks was observed in macrophages treated with β-glucan (comprising 36,235 peaks classified as DA at a False Discovery Rate (FDR)<10%), followed by Flunisolide (n=11,683 DA peaks) (FIGS. 11B and 12A-12H). Conversely, all other treatments resulted in a lower number of DA peaks, with the fewest changes being observed in response to Hydroquinone (n=7,316 DA peaks). A correlation analysis of effect sizes across treatments revealed three distinct clusters: cluster 1 encompassed β-glucan, cluster 2 included glucocorticoids, while cluster 3 incorporated all remaining treatments (FIGS. 11C and 12I). This observation indicates that the pattern of chromatin alterations linked to functional training diverges based on the specific stimulus. This analysis of differential accessibility unveiled distinct epigenetic signatures unique to glucocorticoids, pathogen-associated β-glucan, and training with other small molecules.

To further understand how these compounds influence transcriptional response, genes with the greatest accessibility both for individual molecules and for those commonly shared across all compounds including β-glucan were examined. These commonly shared alterations in chromatin accessibility are presented in FIG. 12J. Two of the most statistically significant peaks for which chromatin accessibility increased among all compounds tested localized near SH3GLB1 and SCARB1 transcription start sites. SH3GLB1 affects mitochondrial dynamics, autophagy, and apoptosis, and SCARB1 is a scavenger receptor involved in cholesterol uptake and metabolism. Gene set enrichment analysis identified a significant increase in chromatin openness (FDR<5%) nearby genes in several pathways thought to be important for trained immunity in each of the treatments tested. Such pathways include interleukin signaling, TLR signaling, phagocytosis, chromatin modification, and carbohydrate metabolism (FIG. 11D). When analyzing only the peaks that changed accessibility levels across all treatments, an enrichment of accessibility nearby genes related to IL-4 and IL-13 signaling pathways and transcriptional regulation of granulopoiesis was identified (FIG. 12J). Interestingly, a specific enrichment of GPCR ligand binding, and rhodopsin-like receptor activity (FIG. 12J) in glucocorticoid-trained BMDMs were identified, which implies that glucocorticoids induce changes that are distinct from, and in addition to, common training pathways.

Transcription factors (TFs) that might be associated with the observed changes in the epigenetic landscape of BMDMs treated with the different molecules were then investigated. Across all treatments, NF-κB, Jun, Fos, and CEBP binding sites were enriched among regions that gained chromatin accessibility, suggesting that the activation of these TFs in response to the different treatments is likely a driving force behind the epigenetic changes identified (FIG. 12P). Previous work in trained immunity has identified Jun and Fos as key transcriptional regulators of training. Additionally, increased motif enrichment of Krüppel-like factors (KLF) and Specificity protein (Sp) family TFs were identified, which are associated with cell proliferation and differentiation, in all treatments except for the glucocorticoids. Notably, KLF14, enriched in each of the non-steroid treatments, has been shown to regulate glycolysis in macrophages. Footprinting analysis was then performed to determine if there were differences in bound TFs between the training molecules in the trained, unstimulated state. This information may provide insights into the roles of TFs in the induction of the trained state. There was a decrease in actively bound TFs in glucocorticoid-trained BMDMs, whereas training with the other small molecules or β-glucan increased the number of bound TFs relative to PBS-treated BMDMs (FIG. 12K). Many of the KLF and Sp family TFs were identified in footprinting analysis and share consensus motifs, though many of these TFs compete for the same binding sites and have both repressor and activator functions (FIGS. 11E and 12A to 12H). Unsurprisingly, neither NF-κB nor its subunits showed evidence of occupancy in the footprinting analysis, emphasizing that the trained, unstimulated BMDMs do not promote constitutive inflammatory activation (FIGS. 12A to 12H). These findings collectively suggest that training with distinct small molecules can induce both the fundamental chromatin remodeling necessary for initiating training across all treatments and distinctive epigenetic changes likely mediated by the activation of distinct sets of transcription factors as implied by the footprinting analysis. The unique patterns of activation of trained immunity demonstrated by the glucocorticoids and other novel small molecule inducers of training deserves further mechanistic study.

### Example 6

Trained Immunity with A1155463

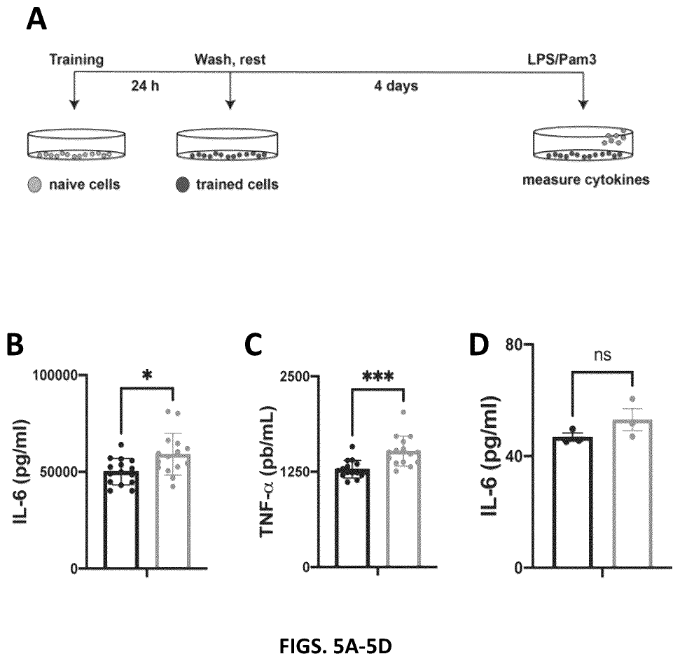

A1155463 is a commercially available inhibitor of the B-cell lymphoma-extra large (Bcl-xL) protein. To test if A1155463 induced training, an in-vitro training assay was performed in BMDMs. See FIG. 5A. In this assay, BMDMs were trained with either PBS (untrained or control group) or A1155463 at various concentrations for 24 hours. After 24 hours, the cells were washed, and the media was replaced. The cells were allowed to rest for four days. On day 6, cells were washed and challenged with Pam3 (TLR 1/2 agonist). The supernatant was tested 24 hours following the challenge. Trained cells produce a higher concentration of pro-inflammatory cytokines, e.g., IL-6 and TNF-α, than untrained cells following challenge with a TLR agonist. An approximately 1.2-fold increase was observed in both IL-6 and TNF-α following the challenge with Pam3. See FIG. 5B.

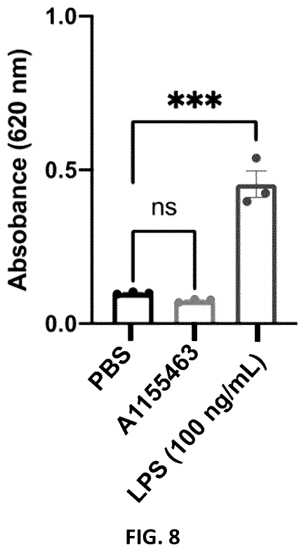

Bcl-xL is an anti-apoptotic protein with some role in mediating inflammatory reactions.^{32,33} Most documented inducers of trained immunity elicit an initial immune response upon administration. However, A1155463 has never been reported elicit inflammatory responses in vitro. To test if A1155463 elicited an enhanced innate response on its own in vitro, supernatant from BMDMs cultured with A1155463 for 24 hours was analyzed. No significant difference in IL-6 levels between PBS-treated and A1155463-treated BMDMs was observed after 24 hours. See FIG. 5C. The non-immunogenicity of A1155463 was validated using an NF-kb reporter cell line. No significant difference between PBS-treated and A1155463-treated RAW-Blue™ cells (Invivogen) was observed after 18 hours (FIG. 8). Taken together, this evidence supports that A1155463 is a non-immunogenic trained immunity inducing molecule.

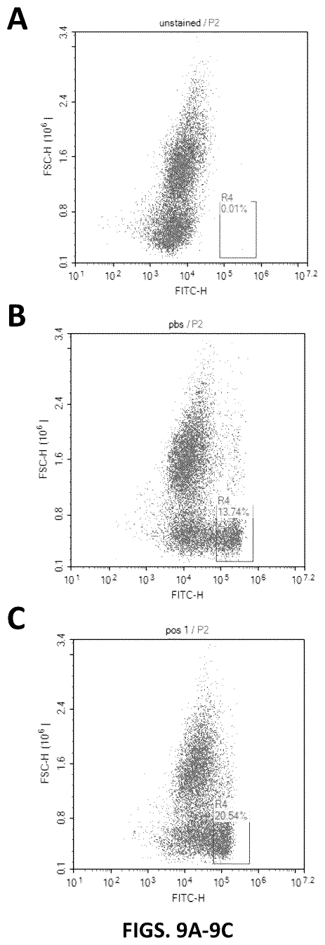

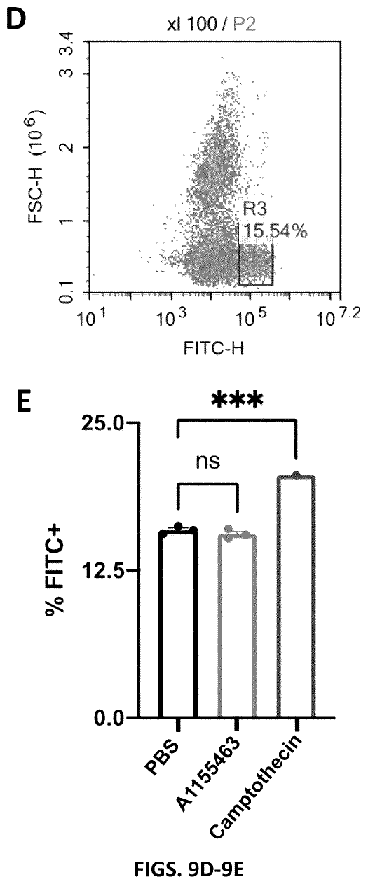

Since Bcl-xL is a known anti-apoptotic protein, a study was performed to see if inhibition with 100 nM A1155463 induced apoptosis in BMDMs. To test this, Annexin-V staining was used to gate for apoptotic cells 24 hours following stimulation with either PBS or A1155463. No significant difference was observed in the percentage of apoptotic cells in A1155463 treated cells (FIG. 9). Without being bound to any one theory, this result suggests that A1155463-induced training does not operate through an apoptotic mechanism.

After ruling out apoptosis and innate inflammation, an additional study was performed to attempt to determine the mechanism of action of A1155463-induced training in BMDMs. Training induces epigenetic and metabolic rewiring of macrophages which can be probed using small molecule inhibitors of various pathways.¹⁶ Epigenetic changes are associated with increased histone methylation and acetylation, promoting transcription and translation of proinflammatory cytokines in response to pathogenic stimuli. Metabolic changes alter cellular metabolism, mostly favoring glycolysis, to meet the higher energy requirements of a trained cell. In this study, BMDMs were pre-treated with small molecule inhibitors, namely, MTA (histone methyl transferase inhibitor), EGCG (histone acetyltransferase inhibitor), or 2-deoxy glucose or 2-DG (glycolysis inhibitor) for 30 min. This was followed by the standard training assay as described in FIG. 5A. For epigenetic small molecule inhibitors, no significant difference was observed. However, with the glycolysis inhibitor 2DG, pre-treatment caused a 50% reduction in the TNF-α level following Pam3 challenge. See FIG. 5D. A significant decrease in Pam3 induced IL-6 levels was also noted for different concentrations of 2-DG. See FIG. 5E. Without being bound to any one theory, this result suggests that A1155463 induced training likely proceeded through metabolic changes in BMDMs. This matches the previous literature describing Bcl family proteins that modulate cellular metabolism.³⁴⁻³⁷ Of note, to observe this inhibition of glycolysis, it was necessary to increase the concentration of 2DG from 1 mM to between 2 and 4 mM. This result suggests that glycolytic inhibition and therefore glycolysis can vary between inducers of trained immunity. See FIG. 5F.

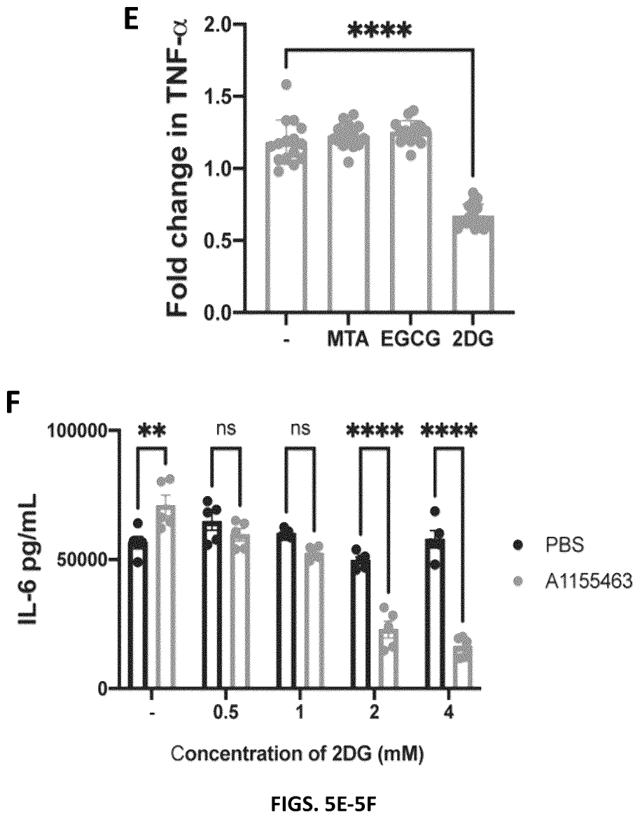

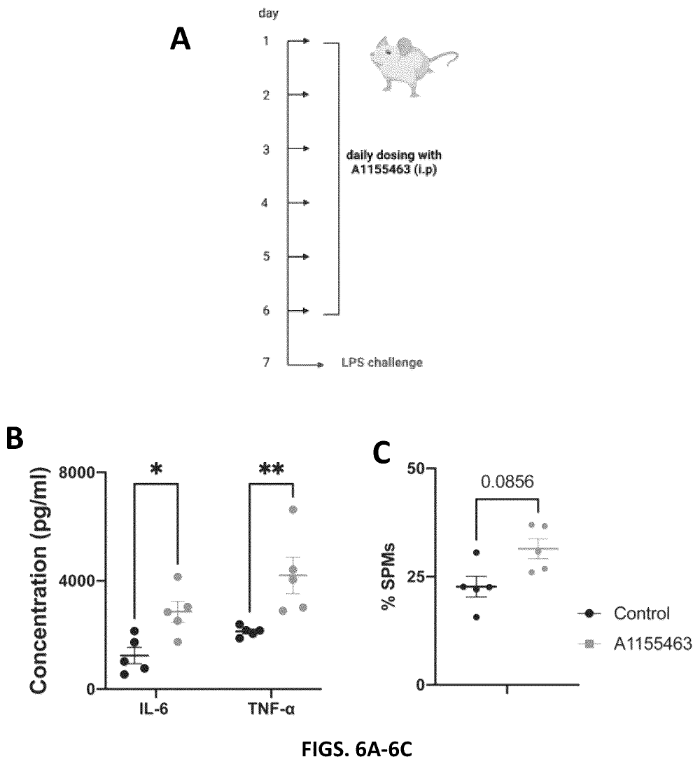

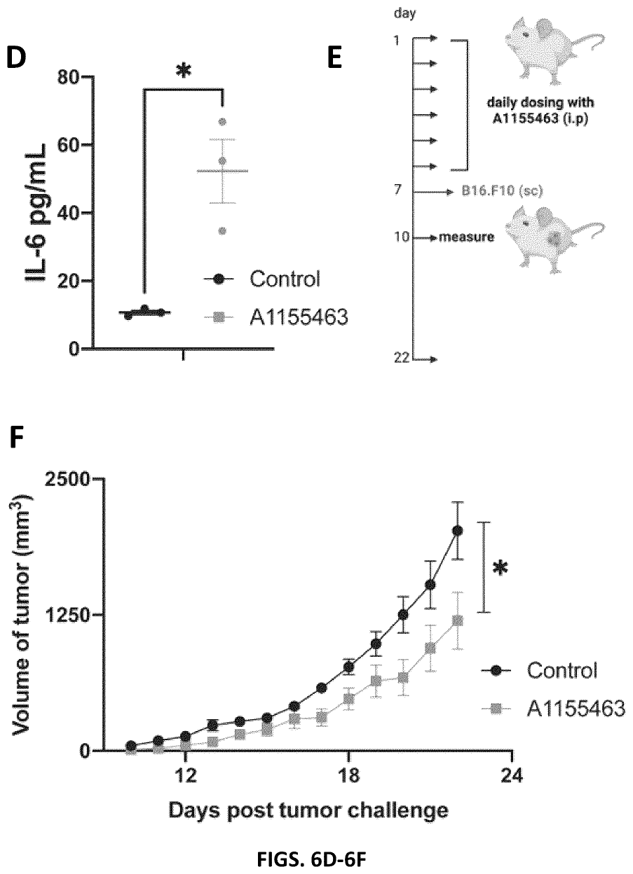

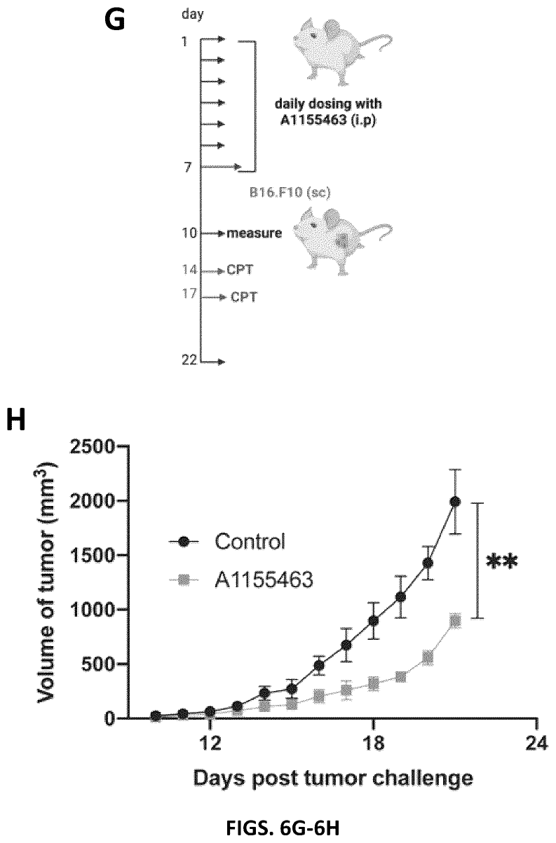

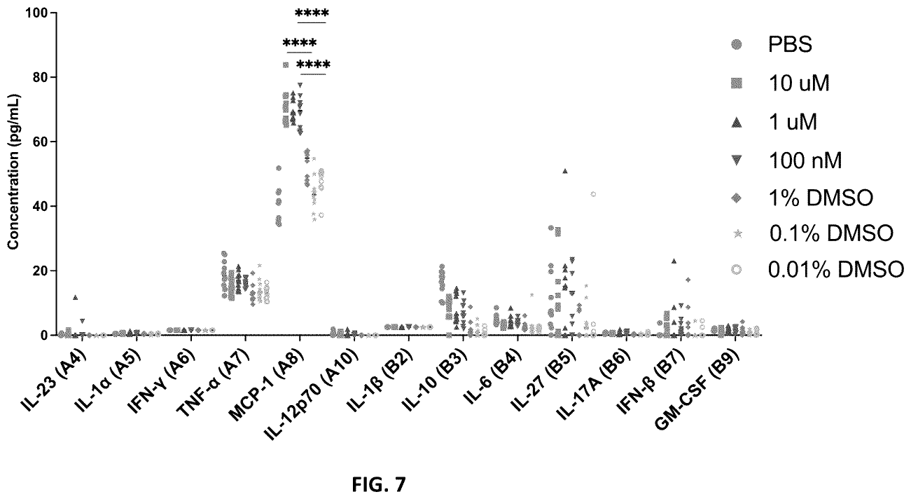

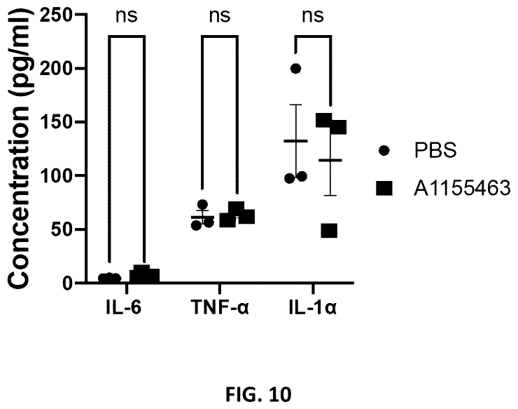

Since A1155463 induced training effects in-vitro, its ability to induce training in mice was studied. The half-life of Bcl-xL inhibitors in mice was previously determined to be around 9 hours.³⁸ To ensure sufficient material for training in vivo, a dosage of 15 mg/kg was administered intra-peritoneally daily for 6 days. See FIG. 6A. The vehicle control group was also dosed similarly to account for solvent effects. To rule out the impact of innate immune priming due to the high dosage, systemic cytokines were checked for 24 hours after the last dosing on day 6. No systemic IL-6 or TNF-α was observed, indicating no priming or adverse inflammatory reaction to the compound (FIG. 10). A separate set of trained mice were challenged with LPS (5 μg per mouse), and systemic cytokines were analyzed after 1 hour. A 2.3-fold increase in IL-6 and a 1.97-fold increase in TNF-α levels were observed following LPS challenge suggesting enhanced in-vivo training effects. See FIG. 6B.

Apart from increased systemic cytokines, recruitment of monocyte-derived small peritoneal macrophages (SPMs) is another hallmark of enhanced training. A non-significant increase in the percentage of SPMs was observed two hours post-LPS challenge. See FIG. 6C. Central effects of trained immunity occur at the bone-marrow progenitor favoring myelopoiesis.³⁹ To test if A1155463 induced training effects in bone-marrow cells, cells were isolated from the bone-marrow seven days following the training schedule described in FIG. 6A. Bone-marrow cells from PBS or A1155463 trained cells were challenged with 5 ng/mL of LPS and the supernatant was analyzed 24 hours later. An approximately 5-fold increase was observed in bone-marrow derived IL-6 levels from A1155463 trained mice compared to untrained control. See FIG. 6D. Taken together, these results indicate enhanced in-vivo training effects of A1155463.

To demonstrate the clinical translatability of the A1155463-derived training effects, a B16.F10 tumor model was used. In this model, mice were trained with either vehicle control or A1155463 (15 mg/kg) daily for 6 days. Then, 24 hours post the last dosing, mice were challenged subcutaneously with B16.F10 tumors. See FIG. 6E A significant decrease in tumor growth was observed in A1155463 trained mice compared to the untrained (vehicle) control group. See FIG. 6F. Encouraged by these results, a further study was performed to determine if A1155463 training can be combined with conventional checkpoint therapy to improve anti-tumor effects further.³⁹ The same schedule was as before, but adding two doses of checkpoint inhibitors, an anti-PDL1 and anti-CTLA-4 combination, on days 14 and 17 after the first training regimen. See FIG. 6G. Significantly reduced tumor growth was observed in A1155463 trained mice that were supplemented with checkpoint therapy. See FIG. 6H. Taken together, these results demonstrate that A1155463 mediated training can lead to enhanced antitumor effects. Further studies into the cellular players of this mechanism can reveal novel drug delivery approaches in developing anti-cancer therapeutics.

### Example 7

### Sustained Release Nanoparticles

Trained immunity improves the innate immune response to pathogenic challenge and tumor burden. However, current methods only provide transient approaches to induce training due to the short lifespan of the trained cells. While methods to direct training at the bone-marrow level ensure long-term effects, this approach suffers from the potential disadvantage of inducing long-term adverse inflammatory responses. Precise control of timing and dosage of innate immune cell activation may provide a method to improve training duration while managing unwanted adverse reactions due to prolonged activation.

Methods of controlling the kinetics of trained immunity through a more generalizable approach using the sustained release of trained immunity-inducing molecules from biodegradable nanoparticles were investigated. Poly (lactic-co-glycolic acid) (PLGA) is a widely used FDA-approved polymer that exhibits slow degradation via hydrolysis of ester bonds with broad applications. PLGA nanoparticles can be easily formulated to deliver the encapsulated cargo to their intended target cells. Moreover, the release kinetics can be controlled by modulating the properties of PLGA.

The sustained release of β-glucan from PLGA nanoparticle formulation was investigated in order to determine if prolong trained immunity effects by controlling release kinetics (FIG. 14). PLGA nanoparticles were synthesized that exhibited varied rates of release of β-glucan over 30 d. Enhanced proinflammatory cytokine production was observed in nanoparticle-trained bone-marrow-derived macrophages (BMDMs) challenged with lipopolysaccharide (LPS) in a 7 d in vitro assay. To better capture slow release and its effect on inducing trained immunity, the synthesized nanoparticles were tested in an in vivo model. While the standard dose of 1 mg of free β-glucan conferred trained immunity for more than a week, nanoparticle-trained mice demonstrated peak systemic effects extending over a month of training. This prolonged response enabled the nanoparticle-trained mice to resist tumor growth better than conventionally trained animals when challenged with B16.F10 melanoma three weeks after training. Varying PLGA molecular weight was used to fine-tune the duration of training for various applications by controlling release kinetics. The sustained release from biodegradable polymer nanoparticles for temporal control of trained immunity was achieved over a predictable window of a week to a month.

### Example 8

### Examination of Nanoparticle Encapsulation and Release Efficiency

To test the hypothesis that sustained release can prolong trained immunity, synthesized nanoparticles composed of PLGA encapsulating β-glucan were examined. Nanoparticles were synthesized using a modified version of a previously reported⁴⁰ double emulsion technique, owing to its ease and consistency in creating homogenous nanospheres that degrade in a controlled release fashion. In brief, nanoparticles were synthesized by ultrasonication of an aqueous solution of β-glucan and PLGA dissolved in dichloromethane. The solution was ultrasonicated again after adding 5% PVA and left stirring for 6 h in a 0.5% PVA stabilizing solution. The resulting precipitated nanoparticles were washed and lyophilized for characterization.

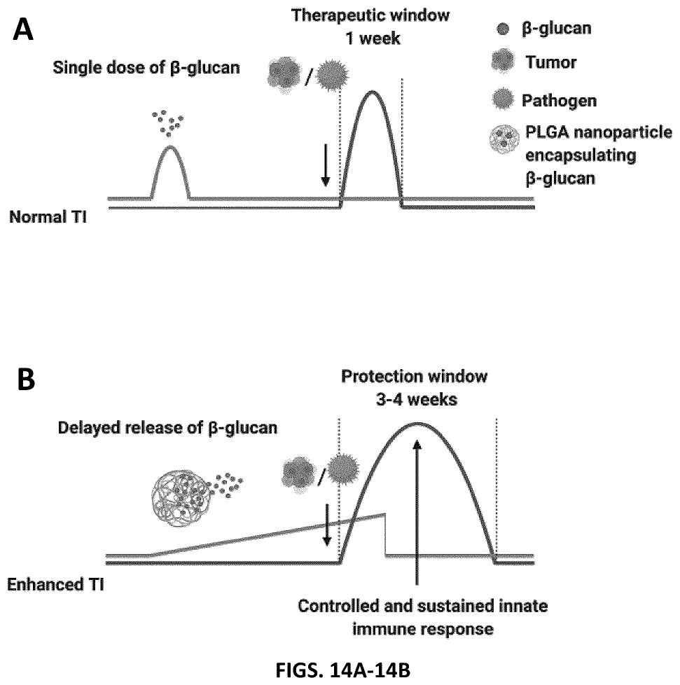

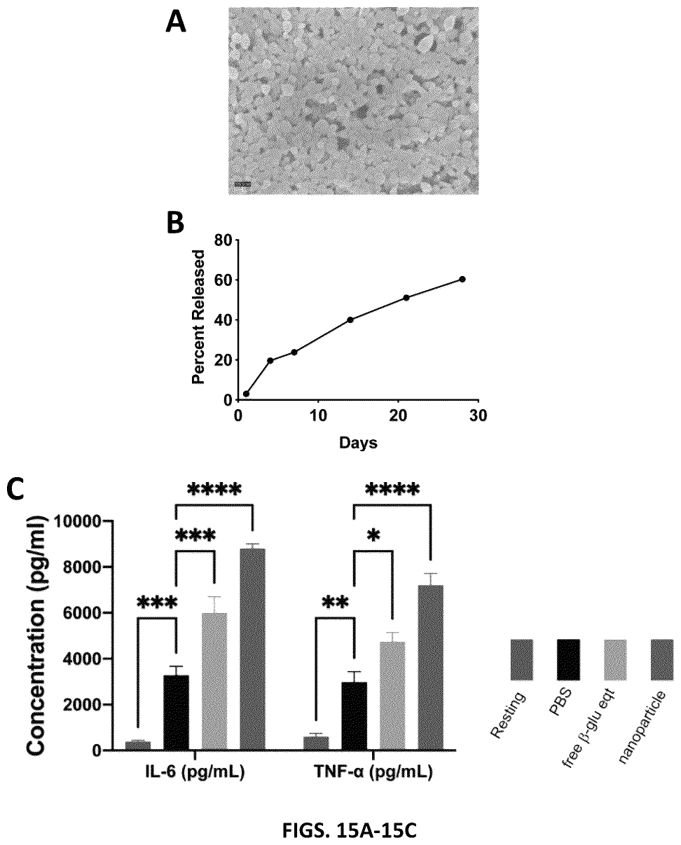

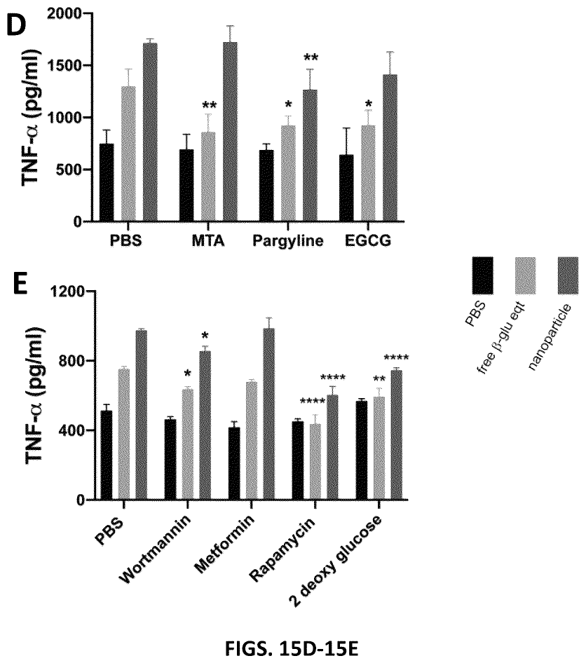

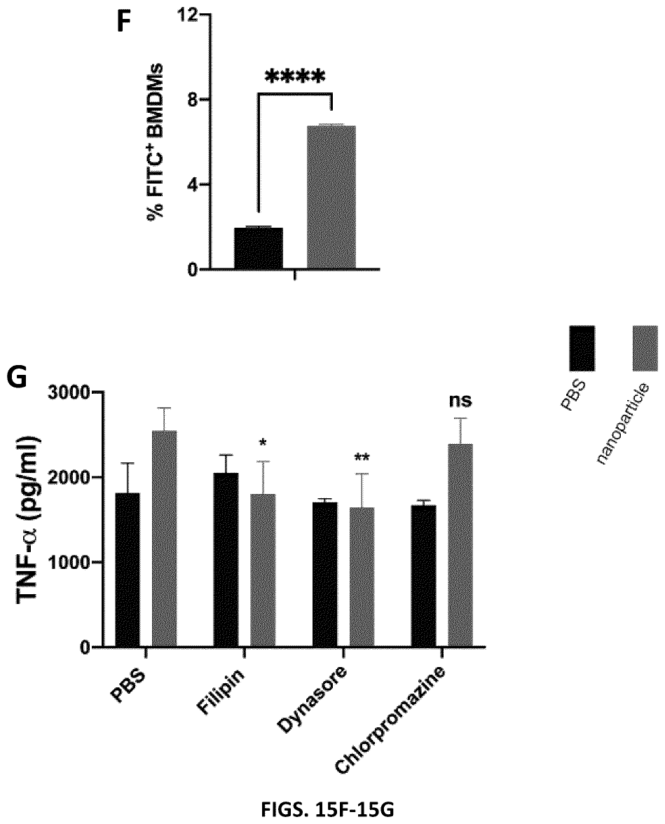

In examining the consistency of nanoparticles, scanning electron microscopy (SEM) images revealed nanosphere morphology of particles that were around 67±20 nm in diameter (FIG. 15A). However, larger radii were detected under DLS measurements suggesting that the particles significantly aggregate in aqueous solutions. The amounts of β-glucan encapsulated within the nanoparticles was quantified using a total carbohydrate assay. Encapsulation efficiency was determined by the percentage of β-glucan as measured by the carbohydrate assay divided by the amount of β-glucan initially loaded in each formulation. Synthesized nanoparticles had a high encapsulation efficiency of 73%. The in vitro release profile was evaluated at pH 7.4, which showed a sustained release profile of β-glucan over the 4 week period of testing (FIG. 15B). The synthesized nanoparticles were confirmed to be devoid of endotoxin using a HEK-mTLR4 reporter assay. Concluding that the particles were loaded with β-glucan and devoid of any other immunostimulatory materials, their ability to train innate cells was then investigated.

Synthesized nanoparticles encapsulating β-glucan were examined for their effects on trained immunity using a standard BMDM training model. In this model, macrophages are trained with added material (e.g., β-glucan) and later challenged with a conventional secondary stimulant (e.g., LPS). If training occurs, the macrophages increase transcription of proinflammatory cytokines like IL-6 and TNF-α. Following a standard 7 d training assay, murine BMDMs at a density of 100 000 cells per well were incubated for 24 h with a set of “training material.” Each group of cells was trained for 24 h, rested for 4 d, and later challenged with 10 ng mL-1 of LPS in a total volume of 200 μL. The release of β-glucan affected training by using nanoparticles was compared using either β-glucan (100 μg mL-1) or administering the free form of an equivalent amount of β-glucan (3.7 μg mL-1). An untrained (or PBS trained) group was included as a negative control. Some nanoparticles were found to stick to the well plate even after washing with PBS after 24 h. Significantly higher IL-6 and TNF-α levels were observed confirming that nanoparticles enhanced trained immunity in vitro (FIG. 15C). In fact, encapsulating the β-glucan in nanoparticles resulted in an approximately 1.5-fold increase in both IL-6 and TNF-α produced by BMDMs in response to the same stimulus compared to the unencapsulated equivalent. In addition, nanoparticles with no β-glucan did not induce training. Together, these results suggest that encapsulation of the training material, β-glucan, could strongly enhance the training effect in preliminary in vitro assays. Additionally, the remaining nanoparticles after the 24 h wash period, might also contribute to slow release of β-glucan and enhanced training.

### Example 9

### Examination of Mechanism of Action

While the initial in vitro data was promising, one important consideration was if encapsulation affected the mechanism of action of trained immunity mediated by β-glucan. In previous experiments, others established that β-glucan induces epigenetic and metabolic changes via histone methylation and acetylation to control transcription of pro-inflammatory cytokines. The nanoparticles were examined in order to determine if they activated similar pathways in trained immunity using our in vitro assay. To do this, BMDMs were pretreated with small-molecule epigenetic and metabolic inhibitors for 30 min before exposing them to each training material in the standard 7 d BMDM training assay. All the epigenetic inhibitors reduced trained immunity induced by free β-glucan, as reported previously—validating the assays. Histone demethylase inhibitor-pargyline hydrochloride significantly reduced training by nanoparticles, whereas histone methyltransferase inhibitor (methylthioadenosine or MTA) did not affect training. Histone acetyltransferase inhibitor (Epigallocatechin-3-gallate or EGCG) reduced nanoparticle-induced training in vitro; however, the difference was not significant (p=0.073) (FIG. 15D). The results confirm that the nanoparticles induce training through an epigenetic mechanism. However, the results also suggest that nanoparticle-based training operates through a slightly different overall temporal process, perhaps owing to the timing and delivery of the β-glucan.

Apart from epigenetics, metabolic changes also contribute to 0-glucan-induced training. Dectin-1 activation by β-glucan results in phosphorylation of Akt, thereby activating the mammalian target of rapamycin (mTOR). Akt inhibitor-wortmannin reduced trained immunity mediated by free β-glucan but not by nanoparticles. Inhibiting mTOR by pretreating cells with rapamycin significantly reduced training effects in both the free β-glucan equivalent and nanoparticle groups. Trained innate cells switch from oxidative phosphorylation to glycolysis to meet the energy demands to induce epigenetic changes for higher effector function, such as secretion of proinflammatory cytokines. This process also generates metabolites that further modulate their epigenetic profile. To test the metabolic effects of training with nanoparticles, glucose was depleted by pre-treating cells with 2-deoxy D-glucose and observed reduced training effects by all training materials tested, confirming that glycolysis is a key to trained immunity effects by nanoparticles (FIG. 15E). Taken together, these results demonstrate that nanoparticles mediate trained immunity through much the same pathways targeted by free β-glucan encompassing both metabolic and epigenetic reprogramming of BMDMs. However, they show distinctive differences in how the particles may alter the pathways, perhaps in the kinetics of the overall dose of β-glucan, which may explain the resulting improvements in training observed in the preliminary in vitro results.

While β-glucan stimulates Dectin-1 and complement receptors, previous reports have shown that trained immunity mediated by β-glucan is not fully dependent on this receptor-ligand interaction. One potential explanation for these subtle mechanistic differences observed with the nanoparticle system would be the kinetics and delivery of β-glucan to the cells. Improved uptake of nanoparticles by BMDMs could be associated with better training in vitro compared to an equivalent amount of free β-glucan. To test this hypothesis, β-glucan was labeled with fluorescein isothiocyanate (FITC) and the monitored uptake by BMDMs at the end of 24 h, mimicking the in vitro training assay using flow cytometry. After 24 h of training, higher levels of FITC+ cells were observed in the nanoparticle trained groups than in the free equivalent group (FIG. 15F). A 7 d in vitro assay was performed by pretreating BMDMs with known inhibitors targeting caveolae and clathrin-mediated endocytosis pathways to test if endocytosis is involved. A significant reduction in nanoparticle-mediated training was observed with inhibitors of both caveolae-specific endocytosis (filipin) and both caveolae and clathrin-dependent endocytosis (dynasore) (FIG. 15G). Clathrin inhibitor (chlorpromazine) did not cause any change in nanoparticle-mediated training. These results strongly support that nanoparticle-induced trained immunity in vitro proceeded through increased uptake by BMDMs revealing an alternate method for inducing trained immunity.

### Example 9

### Nanoparticles in a Prophylactic Model

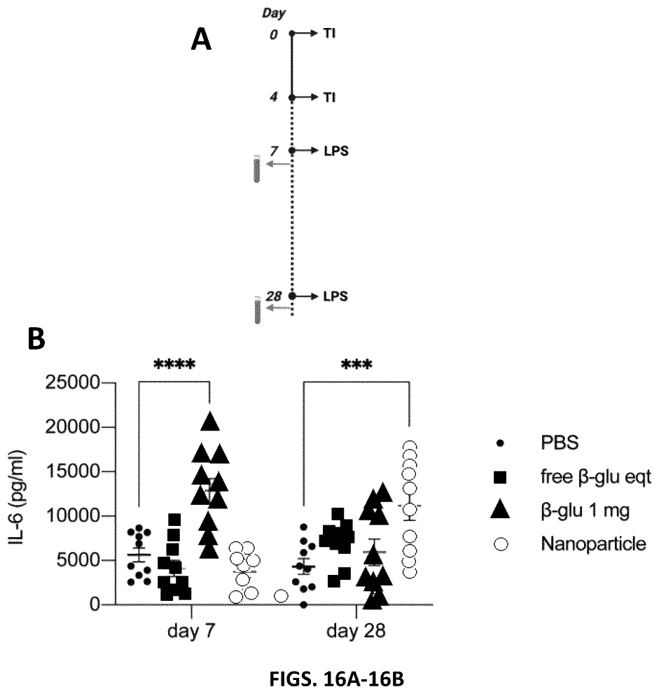

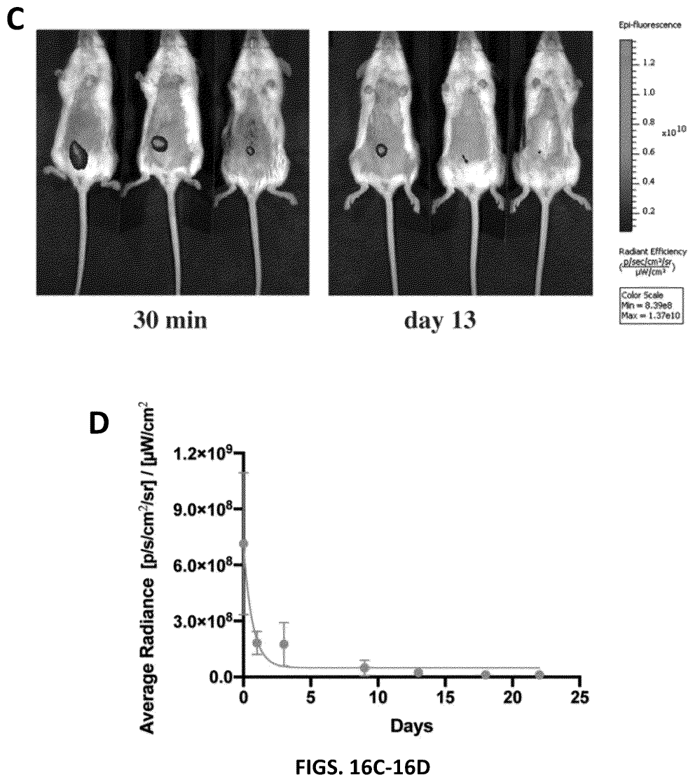

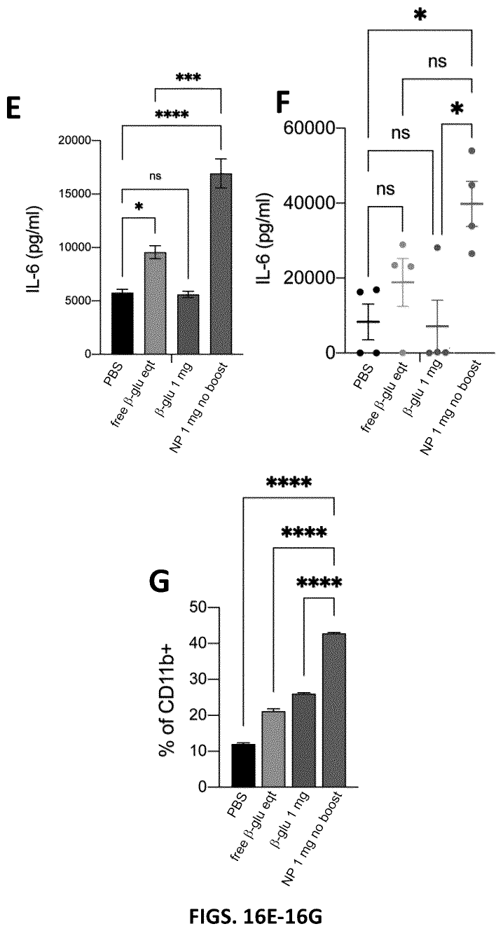

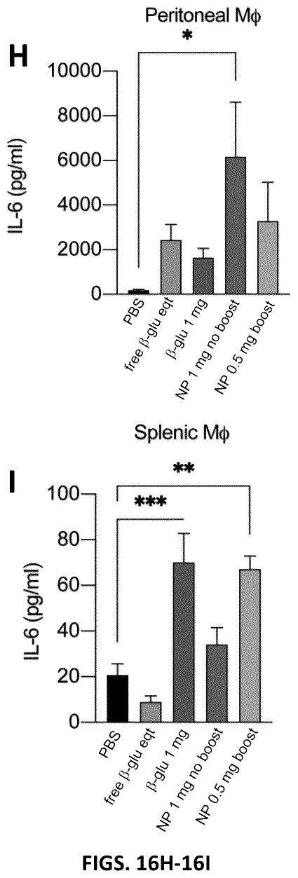

To accurately test how controlled release might affect trained immunity in a prophylactic model, we tested the particles using an in vivo model (FIG. 16A). Mice were trained twice with either nanoparticles or an equivalent amount of free β-glucan. To account for the potential effects of age and injection, an “untrained” (PBS) group was included. For comparison, a conventional standard training regimen 1 mg of free β-glucan was also included. To determine the increase in innate response from training, mice were then challenged intraperitoneally with a secondary stimulus-LPS, at defined time points after the training regimen. In a standard training assay, LPS challenge results in systemic pro-inflammatory cytokines (IL-6 and TNF-α). Higher cytokine response indicates better trained mice. Since the synthesized nanoparticles exhibit slow release of the encapsulated β-glucan over 30 d, the training response in the nanoparticle-trained group was expected to coincide with its release profile, peaking after 30 d of training. To confirm this, systemic cytokines following an LPS challenge at two different time points-7 and 28 d after the first training material was administered. Mice previously trained with standard 1 mg free β-glucan elicited peak systemic cytokines after 7 d of training. At 7 d, for the β-glucan (1 mg) group, systemic cytokines following an LPS challenge were highest, matching previous work. The group trained with the free equivalent amount of β-glucan (35 μg per mouse) did not show any systemic responses. This result could be attributed to the very low dosage only 3.5% of a responsive amount. Also, mice trained with nanoparticles at 7 d somewhat surprisingly showed no systemic response (FIG. 16B). However, when challenged 28 d after the first training, nanoparticle-trained mice showed higher peak systemic responses than mice trained with 1 mg of β-glucan or the nanoparticle-free equivalent dosage. Of note, the level was nearly as high as that of the standard training at day 7 with only 3.5% as much β-glucan added. This result provided strong support for the expectation that the training response in the nanoparticle-trained group was expected to coincide with its release profile, peaking after 30 d of training. Additionally, no increase in cytokines in any category was observed when mice were challenged with LPS 35 d after training, indicating the transient nature of training. To rule out innate immune priming effects, serum cytokines were analyzed before the LPS challenge on day 28. No significant differences were observed between untrained and nanoparticle-trained mice, indicating that the system was not actively responding to previous stimulation. Taken together, these results strongly support that the controlled release from nanoparticles prolonged training, but only peak around 28 d. They did not extend training beyond a predictable time window-preventing potential uncontrolled inflammation.

### Example 10

### Nanoparticles Distribution and Metabolism

To assess the biodistribution of the synthesized nanoparticles, mice were injected intraperitoneally with NIR-labeled PLGA nanoparticles with the same formulation of encapsulated β-glucan (Table Si, Supporting Information). The injected nanoparticles localized to the peritoneal cavity 30 min after injection (FIG. 16C). To confirm that nanoparticles exhibited sustained release, the fluorescence intensity emitted from the nanoparticles was monitored. Fluorescence intensity measurement was reduced to zero by three weeks post-training and followed first-order kinetics. (FIGS. 16C and 16D). After confirming the absence of residual nanoparticles, the mice were challenged with LPS and observed higher systemic cytokines from the nanoparticle-trained mice. These results support the observation that it is the sustained release of β-glucan from the nanoparticles which increases the effect of training. Peak training effects induced in the nanoparticle-trained group was confirmed to occur after the nanoparticles appeared fully dispersed.

After confirming that the nanoparticles localized at the peritoneal cavity, peritoneal macrophages were examined for their contributory effect on the response of nanoparticle-induced training when peak systemic responses were induced at day 28. Peritoneal macrophages were isolated from mice that received training material four weeks earlier. The isolated trained peritoneal macrophages were challenged ex vivo with LPS, and proinflammatory cytokines were quantified. 77% higher IL-6 levels were observed from peritoneal macrophages isolated from nanoparticle-trained mice than those isolated from unencapsulated free equivalent P3-glucan trained mice (FIG. 16E). An adoptive transfer experiment was performed to determine if peritoneal macrophages could act as a sole mediator of training. After 28 d of training in mice administered standard nanoparticles, the free-equivalents of β-glucan, or 1 mg of β-glucan, peritoneal macrophages were adoptively transferred to naïve mice. The recipient mice were challenged with LPS 1 d later, and systemic cytokines were quantified. Analogous to the ex vivo challenge experiment, peritoneal macrophages isolated from nanoparticle-trained mice were observed to confer naïve mice with improved training effects against LPS challenge compared to all other groups tested (FIG. 16F). Mice that received peritoneal macrophages from nanoparticle-trained mice produced a twofold increase in IL-6 compared to the group with an unencapsulated amount of free β-glucan. This result provided evidence that peritoneal macrophages are one of the primary cellular mediators of training for nanoparticle-induced training and that sustained-release platforms can target macrophages to improve training effects.

The nanoparticle-trained mice were examined for localized training effects, particularly at the administration site, i.e., the peritoneal cavity, at earlier times (one week after training). In previous work, inflammation in the peritoneal cavity was observed to induce the recruitment of monocyte-derived small peritoneal macrophages (SPMs). Mice were trained with the same regiment of desired training materials, challenged with LPS after 7 d and their peritoneal cavity cells were harvested. Mice trained with nanoparticles had much higher populations of SPMs (40%) than the free β-glucan groups (FIG. 16G), confirming that the particles induced localized effects. However, training may also modulate immune cell populations at the spleen. Analysis of splenic cell populations between nanoparticles and other free formulations revealed a slight but nonsignificant increase in the percent of macrophages in mice trained with nanoparticles. No changes were observed in other cell types like neutrophils which were upregulated in mice trained with 1 mg free β-glucan. These results indicated that the nanoparticles might exert some localized effects at the injection site.

To explore if the dosing regimen would alter the activity and cellular makeup at the site of action in nanoparticle-induced training at earlier time points (day 7), mice were trained with either two doses of 0.5 mg spaced 4 d apart or a single dose of 1 mg of nanoparticles. In this case, both groups would ultimately release an equivalent amount of β-glucan. Yet, the distributions would differ, resulting, theoretically, in different cellular compositions in the injection site and altered training responses. After a standard training injection of either 0.5 mg of nanoparticles at intervals of 4 d or 1 mg of nanoparticles, cells were collected from the peritoneal cavity, spleen, and bone marrow. Cells were challenged with LPS ex vivo, and cytokines released into the supernatant were analyzed. A single dose of 1 mg nanoparticles resulted in better training in peritoneal macrophages. (FIG. 16H) However, two doses of 0.5 mg of nanoparticles resulted in better-trained immunity effects in splenic macrophages (FIG. 16I). No significant differences were observed in bone marrow-derived macrophages (BMDMs) harvested from nanoparticle trained mice, confirming that the site of action was restricted to the peritoneal cavity and spleen at earlier time points. These results indicate that the dosing regimen of nanoparticles could provide more spatial control over the site of training at earlier time points. Taken together, these results indicate that the training with nanoparticles occurs mainly within the local tissue but can be altered via dosing and distribution.

### Example 11

### Nanoparticle in Cancer Models

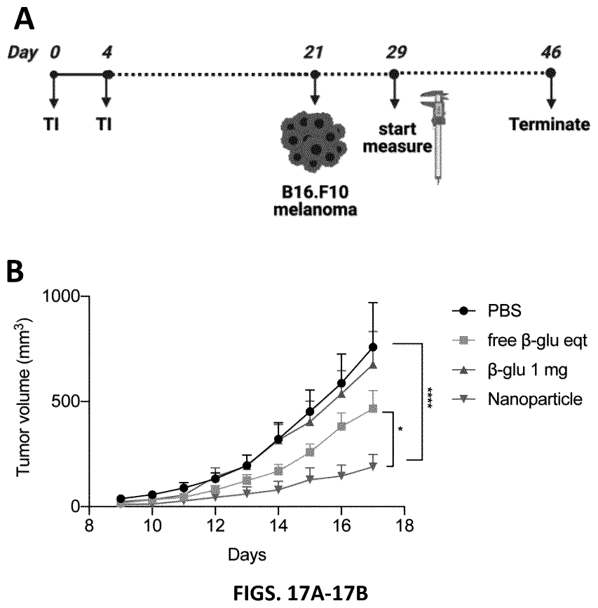

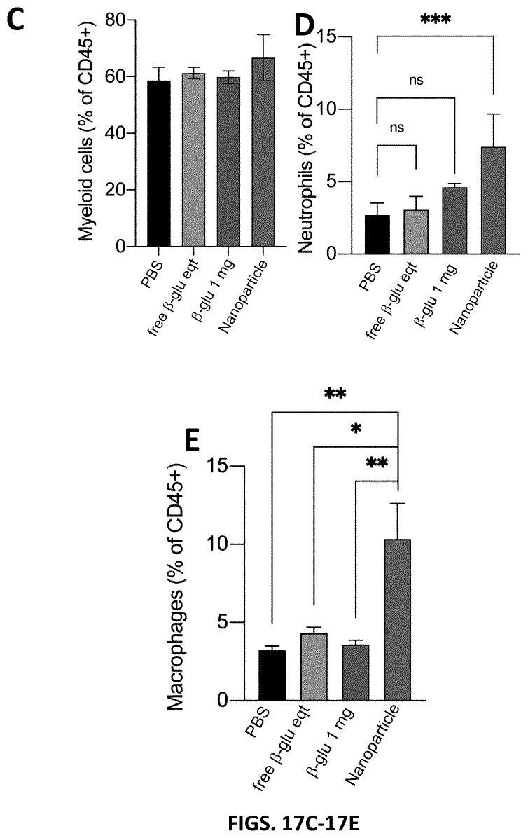

Trained immunity has been proposed and demonstrated to be useful in both infectious diseases and immune therapy. In initial experiments, the temporal persistence of trained immunity was examined for its effects on cancer immuno-therapy, owing to the potential to generate a long-lived response. Generating durable responses against tumor growth is an oft-cited goal to sustain therapeutic effects. Nanoparticle-trained mice were examined for their ability to provide greater resistance to a tumor challenge owing to sustained release. Mice were injected intra-peritoneally, either with nanoparticles containing β-glucan, an equivalent amount of unencapsulated free β-glucan, or a standard training dose of β-glucan at 1 mg. In this experiment, injections were given twice, 4 d apart, indicated as day 0 and day 4. Mice were challenged with B16.F10 tumors three weeks (day 21) after the training. Tumors take about a week to form a visible mass (day 28) which had previously been determined to be the time by which nanoparticles elicited complete release of encapsulated β-glucan (FIG. 17A). Measuring tumor volume, the nanoparticle-trained mice were observed to significantly resist tumor growth compared to the free β-glucan trained groups (FIG. 17B). An analysis of the tumor-infiltrating innate immune cells at the end of the experiment revealed increased neutrophils and macrophages in the tumor microenvironment, confirming that the antitumor effects in nanoparticle-trained mice were due to changes in the tumor microenvironment (FIGS. 17C-17E). Additionally, spleen weights corresponded with tumor size as well splenomegaly was observed for both the PBS and 1 mg free β-glucan groups. Beyond this model, the anti-tumor effects of the nanoparticles were tested in an EG7.OVA tumor model. Similar effects were observed with nanoparticle-trained mice exhibiting the highest tumor resistance. These results suggest that sustained-release training platforms could potentially be combined with existing anticancer therapies, including checkpoint blockade, to prolong protection as they provide an alternate method to influence the tumor microenvironment.

### Example 12

### Nanoparticle Release Kinetics

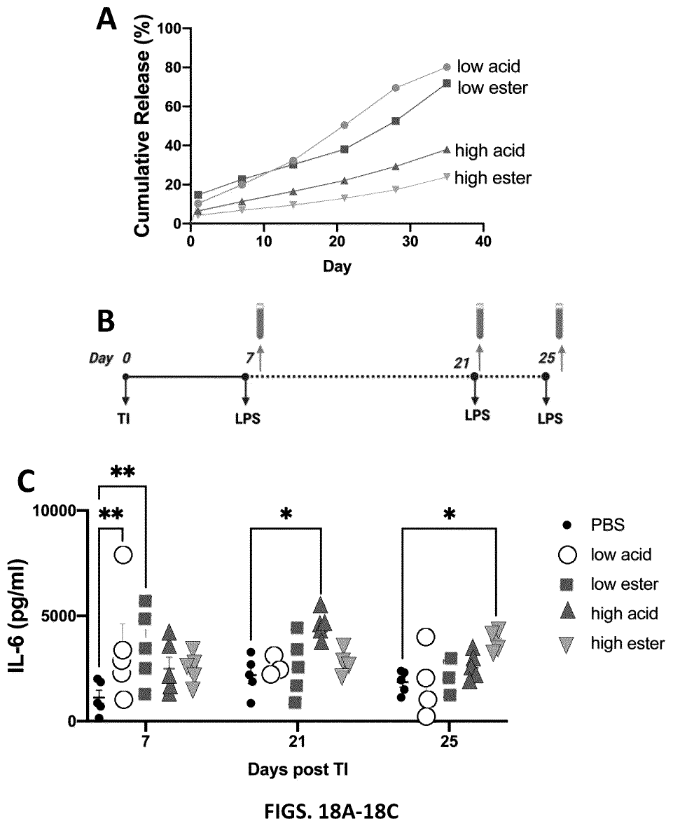

One of the well-established properties of PLGA is its control over the release kinetics of encapsulated cargo. Increasing the molecular weight of PLGA decreases diffusion of the encapsulated cargo, thereby prolonging the duration of release and vice versa. This property might be uniquely suited to improve temporal control of trained immunity. Additionally, PLGA end groups can also affect release kinetics-carboxylic acid end groups degrade faster than those with ester end groups. To test this hypothesis, β-glucan was encapsulated using two different molecular weight PLGA polymers of molecular weights ranging from 7 to 17 kDa and 35-55 kDa. PLGA with two different end groups were also examined-acid terminated and ester terminated. The formulations are hereby categorized as low acid/ester and high acid/ester, respectively. SEM characterization revealed a spherical morphology for all the PLGA nanoparticles. Encapsulation efficiency was in the range of 41-72% for all the synthesized nanoparticles. In vitro release data confirmed that nanoparticles composed of smaller molecular weight PLGA degraded faster than higher molecular weight PLGA nanoparticles (FIG. 18A).

To study the kinetics of training, different molecular weight nanoparticles were tested in an in vivo model with a time-course analysis of serum cytokines following an LPS challenge (FIG. 18B). Mice were trained as previously described with the various molecular weight nanoparticles and challenged with LPS at increasing time points-7, 21, 25, or 30 d after the first training. Peak systemic IL-6 responses were observed at day 7 for both the acid and ester terminated low molecular weight nanoparticles. By day 21, the high molecular weight acid terminated group demonstrated significantly higher systemic IL-6 levels after LPS challenge. However, the high molecular weight ester terminated group peaked at day 25 (FIG. 18C) suggesting that controlled release could be achieved by modulating both molecular weight as well as polymer end groups used. Continued analysis through day 30 confirmed the absence of sustained inflammatory responses. These results further supported the hypothesis that fine-tuning the properties of sustained-release platforms can be used for precise temporal control over the duration of trained immunity. These results suggest that with different formulations, it is possible to partially control the timing of training and lengthen or shorten the window over which higher innate responses are induced via training. Controlled training is an important step to preventing potential over-inflammatory or chronic-inflammatory responses which might cause adverse reactions.

### Example 13

### Discussion

Trained immunity can improve immune responses to pathogens, cancer, and vaccination.^{21,22} Harnessing trained immunity in prophylaxis and treatment can help protect the most vulnerable populations who normally generate muted immune responses.^{24,25} However, few molecules are known to induce training, and the existing repertoire of compounds carry significant limitations and risks.²⁶⁻²⁸

Expanding the number of known inducers of trained immunity, each with unique properties, can improve the ability to control this immunologically ancient process for therapeutic applications.^{29,30} Herein is reported the first high-throughput screen to identify training molecules, thus rapidly expanding the number of noninflammatory inducers of training available. Furthermore, many of these small molecules are ubiquitous pharmaceuticals available in a variety of formulations. The ease of manufacture and delivery of these drugs supports the potential for trained immunity to be used as a clinical strategy for both disease prevention and treatment.

Seven non-immunogenic small molecule inducers of trained immunity were identified that exhibited improved cytokine responses over untrained controls, both in vitro and in vivo. This library of training compounds, spanning diverse classes of drugs ranging from glucocorticoids to adrenergic compounds and others, provided mechanistic insight toward both known and unknown training pathways. The involvement of canonical trained immunity pathways generated by 2 compounds, flunisolide and nerol, was demonstrated using glycolytic and epigenetic inhibitors of training. To improve understanding of these small molecule training pathways, ATAC sequencing can be used to identify differentially accessible genes and transcription factors compared to untrained controls.

The presently disclosed work expands the knowledge of the field of trained immunity. A series of a non-immunogenic trained immunity inducing molecules have been identified. Bone-marrow derived macrophages (BMDMs) trained with A1155463 at nanomolar concentrations secreted higher pro-inflammatory cytokines following an LPS challenge compared to untrained cells. Furthermore, the training mechanism appeared to depend on glycolysis as inhibition with 2-deoxy glucose removed training effects observed in-vitro. A1155463 also improved training effects in an in-vivo model in mice and improved tumor resistance. The anti-tumor effects were further enhanced when combined with checkpoint blockade, demonstrating the potential to use trained immunity to enhance current therapeutics.

### REFERENCES

All references listed herein including but not limited to all patents, patent applications and publications thereof, scientific journal articles, and database entries are incorporated herein by reference in their entireties to the extent that they supplement, explain, provide a background for, or teach methodology, techniques, and/or compositions employed herein.
1. Netea, M. G. et al. Defining trained immunity and its role in health and disease. Nat. Rev. Immunol. 20, 375-388 (2020).
2. Arts, R. J. W. et al. Glutaminolysis and Fumarate Accumulation Integrate Immunometabolic and Epigenetic Programs in Trained Immunity. Cell Metab. 24, 807-819 (2016).
3. Netea, M. G. & van Crevel, R. BCG-induced protection: Effects on innate immune memory. Semin. Immunol. 26, 512-517 (2014).
4. Aaby, P. et al. Randomized Trial of BCG Vaccination at Birth to Low-Birth-Weight Children: Beneficial Nonspecific Effects in the Neonatal Period J. Infect. Dis. 204, 245-252 (2011).
5. Bekkering, S. et al. In Vitro Experimental Model of Trained Innate Immunity in Human Primary Monocytes. Clin. Vaccine Immunol. CVI 23, 926-933 (2016).
6. Kleinnijenhuis, J. et al. Bacille Calmette-Guérin induces NOD2-dependent nonspecific protection from reinfection via epigenetic reprogramming of monocytes. Proc. Natl. Acad. Sci. 109, 17537-17542 (2012).
7. Cheng, S.-C. et al. mTOR- and HIF-1-mediated aerobic glycolysis as metabolic basis for trained immunity. Science 345, 1250684-1250684 (2014).
8. van der Heijden, C. D. C. C. et al. Aldosterone induces trained immunity: the role of fatty acid synthesis. Cardiovasc. Res. 116, 317-328 (2020).
9. van der Heijden, C. D. C. C. et al. Catecholamines Induce Trained Immunity in Monocytes In Vitro and In Vivo. Circ. Res. 127, 269-283 (2020).
10. Edgar, L. et al. Hyperglycemia Induces Trained Immunity in Macrophages and Their Precursors and Promotes Atherosclerosis. Circulation 144, 961-982 (2021).
11. Groh, L. A. et al. oxLDL-Induced Trained Immunity Is Dependent on Mitochondrial Metabolic Reprogramming. Immunometabolism 3, e210025 (2021).
12. King, O. N. F. et al. Quantitative High-Throughput Screening Identifies 8-Hydroxyquinolines as Cell-Active Histone Demethylase Inhibitors. PLOS ONE 5, e15535 (2010).
13. Li, J. et al. A high-throughput screening campaign against PFKFB3 identified potential inhibitors with novel scaffolds. Acta Pharmacol. Sin. 44, 680-692 (2023).
14. Sartori, L. et al. Thieno[3,2-b]pyrrole-5-carboxamides as New Reversible Inhibitors of Histone Lysine Demethylase KDM1A/LSD1. Part 1: High Throughput Screening and Preliminary Exploration. J. Med. Chem. 60, 1673-1692 (2017).
15. Zhou, Y. et al. Development of novel human lactate dehydrogenase A inhibitors: High-throughput screening, synthesis, and biological evaluations. Eur. J. Med. Chem. 177, 105-115 (2019).
16. Fanucchi, S., Dominguez-Andrés, J., Joosten, L. A. B., Netea, M. G. & Mhlanga, M. M. The Intersection of Epigenetics and Metabolism in Trained Immunity. Immunity 54, 32-43 (2021).
17. Eglen, R. M., Gilchrist, A. & Reisine, T. The Use of Immortalized Cell Lines in GPCR Screening: The Good, Bad and Ugly. Comb. Chem. High Throughput Screen. 11, 560-565 (2008).
18. Saz-Leal, P. et al. Targeting SHIP-1 in Myeloid Cells Enhances Trained Immunity and Boosts Response to Infection. Cell Rep. 25, 1118-1126 (2018).
19. Kim, J. Y. et al. Discovery of New States of Immunomodulation for Vaccine Adjuvants via High Throughput Screening: Expanding Innate Responses to PRRs. ACS Cent. Sci. (2023) doi:10.1021/acscentsci.2c01351.
20. Johnston, S. T., Shah, E. T., Chopin, L. K., Sean McElwain, D. L. & Simpson, M. J. Estimating cell diffusivity and cell proliferation rate by interpreting IncuCyte ZOOM™ assay data using the Fisher-Kolmogorov model. BMC Syst. Biol. 9, 38 (2015).
21. Ajit, J. et al. Temporal Control of Trained Immunity via Encapsulated Release of β-Glucan Improves Therapeutic Applications. Adv. Healthc. Mater. 11, 2200819 (2022).
22. Ciarlo, E. et al. Trained Immunity Confers Broad-Spectrum Protection Against Bacterial Infections. J. Infect. Dis. 222, 1869-1881 (2020).
23. Oxidized Low-Density Lipoprotein Induces Long-Term Proinflammatory Cytokine Production and Foam Cell Formation via Epigenetic Reprogramming of Monocytes | Arteriosclerosis, Thrombosis, and Vascular Biology. https://www.ahajournals.org/doi/10.1161/ATVBAHA.114.303887.
24. Maródi, L. Innate cellular immune responses in newborns. Clin. Immunol. 118, 137-144 (2006).
25. Montecino-Rodriguez, E., Berent-Maoz, B. & Dorshkind, K. Causes, consequences, and reversal of immune system aging. J. Clin. Invest. 123, 958-965 (2013).
26. Chan, G. C.-F., Chan, W. K. & Sze, D. M.-Y. The effects of β-glucan on human immune and cancer cells. J. Hematol. Oncol. J Hematol Oncol 2, 25 (2009).
27. Hesseling, A. C. et al. The risk of disseminated Bacille Calmette-Guerin (BCG) disease in HIV-infected children. Vaccine 25, 14-18 (2007).
28. Norouzi, S., Aghamohammadi, A., Mamishi, S., Rosenzweig, S. D. & Rezaei, N. Bacillus Calmette-Gudrin (BCG) complications associated with primary immunodeficiency diseases. J. Infect. 64, 543-554 (2012).
29. Netea, M. G. & Meer, J. W. M. van der. Trained Immunity: An Ancient Way of Remembering. Cell Host Microbe 21, 297-300 (2017).
30. Mulder, W. J. M., Ochando, J., Joosten, L. A. B., Fayad, Z. A. & Netea, M. G. Therapeutic targeting of trained immunity. Nat. Rev. Drug Discov. 18, 553-566 (2019).
31. Benjamini, Y; Krieger, A. M.; Yekutieli, D. Adaptive linear step-up procedures that control the false discovery rate. Biometrika 93(3), 491-507 (2006).
32. Bruey, J.-M. et al. Bcl-2 and Bcl-XL Regulate Proinflammatory Caspase-1 Activation by Interaction with NALP1. Cell 129, 45-56 (2007).
33. Badrichani, A. Z. et al. Bcl-2 and Bcl-XL serve an anti-inflammatory function in endothelial cells through inhibition of NF-κB. J Clin Invest 103, 543-553 (1999).
34. Giménez-Cassina, A. & Danial, N. N. Regulation of mitochondrial nutrient and energy metabolism by BCL-2 family proteins. Trends in Endocrinology & Metabolism 26, 165-175 (2015).
35. Yi, C. H. et al. Metabolic Regulation of Protein N-Alpha-Acetylation by BclxL Promotes Cell Survival. Cell 146, 607-620 (2011).
36. Vander Heiden, M. G. et al. Bcl-xl Promotes the Open Configuration of the Voltage-dependent Anion Channel and Metabolite Passage through the Outer Mitochondrial Membrane. Journal of Biological Chemistry 276, 19414-19419 (2001).
37. Chen, Y. et al. Bcl-xL regulates mitochondrial energetics by stabilizing the inner membrane potential. Journal of Cell Biology 195, 263-276 (2011).
38. Tao, Z.-F. et al. Discovery of a Potent and Selective BCL-XL Inhibitor with in Vivo Activity. ACS Med Chem Lett 5, 1088-1093 (2014).
39. Mitroulis, I. et al. Modulation of Myelopoiesis Progenitors Is an Integral Component of Trained Immunity. Cell 172, 147-161.e12 (2018).
40. B. A. Bailey, L. J. Ochyl, S. P. Schwendeman, J. J. Moon, Adv. Healthcare Mater. 2017, 6, 1601418.
   It will be understood that various details of the presently disclosed subject matter may be changed without departing from the scope of the presently disclosed subject matter. Furthermore, the foregoing description is for the purpose of illustration only, and not for the purpose of limitation.

1. A method of inducing trained immunity in a subject, the method comprising administering a small molecule selected from the group consisting of a glucocorticoid, A1155463, nerol, hydroquinone, 5-fluoroindole-2-carboxylic acid, myricetin, fenoterol, a pharmaceutically acceptable salt thereof, and any combination thereof.

2. The method of claim 1, wherein administering the small molecule leads to an increase in concentration of at least one cytokine that is at least 1.6-fold greater than that which would have occurred if the subject had not been administered the small molecule.

3. (canceled)

4. The method of claim 2, wherein the at least one cytokine is tumor necrosis factor-alpha (TNF-α) or interleukin-6 (IL-6).

5. (canceled)

6. (canceled)

7. (canceled)

8. (canceled)

9. (canceled)

10. (canceled)

11. The method of claim 1, wherein the small molecule is a glucocorticoid selected from the group consisting of flunisolide, hydrocortisone, hydrocortisone acetate, halcinonide, budesonide, fluocinonide, fluocinolone acetonide, clobetasol propionate, prednisolone, triamcinolone, triamcinolone diacetate, nitrofurazone, and benseraside hydrochloride.

12. (canceled)

13. The method of claim 1, wherein the small molecule does not induce a primary immune response.

14. (canceled)

15. (canceled)

16. (canceled)

17. (canceled)

18. (canceled)

19. The method of claim 1, wherein the small molecule is encapsulated in a nanoparticle comprising a biodegradable polymer selected from the group consisting of polyesters, polyamides, polyurethanes, polyureas, poly(amide-enamine)s, polyanhydrides, polyvinyl alcohols, polyacrylates, proteins, polysaccharides, natural rubber, and polyolefins.

20. (canceled)

21. (canceled)

22. (canceled)

23. The method of claim 1, wherein the small molecule induces a future pro-inflammatory response in the subject that occurs when the cell or subject is exposed to an immunological challenge at a time point at least one week after said administration.

24. (canceled)

25. The method of claim 23, wherein the immunological challenge is selected from an infectious agent, a secondary metastasis, or a vaccine.

26. (canceled)

27. The method of claim 23, wherein the immunological challenge is a secondary metastasis and the subject is a subject having previously been treated for a primary cancer.

28. (canceled)

29. A method of preventing an infection in a subject, wherein the method comprises administering to the subject a small molecule selected from the group consisting of a glucocorticoid, A1155463, nerol, hydroquinone, 5-fluoroindole-2-carboxylic acid, myricetin, fenoterol, a pharmaceutically acceptable salt thereof, and any combination thereof.

30. The method of claim 29, wherein the administering is performed at least one week prior to contact by, or expected risk of contact by, the subject with an infectious agent.

31. (canceled)

32. (canceled)

33. (canceled)

34. (canceled)

35. (canceled)

36. (canceled)

37. (canceled)

38. (canceled)

39. The method of claim 29, wherein the small molecule is a glucocorticoid selected from the group consisting of flunisolide, hydrocortisone, hydrocortisone acetate, halcinonide, budesonide, fluocinonide, fluocinolone acetonide, clobetasol propionate, prednisolone, triamcinolone, triamcinolone diacetate, nitrofurazone, and benseraside hydrochloride.

40. (canceled)

41. The method of claim 29, wherein the small molecule does not induce a primary immune response.

42. The method of claim 29, wherein the small molecule is encapsulated in a nanoparticle comprising a biodegradable polymer selected from the group consisting of polyesters, polyamides, polyurethanes, polyureas, poly(amide-enamine)s, polyanhydrides, polyvinyl alcohols, polyacrylates, proteins, polysaccharides, natural rubber, and polyolefins.

43. (canceled)

44. (canceled)

45. (canceled)

46. The method of claim 29, wherein administration of the small molecule causes an increase in concentration of at least one cytokine that is at least 1.6-fold greater than that which would have occurred if the subject had not been contacted with the small molecule.

47. (canceled)

48. The method of claim 46, wherein the at least one cytokine is tumor necrosis factor-alpha (TNF-α) or interleukin-6 (IL-6).

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. A method of modulating the activity of BCL pathway family member protein in a cell of a subject, comprising administering to the subject a modulating effective amount of a small molecule selected from the group consisting of a glucocorticoid, A1155463, nerol, hydroquinone, 5-fluoroindole-2-carboxylic acid, myricetin, and fenoterol, a pharmaceutically acceptable salt thereof, and any combination thereof.

95. (canceled)

96. The method of claim 94, wherein the glucocorticoid is selected from the group consisting of flunisolide, hydrocortisone, hydrocortisone acetate, halcinonide, budesonide, fluocinonide, fluocinolone acetonide, clobetasol propionate, prednisolone, triamcinolone, triamcinolone diacetate, nitrofurazone, and benseraside hydrochloride.

97. (canceled)

98. (canceled)

99. (canceled)

100. (canceled)

101. (canceled)

102. (canceled)

103. (canceled)

104. (canceled)

105. (canceled)

106. A composition for inducing trained immunity comprising a nanoparticle and a small molecule selected from the group consisting of a glucocorticoid, A1155463, nerol, hydroquinone, 5-fluoroindole-2-carboxylic acid, myricetin, and fenoterol, a pharmaceutically acceptable salt thereof, and any combination thereof, wherein the small molecule is encapsulated in the nanoparticle.

107. The composition of claim 106, wherein the glucocorticoid is selected from the group consisting of flunisolide, hydrocortisone, hydrocortisone acetate, halcinonide, budesonide, fluocinonide, fluocinolone acetonide, clobetasol propionate, prednisolone, triamcinolone, triamcinolone diacetate, nitrofurazone, and benseraside hydrochloride.

108. The composition of claim 106, wherein the nanoparticle comprises a biodegradable polymer selected from the group consisting of polyesters, polyamides, polyurethanes, polyureas, poly(amide-enamine)s, polyanhydrides, polyvinyl alcohols, polyacrylates, proteins, polysaccharides, natural rubber, and polyolefins.

109. (canceled)

110. The method of claim 108, wherein the polyester is poly(lactic-co-glycolic acid).
